(19) United States
(12) Patent Application Publication
(10) Pub. No.: US 2025/0114609 A1
(43) Pub. Date: Apr. 10, 2025

(54) SYSTEMS AND METHODS FOR PROVIDING CARDIAC PULMONARY NERVE STIMULATION

(71) Applicant: Cardionomic, Inc., New Brighton, MN (US)

(72) Inventors: Julio C. Spinelli, Lakewood Ranch, FL (US); Jeremy A. Schaefer, Lake Elmo, MN (US); David C. Olson, Eden Prairie, MN (US); Michael S. Brandner, Andover, MN (US); Imad Libbus, St. Paul, MN (US); Seyed S. Salavatian, Pittsburgh, PA (US)

(21) Appl. No.: 18/906,668

(22) Filed: Oct. 4, 2024

Related U.S. Application Data
(60) Provisional application No. 63/588,123, filed on Oct. 5, 2023.

(51) Int. Cl. A61N 1/36

(52) U.S. Cl. CPC A61N 1/36139; A61N 1/36053; A61N 1/36057; A61N 1/36114; A61N 1/3615; A61N 1/36171; A61N 1/36175; A61N 1/36185

(57) ABSTRACT

A system and method for providing cardiac pulmonary nerve stimulation (CPNS) with therapy titration, a goodness indicator, and therapy weaning. Therapy titration can include using physiological measurements of contractility, lusitropy, and chronotropy to determine therapy efficacy and adjust therapy delivery. For example, the electrode position and stimulation intensity can be adjusted based on the physiological measurements. Therapy titration can include closed-loop therapy delivery, in which therapy is automatically adjusted based on the measured physiological parameters. A goodness indicator can be used to monitor cardiovascular parameters such as contractility, preload, lusitropy, systolic function, and/or chronotropy. The goodness indicator can determine whether the change in these parameters from nerve stimulation is positive, and further indicate optimal stimulation. Therapy weaning can include gradually reducing stimulation while ensuring no significant reduction in therapeutic efficacy ensues.

### INCORPORATION BY REFERENCE TO ANY PRIORITY APPLICATIONS

Any and all applications for which a foreign or domestic priority claim is identified in the Application Data Sheet as filed with the present application are hereby incorporated in their entireties by reference under 37 CFR 1.57. In particular, this application claims priority to the U.S. Provisional Application 63/588,123, filed Oct. 5, 2023, which is incorporated by reference herein in its entirety.

### BACKGROUND

### Field

The present disclosure relates generally to methods and systems for facilitating modulation (e.g., electrical neuromodulation), including methods and systems for facilitating therapeutic and calibration electrical neuromodulation (e.g., stimulation) of one or more nerves in and around the heart (e.g., cardiac pulmonary nerve stimulation).

### Description of the Related Art

Acute heart failure is a cardiac condition in which a problem with the structure or function of the heart impairs its ability to supply sufficient blood flow to meet the body's needs. The condition impairs quality of life and is a leading cause of hospitalizations and mortality in the western world. Treating acute heart failure is typically aimed at removal of precipitating causes, prevention of deterioration in cardiac function, and control of the patient's congestive state.

### SUMMARY

Systems and methods described herein relate to providing modulation (e.g., stimulation) to a neural target, including but not limited to cardiac pulmonary nerve stimulation (CPNS), with (i) therapy titration, (ii) a goodness indicator or other multifactor efficacy indicator, algorithm or protocol for evaluating physiological response to neural stimulation as a way of determining optimal stimulation, and/or (iii) therapy weaning. Therapy titration can include, for example, using physiological measurements of contractility, lusitropy, chronotropy, and/or dromotropy to determine therapy efficacy (or likelihood of therapy efficacy) and adjust therapy delivery (e.g., therapy parameters or locations). For example, the electrode position, the electrode configuration and/or stimulation intensity and/or other parameters can be adjusted (e.g., in real time or substantially in real time) based on the physiological measurements. Therapy titration can include closed-loop therapy delivery, in which therapy is automatically adjusted by execution of program instructions stored in a non-transitory computer-readable storage medium by one or more processors based on the measured physiological parameters. Therapy titration could additionally or alternatively involve non-automated adjustment by a clinician or other user. A goodness indicator or other therapy optimization structure, algorithm or protocol can be used to monitor cardiovascular parameters such as contractility, preload, lusitropy, systolic function, and/or chronotropy. The goodness indicator or other therapy optimization structure, algorithm or protocol can determine whether the change in these parameters from nerve stimulation is positive (or as desired or likely to be efficacious), and further indicate optimal stimulation (e.g., electrode location or configuration and/or stimulation parameters). Therapy weaning can include gradually reducing stimulation while ensuring no significant adverse effects ensue. This can allow for the maintenance of effective CPNS therapy at a lower stimulation intensity or duty cycle. The weaning may occur automatically as part of a closed-loop therapy delivery regime or protocol.

In accordance with several implementations, the goal of CPNS can be to force a restart of the local control circuit of the intracardiac nervous system (ICNS) through stimulation of the cardiac plexus at the level of the right pulmonary artery. The ICNS can include a network of intracardiac ganglia and interconnecting neurons that regulate cardiac activity. Dysfunction of the ICNS can cause heart diseases or irregularities, such as arrythmias. ICNS function can be rehabilitated by forcing it to restart and determining that its regulation is benefitting the patient or subject.

CPNS therapy can trigger the ICNS to signal to the cardiac autonomic nervous system (CANS) that it is receiving excessive sympathetic stimulation. The systems and methods described herein can include selecting a stimulation vector that sets the ICNS to a state that improves hemodynamics, and therefore cerebral blood flow. The systems and methods described herein, combined or in isolation, can prompt the cardiac autonomic nervous system to reassess its current working state and allow the system (e.g., CANS, ICNS) to shift from the current maladaptive state (which can cause acute decompensated heart failure or other conditions) to a virtuous cycle (which can improve the patient condition even after stimulation ends). This may result in improved cardiac output, improved coronary flow, improved renal function, increased diuresis, and/or improved autonomic balance.

Embodiments of the disclosure advantageously provide one or more of the following benefits: (i) reduced side effects and/or reduced adverse events due to reduced stimulation intensity; (ii) reduced battery use and/or increased longevity (e.g., for embodiments involving chronically implantable device; and/or (iii) reduction of habituation and/or maintenance of therapeutic response.

Embodiments of the disclosure may be applicable with respect to any system that delivers stimulation to a neural target, including an acutely implanted neural stimulation system, a chronically implanted neural stimulation system, and/or a non-implantable neural stimulation system (e.g., a transcutaneous neural stimulation system).

Treatments for acute heart failure include the use of inotropic agents, such as dopamine and dobutamine. These agents, however, have both chronotropic and inotropic effects and characteristically increase heart contractility at the expense of significant increases in oxygen consumption secondary to elevations in heart rate. As a result, although these inotropic agents increase myocardial contractility and improve hemodynamics, clinical trials have consistently demonstrated excess mortality caused by cardiac arrhythmias and increase in myocardium oxygen consumption.

As such, there is a need for selectively and locally treating acute heart failure and otherwise achieving hemodynamic control without causing unwanted systemic effects. Accordingly, in some examples, no inotropics are used. In other examples, reduced dosages of inotropics may be used because, for example, synergistic effects are provided through various examples herein. By reducing the dosages, the side effects can also be significantly reduced.

Several examples of the present disclosure relate to methods of tissue modulation, such as neuromodulation, for cardiac and other disorders. For example, some examples provide methods and devices for neuromodulation of one or more nerves in and around a heart of a patient. Several methods of the present disclosure, for example, may be useful in electrical neuromodulation of patients with cardiac disease, such as patients with acute or chronic cardiac disease. Several methods of the present disclosure encompass, for example, neuromodulation of one or more target sites of the autonomic nervous system of the heart. In some examples, sensed non-electrical heart activity properties are used in making adjustments to one or more properties of the electrical neuromodulation delivered to the patient. Non-limiting examples of medical conditions that can be treated according to the present disclosure include cardiovascular medical conditions.

As discussed herein, the configuration of the catheter and electrode systems of the present disclosure may advantageously allow for a portion of the catheter to be positioned within the vasculature of the patient in the main pulmonary artery and/or one or both of the pulmonary arteries (the right pulmonary artery and the left pulmonary artery). Once positioned, the catheter and electrode systems of the present disclosure can provide electrical stimulation energy (e.g., electrical current or electrical pulses) to stimulate the autonomic nerve fibers surrounding the main pulmonary artery and/or one or both of the pulmonary arteries in an effort to provide adjuvant cardiac therapy to the patient.

Several methods of the present disclosure allow for electrical neuromodulation of the heart of the patient, for example including delivering one or more electrical pulses through a catheter positioned in a pulmonary artery of the heart of the patient, sensing from at least a first sensor positioned at a first location within the vasculature of the heart one or more heart activity properties (e.g., a non-electrical heart activity property) in response to the one or more electrical pulses, and adjusting a property of the one or more electrical pulses delivered through the catheter positioned in the pulmonary artery of the heart in response to the one or more heart activity properties. The methods may provide adjuvant cardiac therapy to the patient.

Sensing from at least the first sensor positioned at the first location can include sensing one or more of a pressure property, an acceleration property, an acoustic property, a temperature, and a blood chemistry property from within the vasculature of the heart. Among other locations, the first sensor can be positioned in one of a left pulmonary artery, a right pulmonary artery, a pulmonary artery branch vessel, or a pulmonary trunk of the heart. The one or more electrical pulses can optionally be delivered through the catheter positioned in one of the left pulmonary artery, the right pulmonary artery, or pulmonary trunk of the heart that does not contain the first sensor. The first sensor can also be positioned in a pulmonary trunk of the heart.

Other locations for the first sensor can include in the right ventricle of the heart and in the right atrium of the heart. When positioned in the right atrium of the heart, the first sensor can optionally be positioned on the septal wall of the right atrium of the heart. The first sensor could also be positioned on the septal wall of the right ventricle. The right ventricle and the left ventricle share a septal wall, so a sensor in the right ventricle or on the septal wall of the right ventricle may be preferable for detecting properties indicative of left ventricle contraction. In certain examples, the effect on heart contractility is to increase heart contractility, relaxation, and/or cardiac output. Additional locations for positioning the first sensor include in a superior vena cava of the heart, the inferior vena cava of the heart, and in a coronary sinus of the heart. When positioned in the coronary sinus of the heart, the first sensor can be used to sense at least one of a temperature or a blood oxygen level.

In some examples, the first sensor may be positioned in the left atrium (e.g., by forming an aperture in the septal wall between the right atrium and the left atrium, or by using a patent foramen ovale (PFO) or atrial septal defect (ASD)). A sensor in the left atrium may be useful for detecting properties indicative of the left ventricle. If the left atrium has been accessed, in some examples, the sensor may be positioned in the left ventricle itself, which may provide the most direct measurement of properties associated with the left ventricle. In some examples, the sensor may be positioned downstream of the left ventricle, including the aorta, aortic branch arteries, etc. When the procedure is complete, any aperture that was created or existing may be closed using a closure device such as Amplatzer, Helex, CardioSEAL, or others. Other measurements of left ventricle contractility can include invasive methods, for example, positioning a strain gauge on the myocardium to measure changes in myocardial stretch, positioning an electrode in proximity to a left stellate ganglion to measure single or multi-unit activity, and/or positioning a cuff electrode around sympathetic fibers to measure neural activity, for example compound action potentials.

Some methods can include sensing one or more cardiac properties from a skin surface of the patient, and adjusting the property of the one or more electrical pulses delivered through the catheter positioned in the pulmonary artery of the heart in response to the one or more heart activity properties (e.g., non-electrical properties) from the first sensor positioned at a first location within the vasculature of the heart and/or the one or more cardiac properties from the skin surface of the patient. The one or more cardiac properties sensed from the skin surface of the patient can include, for example, an electrocardiogram property.

Some methods can include sensing from at least a second sensor positioned at a second location within the vasculature of the heart one or more heart activity properties (e.g., non-electrical heart activity properties) in response to the one or more electrical pulses, and adjusting the property of the one or more electrical pulses delivered through the catheter positioned in the pulmonary artery of the heart in response to the one or more heart activity properties from the first sensor and/or the one or more heart activity properties from the second sensor.

Adjusting the property of the one or more electrical pulses can include a variety of responses. For example, adjusting the property of the one or more electrical pulses can include changing which of an electrode or plurality of electrodes on the catheter is used to deliver the one or more electrical pulses. For another example, adjusting the property of the one or more electrical pulses can include moving the catheter to reposition one or more electrodes of the catheter in the pulmonary artery of the heart. For yet another example, adjusting the property of the one or more electrical pulses can include changing at least one of an electrode polarity, a pulsing mode, a pulse width, an amplitude, a frequency, a phase, a voltage, a current, a duration, an inter-pulse interval, a duty cycle, a dwell time, a sequence, a wavelength, and/or a waveform of the one or more electrical pulses.

A hierarchy of electrode configurations can be assigned from which to deliver the one or more electrical pulses. The one or more electrical pulses can be delivered based on the hierarchy of electrode configurations, where the one or more heart activity properties sensed in response to the one or more electrical pulses can be analyzed and an electrode configuration can be selected to use for delivering the one or more electrical pulses through the catheter positioned in the pulmonary artery of a heart of a patient based on the analysis. A hierarchy can be assigned to each property of the one or more electrical pulses delivered through the catheter positioned in the pulmonary artery of the heart, where the one or more electrical pulses are delivered based on the hierarchy of each property. The one or more non-electrical heart activity properties sensed in response to the one or more electrical pulses are analyzed and an electrode configuration can be selected to be used for delivering the one or more electrical pulses through the catheter positioned in the pulmonary artery of a heart of a patient based on the analysis. Analyzing the one or more heart activity properties can include analyzing a predetermined number of the one or more heart activity properties.

The first parameter may be one of the following: a polarity, a pulsing mode, a pulse width, an amplitude, a frequency, a phase, a voltage, a current, a duration, an inter-pulse interval, a duty cycle, a dwell time, a sequence, a wavelength, a waveform, or an electrode combination, and, optionally, the second parameter may be a different one of the following: a polarity, a pulsing mode, a pulse width, an amplitude, a frequency, a phase, a voltage, a current, a duration, an inter-pulse interval, a duty cycle, a dwell time, a sequence, a wavelength, a waveform, or an electrode combination. The second parameter may be one of the following: a polarity, a pulsing mode, a pulse width, an amplitude, a frequency, a phase, a voltage, a current, a duration, an inter-pulse interval, a duty cycle, a dwell time, a sequence, a wavelength, a waveform, or an electrode combination. The first parameter may comprise current and the second parameter may comprise a parameter relating to timing (e.g., one of frequency and duty cycle).

In one example, a method for optimizing neurostimulation therapy can include measuring a cardiovascular parameter at a first point in time, applying stimulation at a vector to a nerve proximal to a pulmonary artery (e.g., one or more nerves of a cardiac plexus), measuring the cardiovascular parameter at a second point in time, calculating a change in the cardiovascular parameter, and determining therapy efficacy of the stimulation at the vector based on the change in the cardiovascular parameter.

The method can include measuring the cardiovascular parameter at a third point in time, applying stimulation at a second vector to the nerve proximal to the pulmonary artery, measuring the cardiovascular parameter at a fourth point in time, calculating a change in the cardiovascular parameter, and determining therapy efficacy of the stimulation at the second vector based on the change in the cardiovascular parameter. The method can include applying the stimulation at the vector with higher therapy efficacy. The cardiovascular parameter can include, for example, contractility, preload, and/or chronotropy. The vector can include at least one of electrode position, electrode configuration, or electrode combination.

In another example, a method for optimizing neurostimulation therapy can include measuring a plurality of cardiovascular parameters at a first point in time, the plurality of cardiovascular parameters comprising at least two of contractility, preload, and/or chronotropy. The cardiovascular parameters may be used to generate (e.g., calculate) a multi-factor efficacy indicator based on a plurality of cardiovascular parameters. The method also includes applying stimulation to a nerve (e.g., one or more nerves of a cardiac plexus) at a vector, measuring the cardiovascular parameters at a second point in time, calculating changes in the plurality of cardiovascular parameters, and determining therapy efficacy based on the changes in the plurality of cardiovascular parameters (e.g., changes in the multi-factor efficacy indicator generated from the plurality of cardiovascular parameters).

The cardiovascular parameters can include, for example, lusitropy and/or systolic blood pressure. Measuring contractility can include measuring at least two dimensions of contractility.

In another example, a method for optimizing neurostimulation therapy can include applying a first stimulation to a nerve proximal to a pulmonary artery (e.g., one or more nerves of a cardiac plexus), measuring a cardiovascular parameter at a first point in time, applying a second stimulation to the nerve in the pulmonary artery with a reduced intensity, measuring the cardiovascular parameter at a second point in time, calculating a change in the cardiovascular parameter, and determining a therapeutic response of the second stimulation based on the change in the cardiovascular parameter. The cardiovascular parameter may be a multi-factor efficacy indicator based on a plurality of cardiovascular parameters.

The method can include, upon detecting the therapeutic response above a threshold, applying a third stimulation to the nerve in the pulmonary artery with a further reduced intensity. The method can include, upon detecting the therapeutic response below a threshold, applying a third stimulation with a less reduced intensity. The method can include, upon detecting the therapeutic response below a threshold, stopping applying stimulation.

In another example, a method of treating heart failure (e.g., acute decompensated heart failure) can include applying stimulation to one or more nerves of a cardiac plexus via one or more electrodes positioned within a right pulmonary artery of a subject. The stimulation can include pulses having a frequency between 2 Hz and 40 Hz, a pulse width of 100 μs to 4 ms and an intensity of 0.1 mA to 20 mA. The stimulation can be applied for a duration of 20 seconds to 120 seconds at intervals of 5 minutes to 20 minutes according to a sequence. Other stimulation and parameters and durations, such as the ranges of parameters and durations disclosed herein, may be used as desired and/or required.

The sequence can be repeated for 2 hours and then stopped for 2 hours according to a cycle. The cycle can be repeated for 48 hours. The intensity of the stimulation over a final weaning cycle can be gradually reduced. Other durations, such as the ranges of durations disclosed herein, may be used as desired and/or required.

In another example, a system for treating heart failure can include a catheter comprising a plurality of electrodes and a stimulator comprising a pulse generator configured to deliver stimulation pulses to one or more of the plurality of electrodes of the catheter. The stimulation pulses can have a frequency of between 2 Hz and 40 Hz, a pulse width of 100 μs to 4 ms, and an intensity of 0.1 mA to 20 mA. Other stimulation parameters, such as the ranges of stimulation parameters disclosed herein, may be used as desired and/or required.

Trains of stimulation pulses can be delivered for 20 seconds to 120 seconds and then stopped for 5 minutes to 20 minutes according to a sequence. The sequence can be repeated for 2 hours and then stopped for 2 hours according to a cycle. The cycle can be repeated for 48 hours. Other durations, such as the ranges of durations disclosed herein, may be used as desired and/or required.

For purposes of summarizing the disclosure, certain aspects, advantages, and novel features have been described herein. It is to be understood that not necessarily all such aspects, advantages, or features may be achieved in accordance with any particular embodiment of the disclosure disclosed herein. Thus, the embodiments disclosed herein may be embodied or carried out in a manner that achieves or optimizes one advantage or group of advantages as taught or suggested herein without necessarily achieving other advantages as may be taught or suggested herein. The systems and methods of the disclosure each have several innovative aspects, no single one of which is solely responsible for the desirable attributes disclosed herein. The methods summarized above and set forth in further detail below may describe certain actions taken by a practitioner; however, it should be understood that they can also include the instruction of those actions by another party. The present disclosure will be more fully understood from the following detailed description of the examples thereof, taken together with the drawings, claims and descriptions above.

### BRIEF DESCRIPTION OF THE DRAWINGS

Non-limiting features of some embodiments are set forth with particularity in the claims that follow. The following drawings are for illustrative purposes only and show non-limiting embodiments. Features from different figures may be combined in several embodiments. It should be understood that the figures are not necessarily drawn to scale. Distances, angles, etc. are merely illustrative and do not necessarily bear an exact relationship to actual dimensions and layout of the devices illustrated.

FIG. 1 schematically illustrates a system that can be used to apply electrical neuromodulation to one or more nerves in and around the heart of a subject.

FIG. 2A schematically illustrates a heart and surrounding areas.

FIGS. 2B-2D are schematic illustrations of a heart and surrounding areas from various perspectives.

FIGS. 2E and 2F are schematic illustrations of a heart and surrounding nerves.

FIGS. 2G and 2H are schematic illustrations of vasculature and an electrode matrix.

FIG. 21 is a schematic illustration of heart vasculature and surrounding nerves.

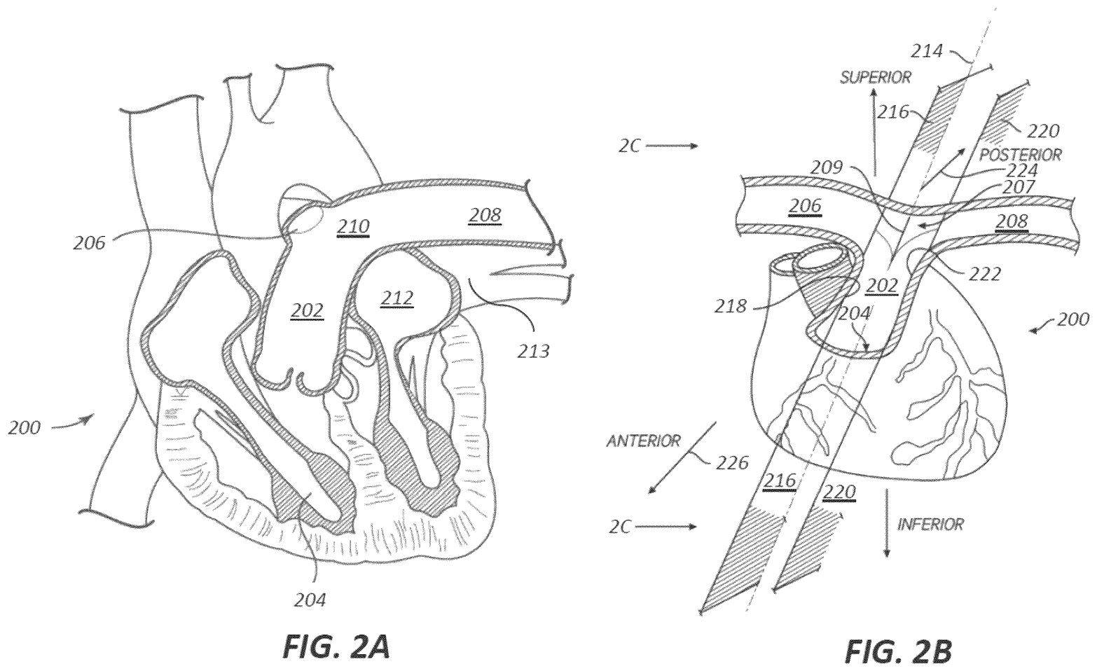

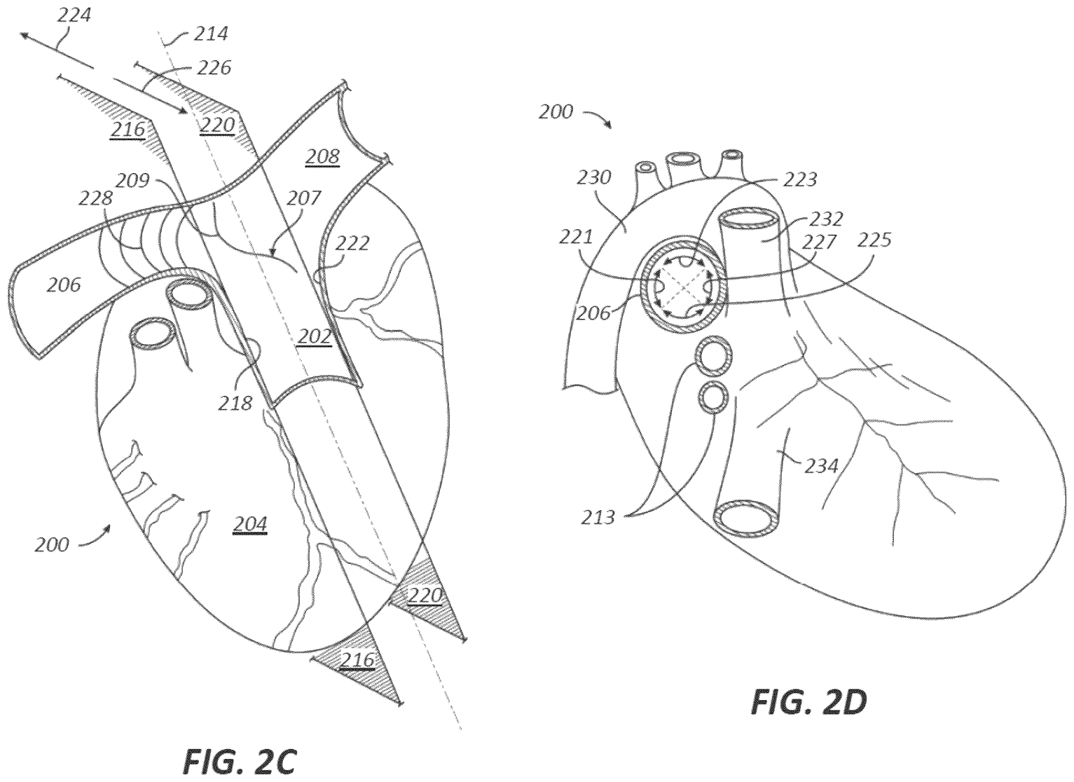

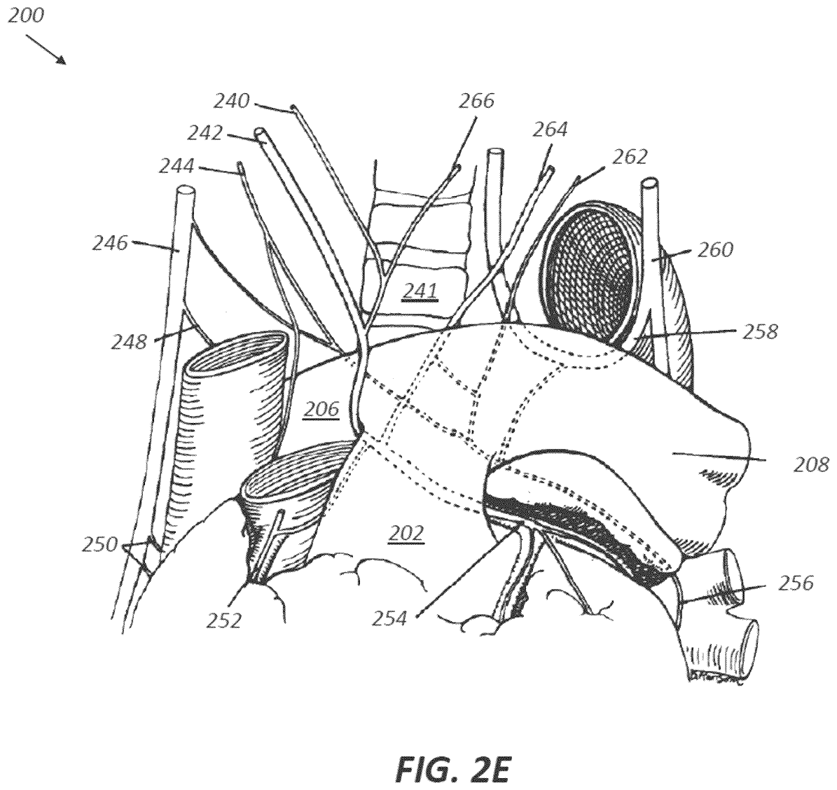

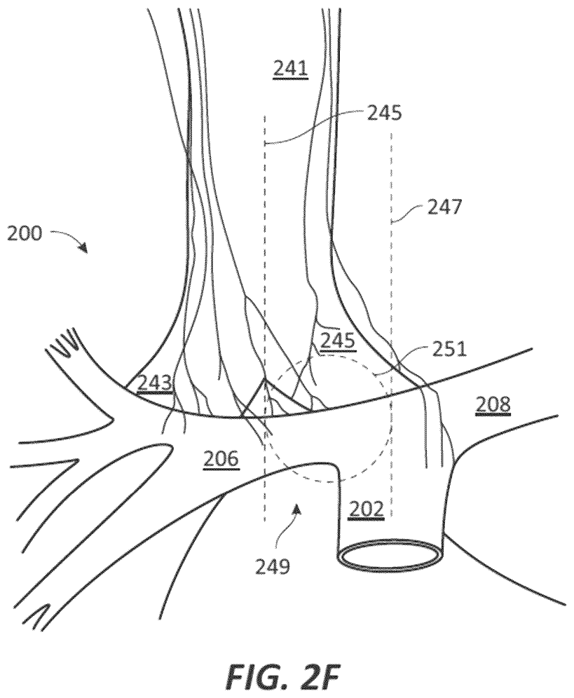

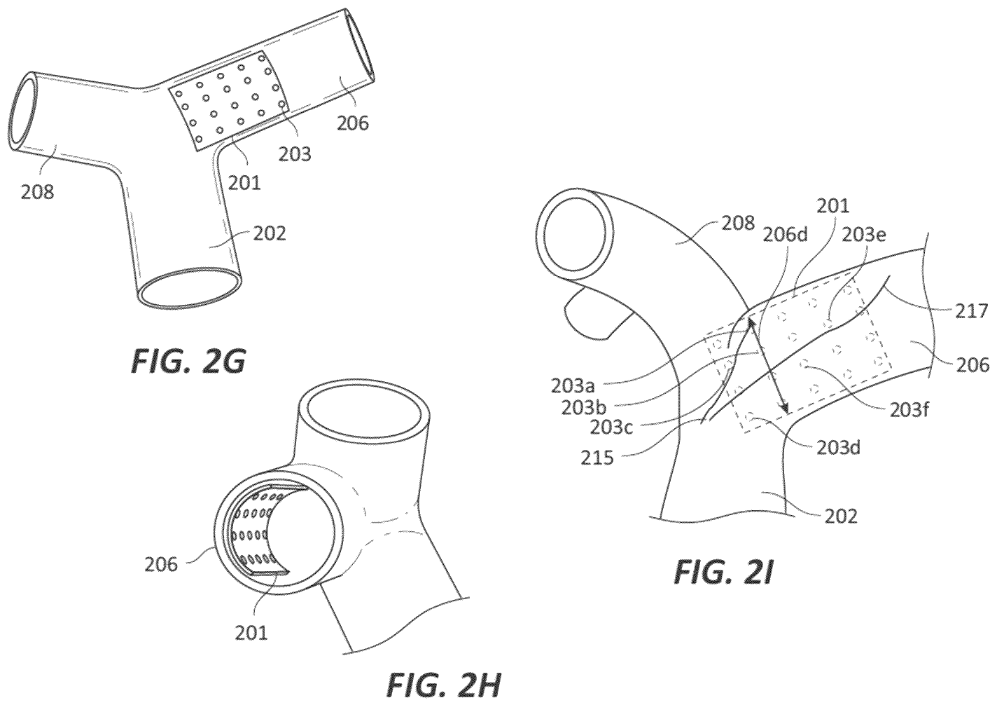

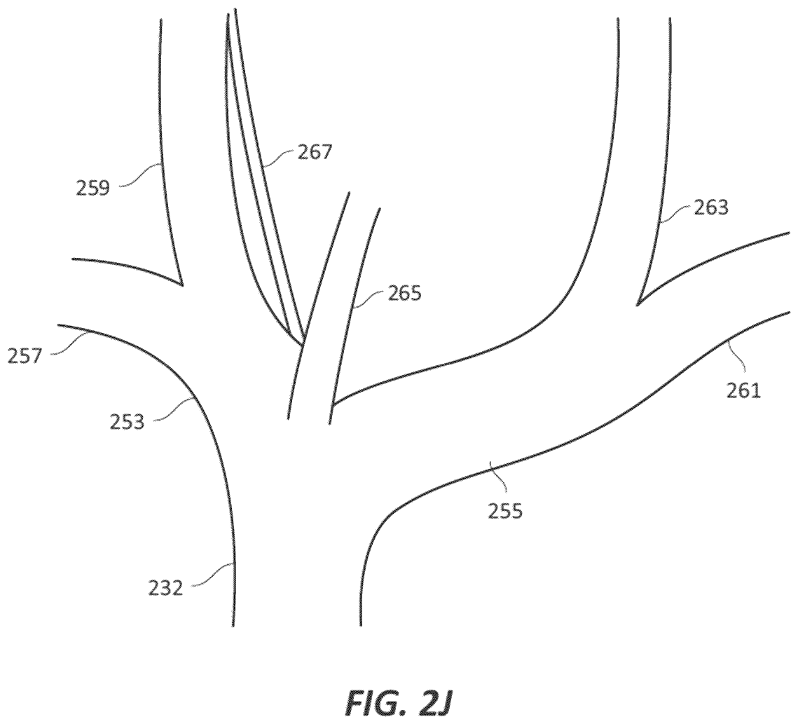

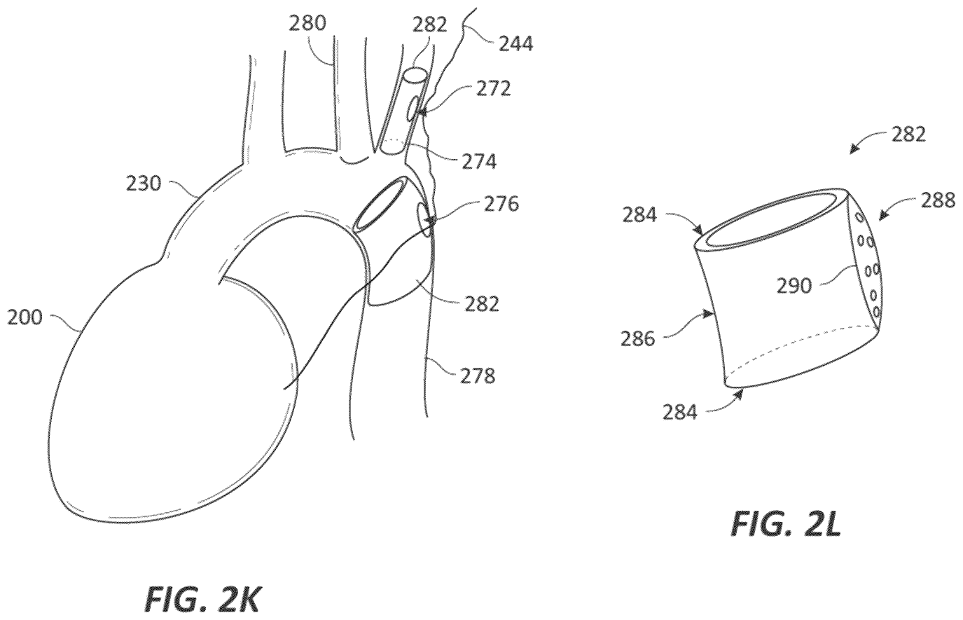

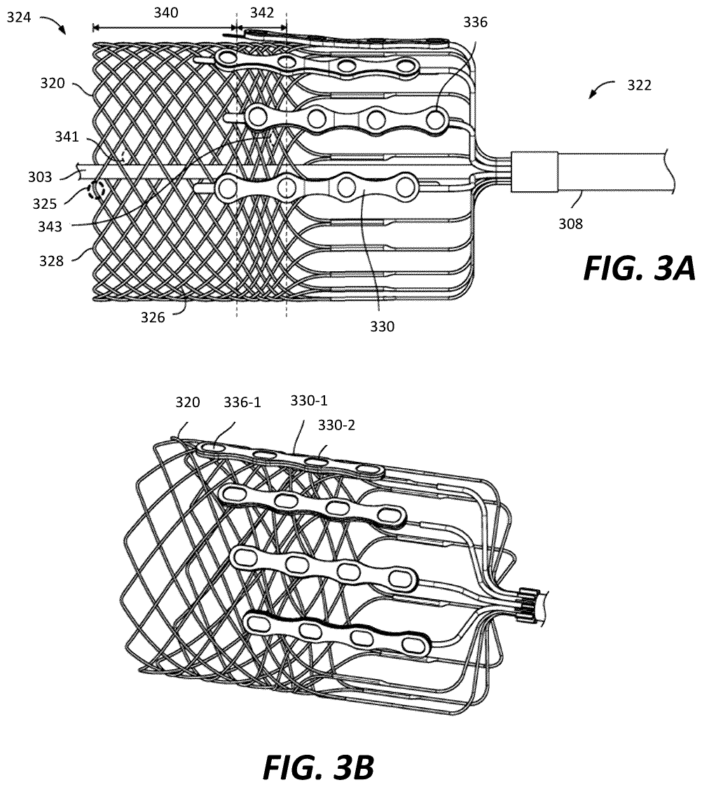

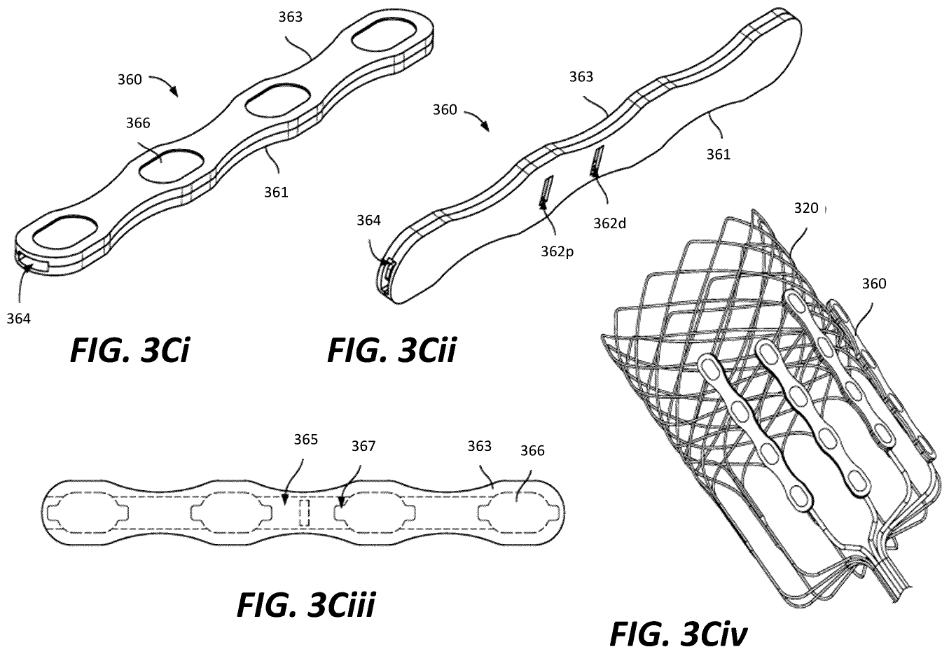

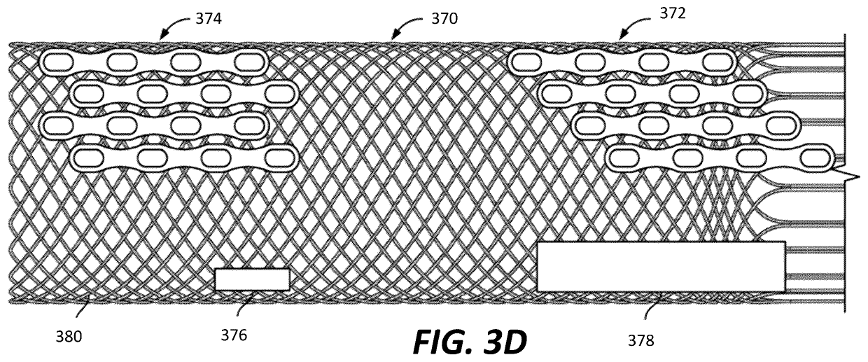

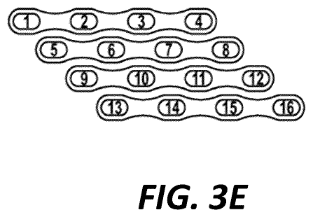

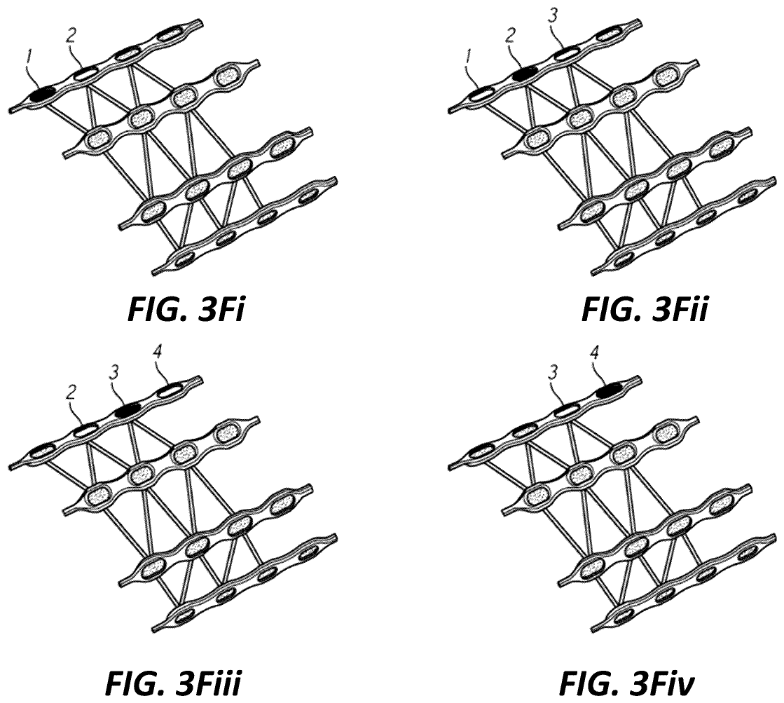

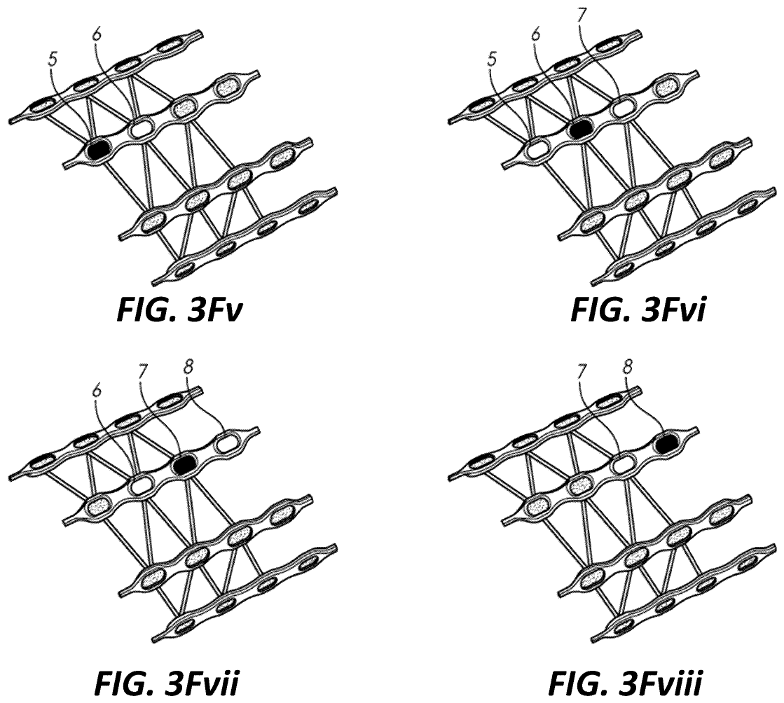

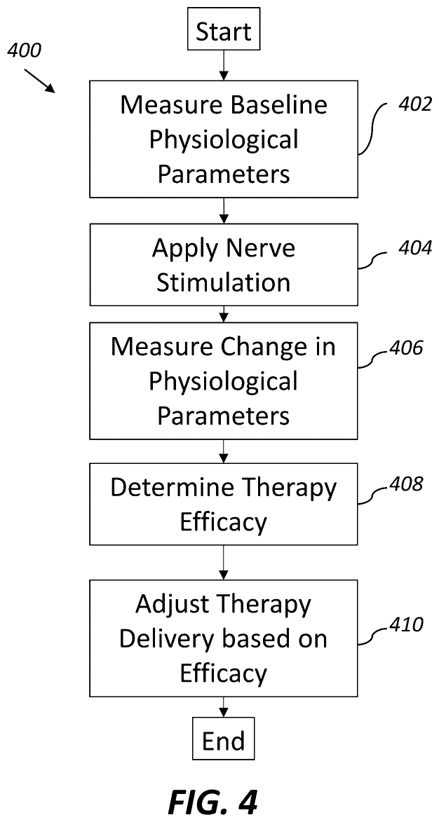

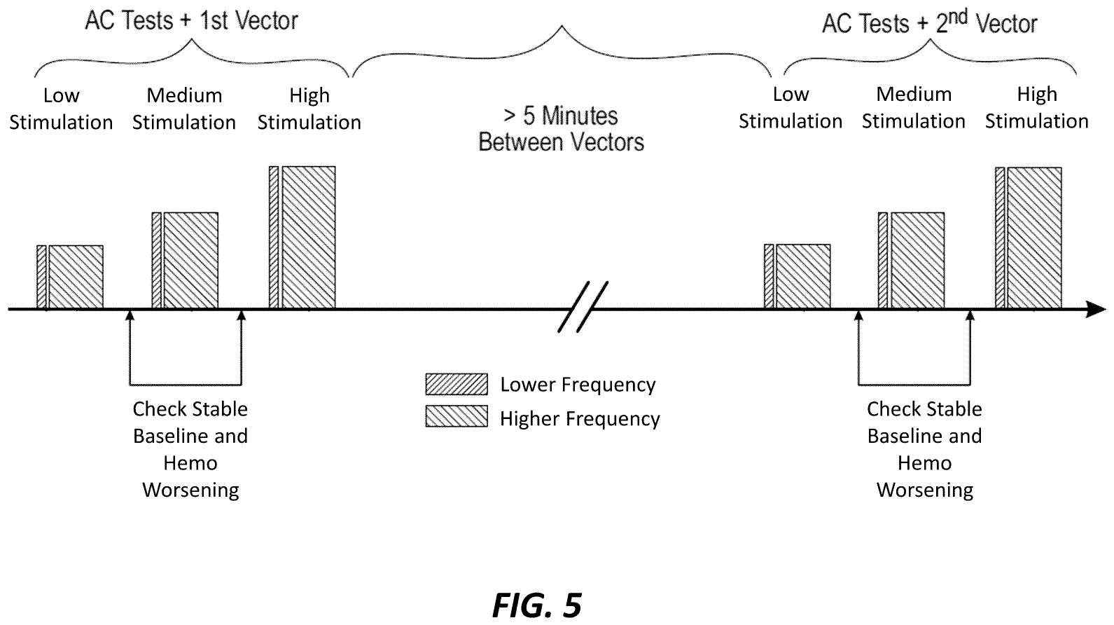

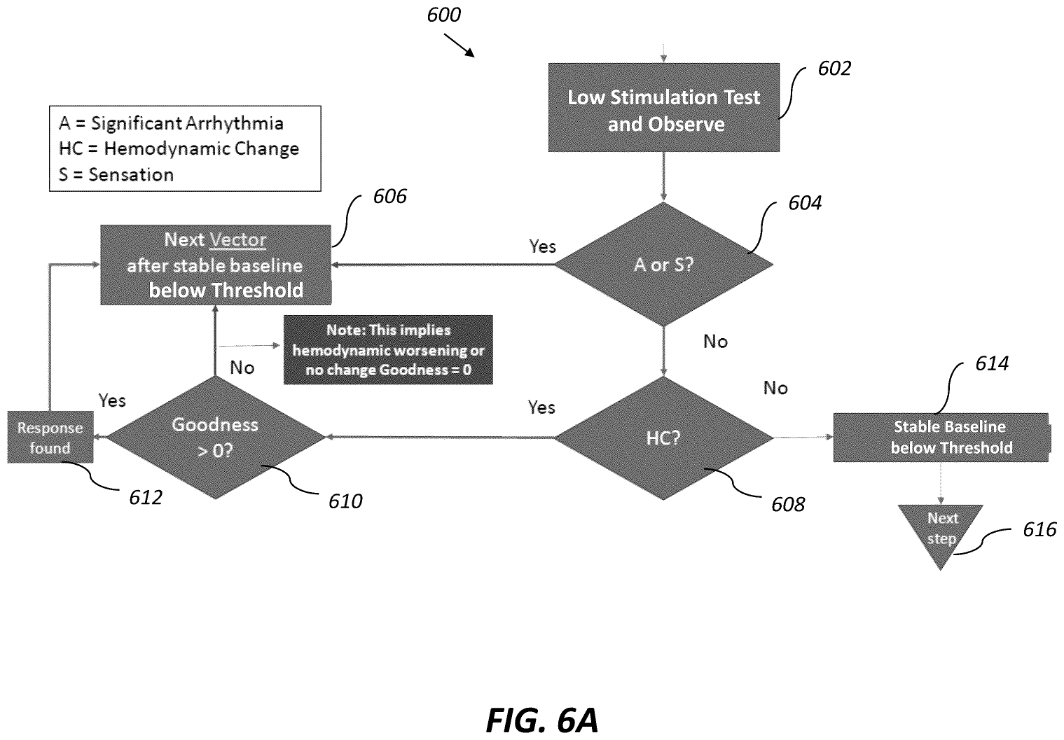

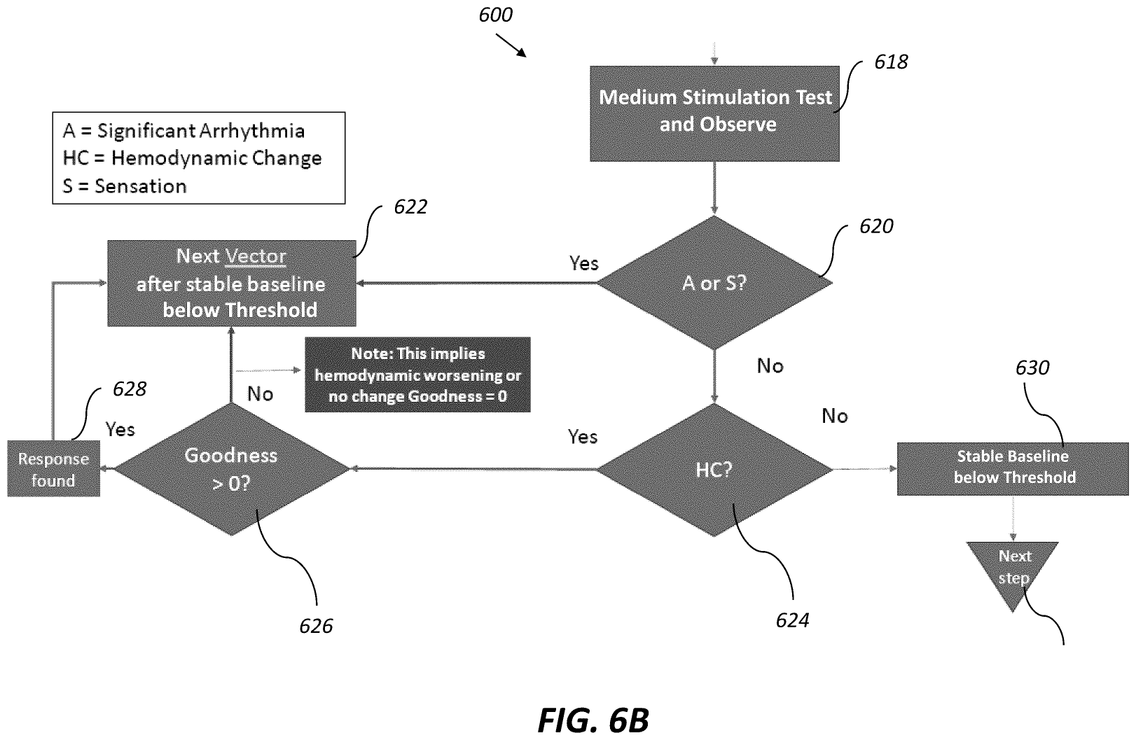

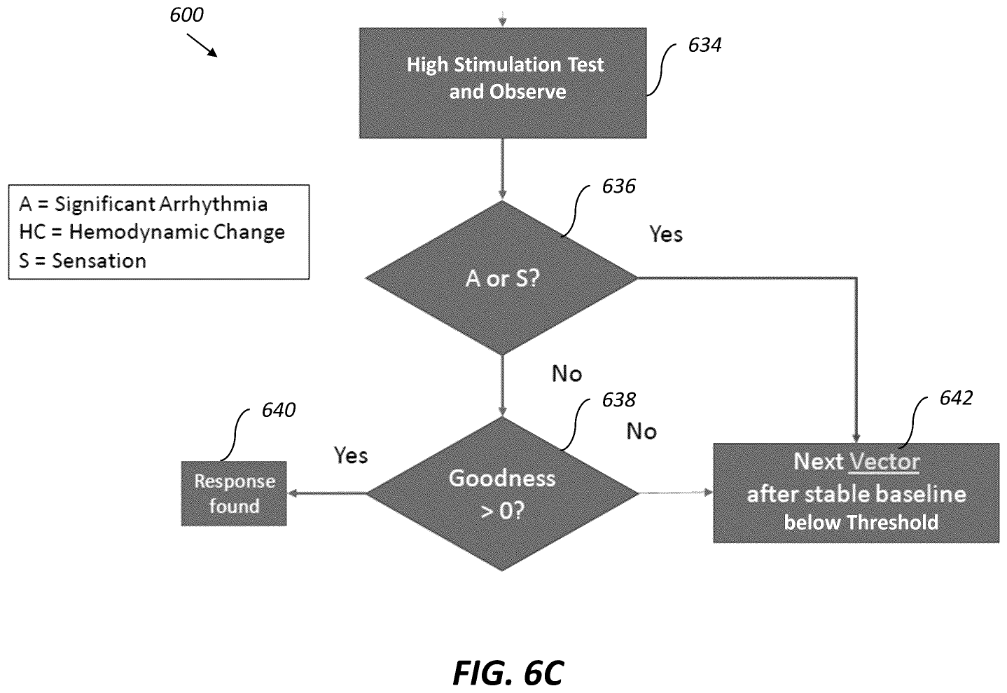

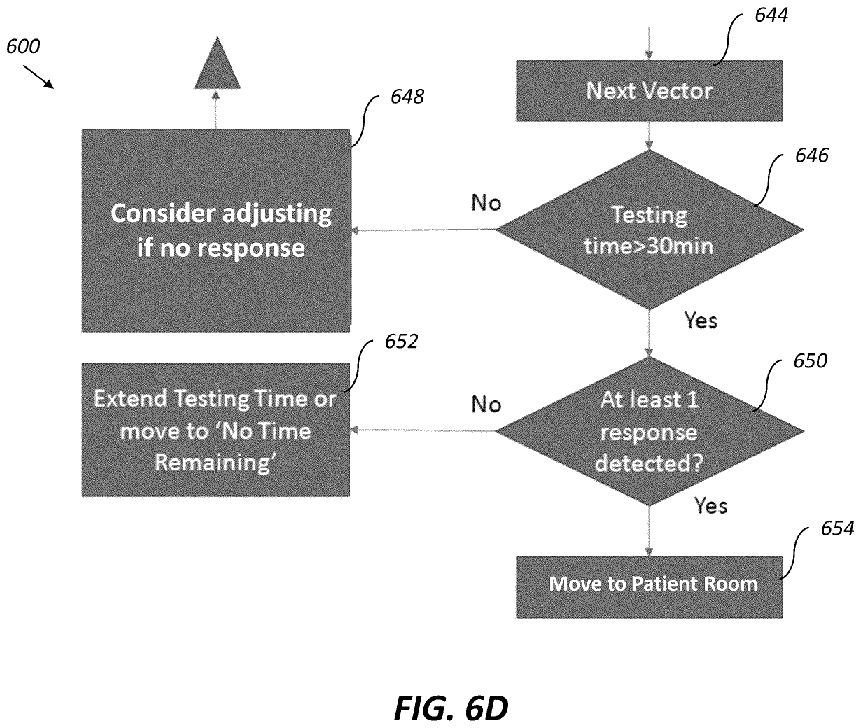

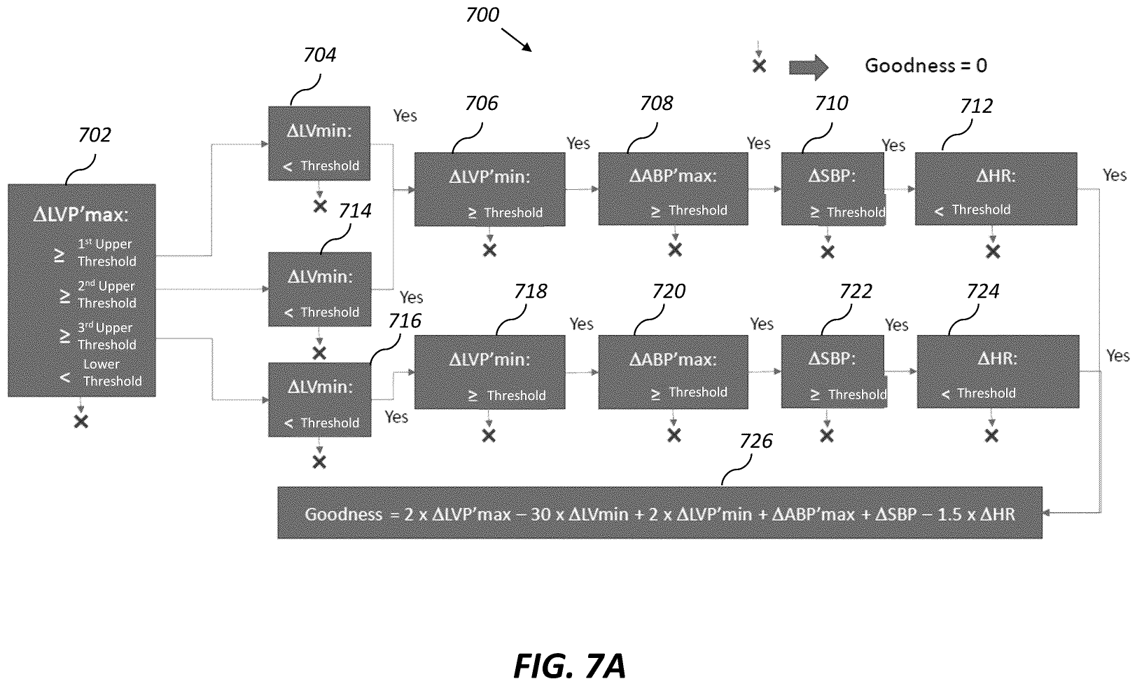

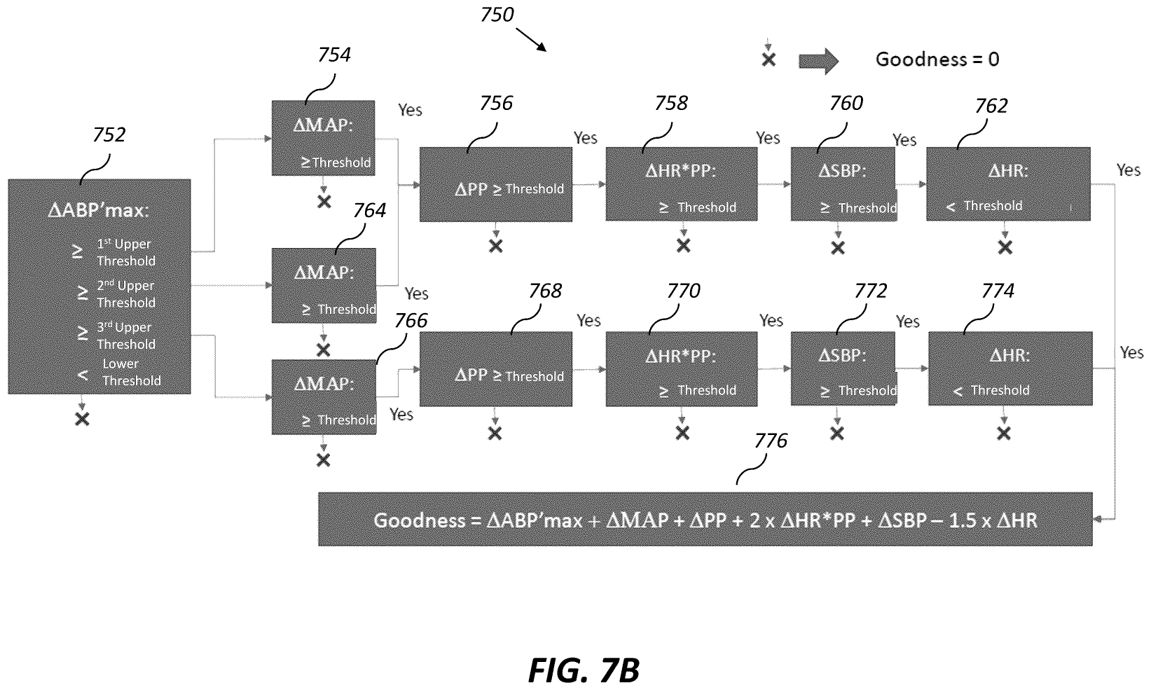

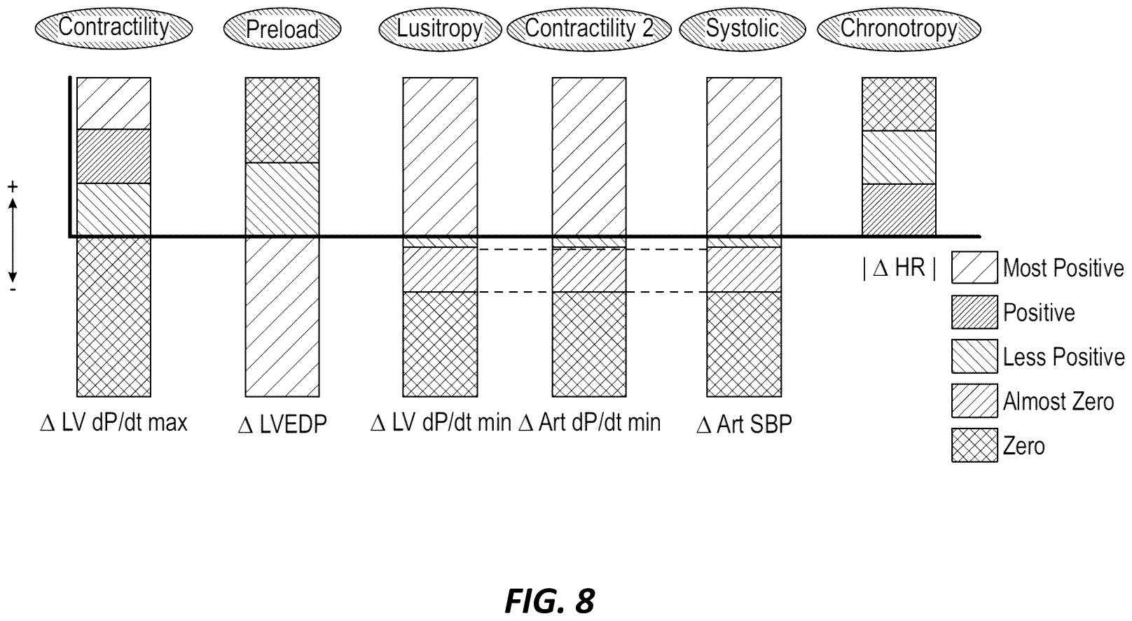

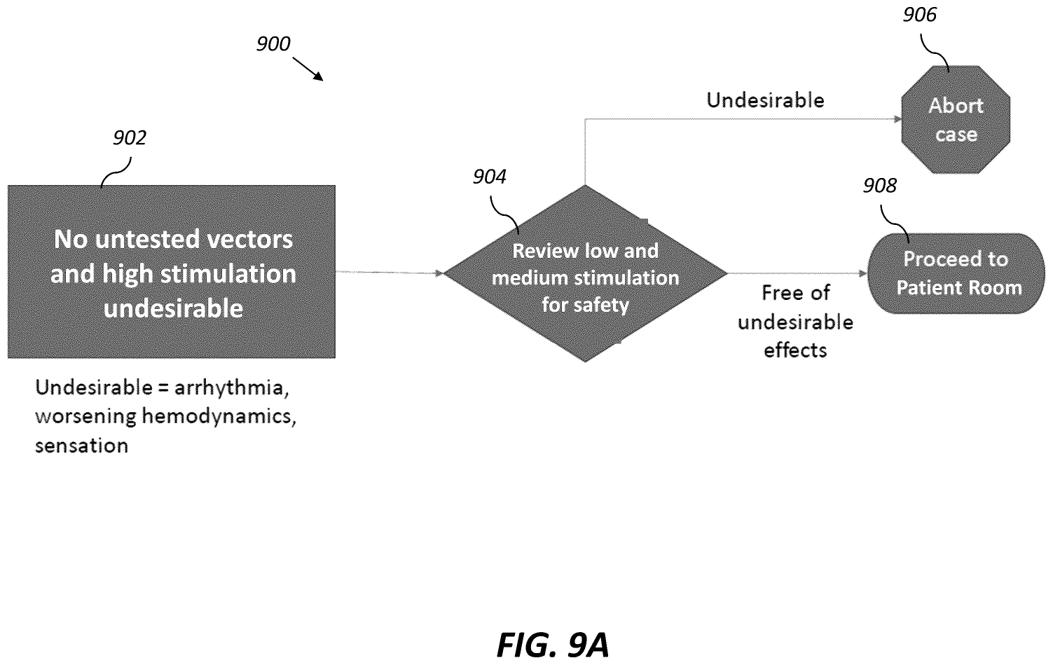

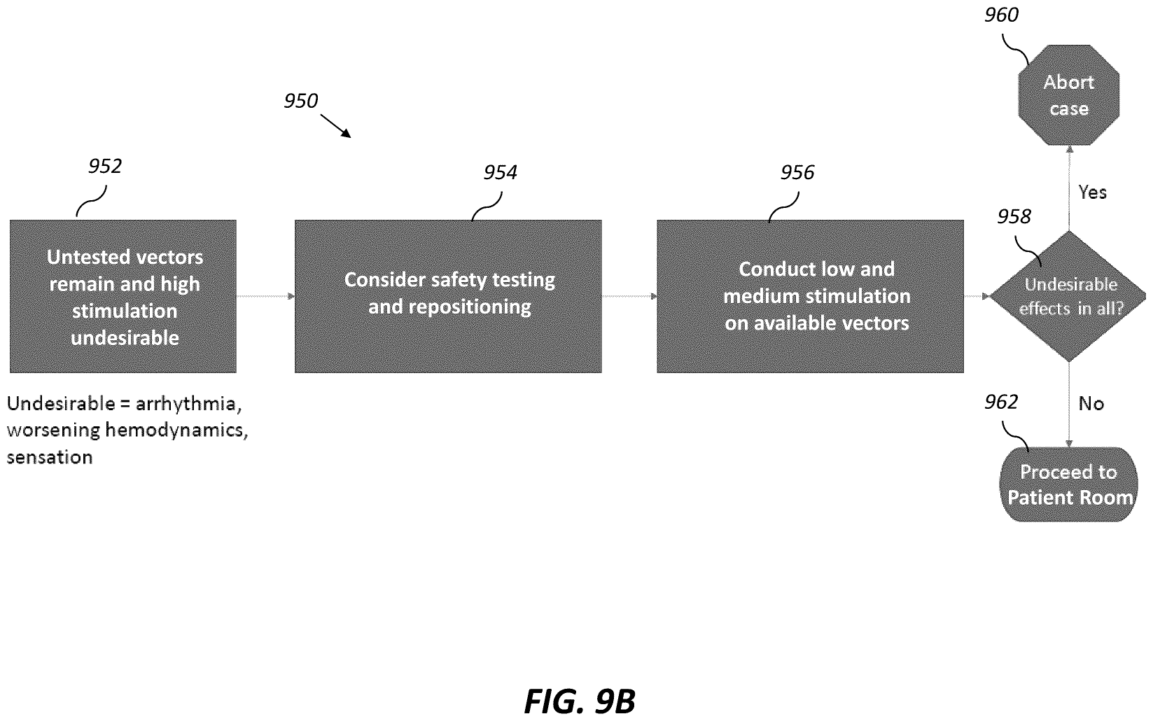

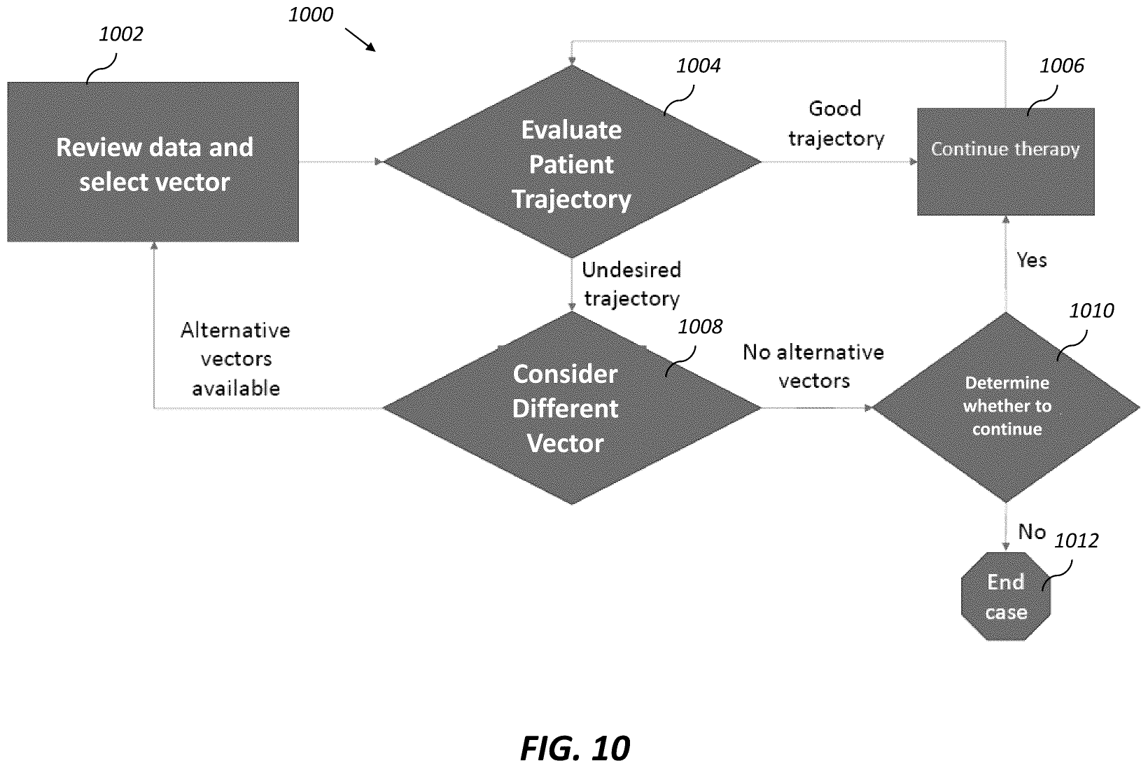

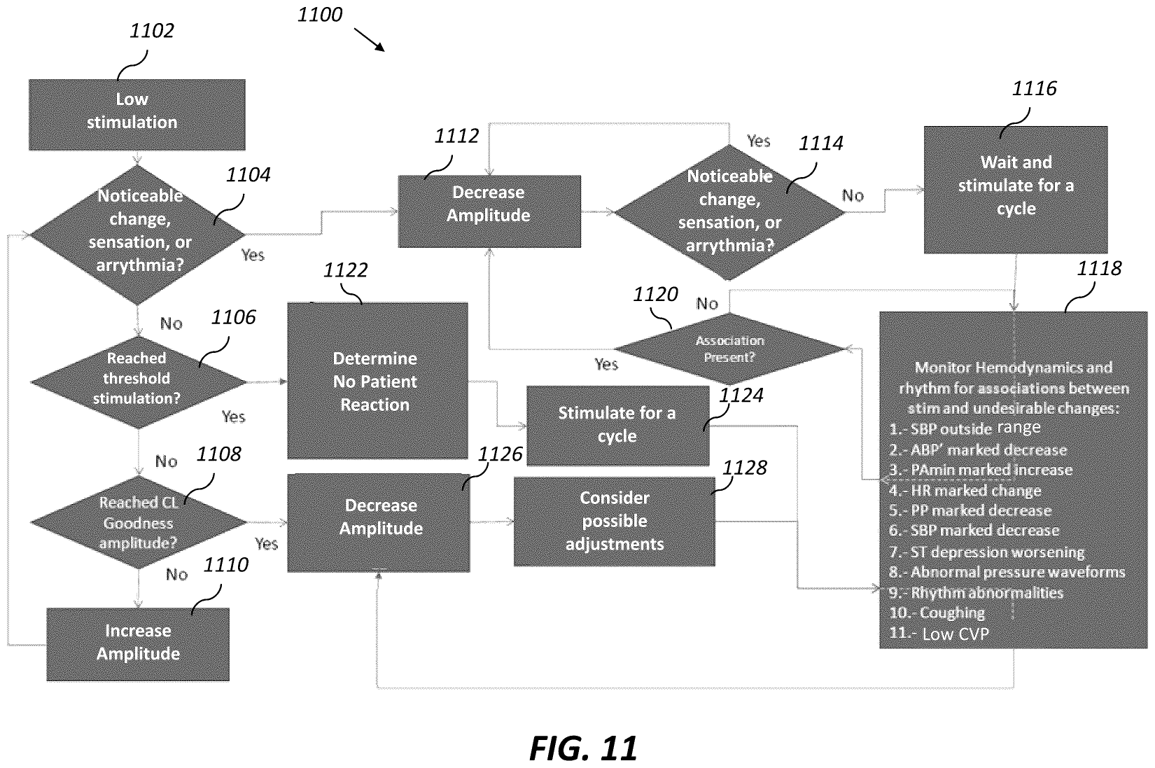

FIG. 2J is a schematic illustration of vasculature and surrounding nerves.

FIG. 2K is another schematic illustration of a heart and surrounding nerves.

FIG. 2L illustrates an example stimulation device.

FIG. 3A is a side view of an example expandable structure of a catheter system in an expanded state.

FIG. 3B is a proximal perspective view of a plurality of the example electrode assemblies coupled to an example expandable structure.

FIG. 3Ci is a top, side, and proximal perspective view of another example electrode assembly.

FIG. 3Cii is a back, side, and proximal perspective view of the example electrode assembly.

FIG. 3Ciii is a bottom plan view of an example upper insulator and example electrodes of the example electrode assembly.

FIG. 3Civ is a side view of an example upper insulator and example electrodes of the example electrode assembly.

FIG. 3D illustrates an example expandable device for positioning in a cavity of a subject.

FIG. 3E is an example schematic of electrodes that may be used in the method of FIGS. 3Fi-3Fviii.

FIGS. 3Fi-3Fviii illustrate an example method for selecting electrodes for stimulation.

FIG. 4 is a flowchart of an example of an optimization method for stimulation therapy.

FIG. 5 illustrates an example of staged atrial capture (AC) tests with vector tests.

FIG. 6A shows an example of a decision diagram for a first test of a vector test.

FIG. 6B shows an example of a decision diagram for a second test of a vector test.

FIG. 6C shows an example of a decision diagram for a third test of a vector test.

FIG. 6D shows an example of a decision diagram for a next vector test.

FIG. 7A shows a flow diagram of an example for a goodness indicator using left ventricular pressure.

FIG. 7B shows a flow diagram of an example for a goodness indicator using arterial blood pressure.

FIG. 8 illustrates an example of a goodness calculation for the left ventricular pressure of a patient.

FIG. 9A shows a flow diagram of an example for a protocol for no time remaining when no untested vectors in the target zone remain.

FIG. 9B shows a flow diagram of an example for a protocol for no time remaining when untested vectors in the target zone remain.

FIG. 10 shows a flow diagram of an example for the intensive care unit considerations for vector selection.

FIG. 11 shows a flow diagram of an example for uptitration in the intensive care unit.

FIG. 12 shows a flow chart of an example of therapy weaning.

FIG. 13 illustrates an example of a stimulation system for use with the catheters or catheter systems of the present disclosure.

FIG. 14A is a plot of contractility versus stimulation.

FIG. 14B is another plot of contractility versus stimulation.

### DETAILED DESCRIPTION

Several examples of the present disclosure provide for systems and methods for optimizing or improving stimulation or other modulation of a neural target, including, for example, cardiac pulmonary nerve stimulation (CPNS) therapy. The stimulation may be provided by an acutely implanted neural stimulation system, a chronically implanted neural stimulation system, or a non-implantable neural stimulation system (e.g., transcutaneous neural stimulator). The systems and methods herein can include CPNS with therapy titration, a multifactor efficacy algorithm or protocol to facilitate therapy titration (e.g., a goodness indicator), and therapy weaning. Therapy titration can include using physiological measurements of contractility (e.g., left ventricle dP/dt max, arterial dP/dt max), preload (e.g., left ventricle end-diastolic pressure), lusitropy (e.g., left ventricle dP/dt min), systolic function (e.g., arterial systolic blood pressure) and/or chronotropy (e.g., heart rate) to determine therapy efficacy (or likelihood thereof) and adjust therapy delivery (e.g., location, configuration and/or parameters of therapy). For example, the electrode position and stimulation intensity may be adjusted based on the physiological measurements, either automatically or by user control. Therapy titration can include closed-loop therapy delivery executed by one or more processors (e.g., one or more processors of a neuromodulation device (e.g., neural stimulation device including a pulse generator, one or more processors and memory), in which therapy is automatically adjusted based on the physiological measurements. A goodness indicator or other therapy optimization algorithm or protocol can be used to simultaneously monitor cardiovascular parameters such as contractility, preload, lusitropy, systolic function, and/or chronotropy. The goodness indicator or other therapy optimization algorithm or protocol can determine whether the change in these parameters from nerve stimulation is positive (e.g., beneficial or likely to be clinically effective), and further indicate optimal stimulation (e.g., location and configuration of stimulation elements such as electrodes and/or stimulation parameters). Therapy weaning can include gradually reducing stimulation while ensuring no significant adverse effects ensue. This can allow the patient or subject to maintain a therapeutic response to reduced CPNS therapy.

In accordance with several embodiments, the goal of CPNS can be to force a restart of the local control circuit of the intracardiac nervous system (ICNS) through stimulation. For example, stimulation can target the cardiac plexus at the level of the right pulmonary artery. The ICNS can include a network of intracardiac ganglia and interconnecting neurons that regulate cardiac activity. Dysfunction of the ICNS can cause heart diseases such as arrythmias. ICNS function can be rehabilitated by forcing it to restart and determining that its regulation is benefitting the patient. Different states of the ICNS can cause different patient outcomes. Stimulation with specific parameters can shift the ICNS into different such states.

CPNS therapy can trigger the ICNS to signal to the cardiac autonomic nervous system (CANS) that it is receiving excessive sympathetic stimulation. The systems and methods herein can include selecting a stimulation vector that sets the ICNS to a state that improves hemodynamics, and therefore cerebral blood flow. The systems and methods herein, combined or in isolation, can prompt the cardiac nervous system to reassess its current working state and allow the system to shift from the current maladaptive state (which can cause acute decompensated heart failure) to a virtuous cycle (which can improve the patient even after stimulation ends). By testing at different stimulation amplitudes and vectors, an optimal state can be reached.

Electrodes can be used to apply electrical neuromodulation to one or more nerves in and around the heart of a subject (e.g., patient). Several examples, for example, may be useful in electrical neuromodulation of patients with cardiovascular medical conditions, such as patients with acute or chronic cardiac disease. As discussed herein, several examples can allow for a portion of a catheter to be positioned within the vasculature of the patient in at least one of the right pulmonary artery, the left pulmonary artery, and the pulmonary trunk. Once positioned, an electrode system of the catheter can provide electrical energy (e.g., electrical current or electrical pulses) to stimulate the autonomic nervous system surrounding (e.g., proximate to) the pulmonary artery in an effort to provide adjuvant cardiac therapy to the patient. Sensed heart activity properties (e.g., non-electrical heart activity properties) can be used as the basis for making adjustments to one or more properties of the one or more electrical pulses delivered through the catheter positioned in the pulmonary artery of the heart in an effort to provide adjuvant cardiac therapy to the patient.

Certain groups of figures showing similar items follow a numbering convention in which the first digit or digits correspond to the drawing figure number and the remaining digits identify an element or component in the drawing. Similar elements or components between such groups of figures may be identified by the use of similar digits. For example, 336 may reference element “36” in FIG. 3A, and a similar element “36” may be referenced as 436 in FIG. 4. As will be appreciated, elements shown in the various examples herein can be added, exchanged, and/or eliminated so as to provide any number of additional examples of the present disclosure. Components or features described in connection with a previous figure may not be described in detail in connection with subsequent figures; however, the examples illustrated in the subsequent figures may include any of the components or combinations of components or features of the previous examples.

The terms “distal” and “proximal” are used herein with respect to a position or direction relative to the treating clinician taken along the devices of the present disclosure. “Distal” or “distally” are a position distant from or in a direction away from the clinician taken along the catheter. “Proximal” and “proximally” are a position near or in a direction toward the clinician taken along the catheter.

The catheter and electrode systems of the present disclosure can be used to treat a patient with various cardiac conditions. Such cardiac conditions include, but are not limited to, acute heart failure, among others. Several examples of the present disclosure provides methods that can be used to treat acute heart failure, also known as decompensated heart failure, by modulating the autonomic nervous system surrounding the pulmonary artery (e.g., the right pulmonary artery, the left pulmonary artery, the pulmonary trunk) in an effort to provide adjuvant cardiac therapy to the patient. The neuromodulation treatment can help by affecting autonomic function or balance (e.g., heart contractility and/or relaxation), in some examples more than heart rate. The autonomic nervous system may be modulated so as to collectively affect autonomic function or balance (e.g., heart contractility and/or relaxation), in some examples more than heart rate. The autonomic nervous system can be impacted by electrical modulation that includes stimulating and/or inhibiting nerve fibers of the autonomic nervous system.

As discussed herein, the one or more electrodes present on the catheter can be positioned within the main pulmonary artery and/or one or both of the right and left pulmonary arteries. In accordance with several examples, the one or more electrodes are positioned in contact the luminal surface of the main pulmonary artery, and/or right or left pulmonary artery (e.g., in physical contact with the surface of the posterior portion of the main pulmonary artery). As will be discussed herein, the one or more electrodes on the catheter and/or catheter system provided herein can be used to provide pulse of electrical energy between the electrodes and/or the reference electrodes. The electrodes of the present disclosure can be used in any one of a unipolar, bi-polar and/or a multi-polar configuration. Once positioned, the catheter and the catheter system of the present disclosure can provide the stimulation electrical energy to stimulate the nerve fibers (e.g., autonomic nerve fibers) surrounding the main pulmonary artery and/or one or both of the right and left pulmonary arteries in an effort to provide adjuvant cardiac therapy to the patient (e.g., electrical cardiac neuromodulation).

In some examples, systems other than intravascular catheters may be used in accordance with the methods described herein. For example, electrodes, sensors, and the like may be implanted during open heart surgery or without being routed through vasculature.

Several examples, as will be discussed more fully herein, may allow for the electrical neuromodulation of the heart of the patient that includes delivering one or more electrical pulses through a catheter positioned in a pulmonary artery of the heart of the patient, sensing from at least a first sensor positioned at a first location within the vasculature of the heart one or more heart activity properties (e.g., non-electrical heart activity properties) in response to the one or more electrical pulses, and adjusting a property of the one or more electrical pulses delivered through the catheter positioned in the pulmonary artery of the heart in response to the one or more heart activity properties in an effort to provide adjuvant cardiac therapy to the patient.

The catheter can include a plurality of electrodes, which are optionally inserted into the pulmonary trunk, and positioned such that the electrodes are, preferably, in contact with the posterior surface, the superior surface, and/or the inferior surface of the pulmonary artery. From such locations, electrical pulses can be delivered to or from the electrodes to selectively modulate the autonomic nervous system of the heart. For example, electrical pulses can be delivered to or from one or more of the electrodes to selectively modulate the autonomic cardiopulmonary nerves of the autonomic nervous system, which can modulate autonomic function or balance (e.g., heart contractility and/or relaxation), in some examples more than heart rate. Preferably, the plurality of electrodes is positioned at a site along the posterior wall and/or superior wall of the pulmonary artery, for example the right or left pulmonary artery. From such a position in the pulmonary artery, one or more electrical pulses can be delivered through the electrodes and one or more heart activity properties (e.g., non-electrical heart activity properties) can be sensed. Based at least in part on these sensed heart activity properties, a property of the one or more electrical pulses delivered to or from the electrodes positioned in the pulmonary artery of the heart can be adjusted in an effort to positively influence autonomic function or balance (e.g., heart contractility and/or relaxation) while reducing or minimizing the effect on heart rate and/or oxygen consumption. In certain examples, the effect on heart contractility is to increase heart contractility. In certain examples, the effect on heart relaxation is to increase heart relaxation.

### Example Devices

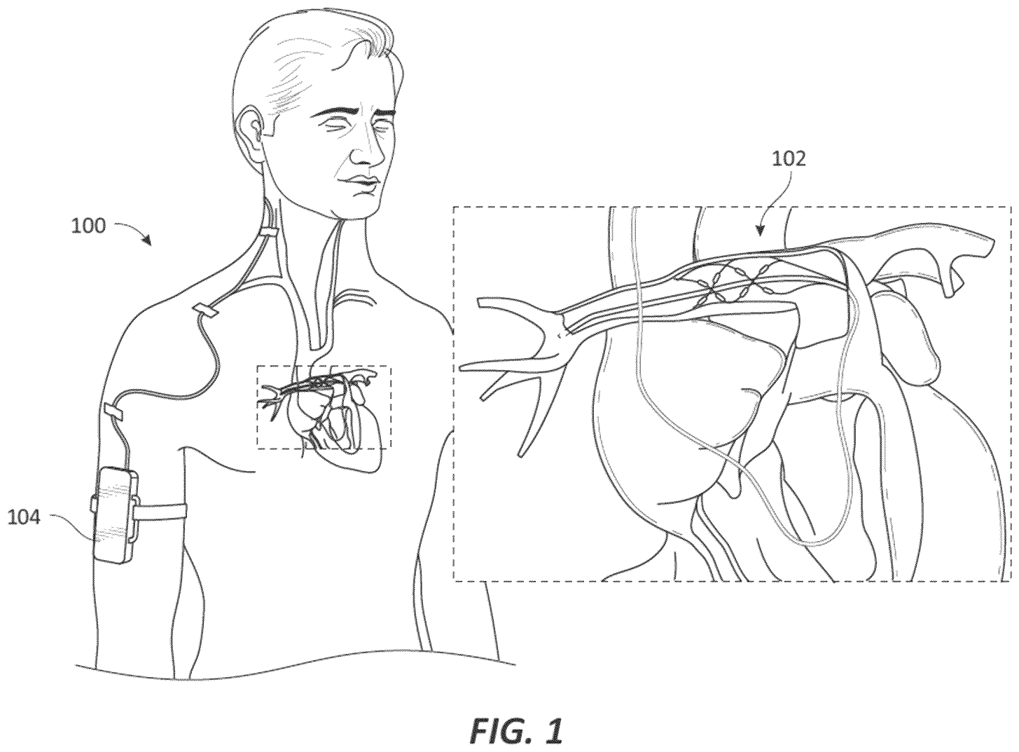

FIG. 1 schematically illustrates a system 100 that can be used to apply electrical neuromodulation to tissue (e.g., including one or more nerves) in and around the heart of a subject. The system 100 can be used to provide CPNS therapy. The system 100 comprises a first component 102 and a second component 104. The first component 102 may be positioned in a pulmonary artery (e.g., the right pulmonary artery as shown in FIG. 1, the left pulmonary artery, and/or the pulmonary trunk). The first component 102 may be endovascularly positioned via a minimally invasive, transdermal, percutaneous procedure, for example routed through the vasculature from a remote location such as a jugular vein (e.g., an internal jugular vein, as shown in FIG. 1), an axial subclavian vein, a femoral vein, or other blood vessels. Such an approach can be over-the-wire, using a Swan-Ganz float catheter, combinations thereof, etc. In some examples, the first component may be positioned invasively, for example during conventional surgery (e.g., open-heart surgery), placement of another device (e.g., coronary bypass, pacemaker, defibrillator, etc.), or as a stand-alone procedure. As described in further detail herein, the first component comprises a neuromodulator (e.g., one or more electrodes, one or more ultrasound transducers, one or more drugs, one or more ablation devices, one or more microwave-emitting elements, one or more laser devices, one or more cryo devices, combinations thereof, and the like) and may optionally comprise a stent or framework, an anchoring system, and/or other components, such as the examples shown and described in connection with FIGS. 3A-3D. The first component 102 may be acutely positioned in the pulmonary artery for 24 to 72 hours. In some implementations, the first component 102 may be chronically implanted for a longer period of time. In some examples, the first component 102 neuromodulates terminal branches within the cardiac plexus, which can increase left ventricle contractility and/or relaxation and/or otherwise improve autonomic balance or function. The increase in left ventricle contractility and/or relaxation may be without an increase in heart rate or may be greater than (e.g., based on a percentage change) than an increase in heart rate. In some examples, the first component 102 may be adapted to ablate tissue, including nerves, in addition to or instead of modulating tissue such as nerves. In some examples, the first component 102 may not be inserted within the subject and may instead deliver therapy transcutaneously.

The first component 102 is electrically coupled to the second component 104 (e.g., via wires or conductive elements routed via a catheter, for example as illustrated in FIG. 1, and/or wirelessly). The second component 104 may be positioned extracorporeally (e.g., strapped to a subject's arm as shown in FIG. 1, strapped to another part of the subject (e.g., leg, neck, chest), placed on a bedside stand, etc.). In some examples, the second component 104 may be temporarily implanted in the subject (e.g., in a blood vessel, in another body cavity, in a chest, etc.). The second component 104 includes electronics (e.g., pulse generator) configured to operate the one or more electrodes (or other neuromodulator(s)) in the first component 102. The second component 104 may include a power supply or may receive power from an external source (e.g., a wall plug, a separate battery, etc.). The second component 104 may include electronics configured to receive sensor data from one or more sensors.

The system 100 may comprise one or more sensors. The sensor(s) may be positioned in one or more of a pulmonary artery (e.g., right pulmonary artery, left pulmonary artery, and/or pulmonary trunk), an atrium (e.g., right and/or left), a ventricle (e.g., right and/or left), a vena cava (e.g., superior vena cava and/or inferior vena cava), and/or other cardiovascular locations. The sensor(s) may be part of the first component 102, part of a catheter, and/or separate from the first component 102 (e.g., electrocardiogram chest monitor, pulse oximeter, etc.). The sensor(s) may be in communication with the second component 104 (e.g., wired and/or wireless). The second component 104 may initiate, adjust, calibrate, cease, etc. neuromodulation based on information from the sensor(s).

The system 100 may comprise an “all-in-one” system in which the first component 102 is integral or monolithic with the targeting catheter. For example, the first component 102 may be part of a catheter that is inserted into an internal jugular vein, an axial subclavian vein, a femoral vein, etc. and navigated to a target location such as the pulmonary artery. The first component 102 may then be deployed from the catheter. Such a system can reduce the number and/or complexity of procedural steps and catheter exchanges used to position the first component 102. For example, a guidewire may be at least twice as long as a target catheter, which can be difficult to control in a sterile field. Such a system may make repositioning of the first component 102 easier after an initial deployment because positioning systems are already in place.

The system 100 may comprise a telescoping and/or over-the-wire system in which the first component 102 is different than the targeting catheter. For example, a targeting catheter (e.g., a Swan-Ganz catheter) may be inserted into an internal jugular vein, an axial subclavian vein, a femoral vein, etc. and navigated to a target location such as the pulmonary artery (e.g., by floating). A guidewire may be inserted into a proximal hub through the target catheter to the target location (e.g., having a stiffest portion exiting the target catheter distal end) and the first component 102 as part of a separate catheter than the target catheter may be tracked to the target location over the guidewire or using telescoping systems such as other guidewires, guide catheters, etc. The first component 102 may then be deployed from the separate catheter. Such systems are known by interventional cardiologists such that multiple exchanges may be of little issue. Such a system may allow customization of certain specific functions. Such a system may reduce overall catheter diameters, which can increase trackability, and/or allow additional features to be added, for example because not all functions are integrated into one catheter. Such a system may allow use of multiple catheters (e.g., removing a first separate catheter and positioning a second separate catheter without having to reposition the entire system). For example, catheters with different types of sensors may be positioned and removed as desired. The system 100 may be steerable (e.g., comprising a steerable catheter) without a Swan-Ganz tip. Some systems 100 may be compatible with one or more of the described types of systems (e.g., a steerable catheter with an optionally inflatable balloon for Swan-Ganz float, a steerable catheter that can be telescoped over a guidewire and/or through a catheter, etc.).

FIG. 2A schematically illustrates a heart 200 and surrounding areas. The main pulmonary artery or pulmonary trunk 202 begins at the outlet of the right ventricle 204. In an adult, the pulmonary trunk 202 is a tubular structure having a diameter of about 3 centimeter (cm) (approx. 1.2 inches (in)) and a length of about 5 (approx. 2.0 in). The main pulmonary artery 202 branches into the right pulmonary artery 206 and the left pulmonary artery 208, which deliver deoxygenated blood to the corresponding lung. As illustrated in FIG. 2A, the main pulmonary artery 202 has a posterior surface 210 that arches over the left atrium 212 and is adjacent to the pulmonary vein 213. As discussed herein, a neurostimulator can be positioned at least partially in a pulmonary artery 202, 206, 208, for example with the neurostimulator in contact with the posterior surface 210. In some examples, a preferred location for positioning the neurostimulator is the right pulmonary artery 206. The electrode array can be positioned between the branch point defining the left pulmonary artery 208 and the right pulmonary artery 206 and the branch point that divides the right pulmonary artery 206 into at least two additional arteries. The neurostimulator can provide CPNS therapy by stimulating the cardiac plexus of the right pulmonary artery 206, which can force a restart of the local control circuit of the intracardiac nervous system (ICNS). In some examples, a preferred location for positioning the neurostimulator is in contact with the posterior surface 210 of the pulmonary artery 202, 206, 208. From such a location, stimulation electrical energy delivered from an electrode, for example, may be better able to treat and/or provide therapy (including adjuvant therapy) to a subject experiencing a variety of cardiovascular medical conditions, such as acute heart failure. Other locations for the neurostimulator in the pulmonary artery 202, 206, 208 or in other anatomical locations are also possible.

The first component 102 (FIG. 1) can be positioned in the pulmonary artery 202, 206, 208 of the subject, where the neurostimulator of the first component 102 is in contact with the luminal surface of the pulmonary artery 202, 206, 208 (e.g., in physical contact with or proximate to the surface of the posterior portion 210 of the pulmonary artery 202, 206, 208). The neurostimulator of the first component 102 can be used to deliver the stimulation to the autonomic cardiopulmonary fibers surrounding the pulmonary artery 202, 206, 208. The stimulation electrical energy can elicit responses from the autonomic nervous system that may help to modulate a subject's autonomic function or balance (e.g., cardiac contractility and/or relaxation). The stimulation may affect contractility and/or relaxation more than the heart rate, which can improve hemodynamic control while possibly reducing unwanted systemic effects.

In some examples, neuromodulation of targeted nerves or tissue as described herein can be used for the treatment of arrhythmia, atrial fibrillation or flutter, diabetes, eating disorders, endocrine diseases, genetic metabolic syndromes, hyperglycemia (including glucose tolerance), hyperlipidemia, hypertension, inflammatory diseases, insulin resistance, metabolic diseases, obesity, ventricular tachycardia, conditions affecting the heart, and/or combinations thereof.

FIGS. 2B-2D are schematic illustrations of a heart 200 and surrounding areas from various perspectives. Portions of the heart 200 (e.g., the aorta, the superior vena cava, among other structures), including a portion of the pulmonary trunk 202, have been removed to allow for the details discussed herein to be shown. FIG. 2B provides a perspective view of the heart 200 as seen from the front of the subject or patient (viewed in an anterior to posterior direction), while FIG. 2C provides a perspective view of the heart 200 as seen from the right side of the subject. As illustrated, the heart 200 includes the pulmonary trunk 202 that begins at the base of the right ventricle 204. In an adult, the pulmonary trunk 202 is a tubular structure approximately 3 centimeters (cm) in diameter and 5 cm in length. The pulmonary trunk 202 branches into the right pulmonary artery 206 and the left pulmonary artery 208 at a branch point or bifurcation 207. The left pulmonary artery 106 and the right pulmonary artery 108 serve to deliver de-oxygenated blood to each corresponding lung.

The branch point 207 includes a ridge 209 that extends from the posterior of the pulmonary trunk 202. As illustrated, the branch point 207, along with the ridge 209, provides a “Y” or “T” shaped structure that helps to define at least a portion of the left pulmonary artery 208 and the right pulmonary artery 206. For example, from the ridge 209, the branch point 207 of the pulmonary trunk 202 slopes in opposite directions. In a first direction, the pulmonary trunk 202 transitions into the left pulmonary artery 208, and in the second direction, opposite the first direction, the pulmonary trunk 202 transitions into the right pulmonary artery 206. The branch point 207 may not necessarily be aligned along a longitudinal center line 214 of the pulmonary trunk 202.

As illustrated in FIG. 2B, portions of the pulmonary artery 202 can be defined with a right lateral plane 216 that passes along a right luminal surface 218 of the pulmonary trunk 202, a left lateral plane 220 parallel with the right lateral plane 216, where the left lateral plane 220 passes along a left luminal surface 222 of the pulmonary trunk 202. The right lateral plane 216 and the left lateral plane 220 extend in both a posterior direction 224 and anterior direction 226. As illustrated, the ridge 209 of the branch point 207 is located between the right lateral plane 216 and the left lateral plane 220. The branch point 207 is positioned between the right lateral plane 216 and the left lateral plane 220, where the branch point 207 can help to at least partially define the beginning of the left pulmonary artery 208 and the right pulmonary artery 206 of the heart 200. The distance between the right lateral plane 216 and the left lateral plane 220 is approximately the diameter of the pulmonary trunk 202 (e.g., about 3 cm).

As discussed herein, the present disclosure includes methods for neuromodulation of the heart 200 of a subject or patient. For example, as discussed herein, a catheter positioned in the pulmonary artery 202 can be used to deliver one or more electrical pulses to the heart 200. A first sensor, for example as discussed herein, positioned at a first location within the vasculature of the heart 200, senses a heart activity property in response to the neurostimulation. Properties of the neurostimulator can be adjusted in response to the sensed heart activity property in an effort to provide adjuvant cardiac therapy to the patient.

FIG. 2D provides an additional illustration of the posterior surface 221, the superior surface 223, and the inferior surface 225 of the right pulmonary artery 206. As illustrated, the view of the heart 200 in FIG. 2D is from the right side of the heart 200. As illustrated, the posterior surface 221, the superior surface 223, and the inferior surface 225 account for approximately three quarters of the luminal perimeter of the right pulmonary artery 206, where the anterior surface 227 accounts for the remainder. In some implementations, electrodes of a neurostimulation device may be positioned adjacent to the anterior surface 227. The electrodes of the neurostimulation device may span a portion of a circumference, and the portion may span (e.g., only span) or be configured to span, for example, the anterior surface 227 and/or the superior surface 223. The electrodes may cover or span between about 10% and about 50% (e.g., about 10%, about 15%, about 20%, about 25%, about 30%, about 35%, about 40%, about 45%, about 50%, ranges between such values, etc.) of a circumference of the device and/or artery (e.g., the anterior surface 227 and/or the superior surface 223). The electrodes may cover or span between about 10 mm and about 40 mm (e.g., about 10 mm, about 15 mm, about 20 mm, about 25 mm, about 30 mm, about 35 mm, about 40 mm, ranges between such values, etc.) of a circumference of the device and/or artery (e.g., the anterior surface 227 and/or the superior surface 223). In certain such implementations, electrodes of the neurostimulation device may also or alternatively be positioned adjacent to the superior surface 223. FIG. 2D also illustrates the aorta 230, pulmonary veins 213, the superior vena cava (SVC) 232, and the inferior vena cava (IVC) 234.

FIGS. 2E and 2F are schematic illustrations of a heart 200 and surrounding nerves. The cardiovascular system is richly innervated with autonomic fibers. Sympathetic fibers originate from stellate and thoracic sympathetic ganglia, and are responsible for increases in the chronotropic (heart rate), lusotropic (relaxation), and inotropic (contractility) state of the heart. Human cadaver anatomical studies show that the fibers responsible for the lusotropic and inotropic state of the ventricles pass along the posterior surface of the right pulmonary artery 206 and the pulmonary trunk 202. FIG. 2E illustrates approximate positions of the right dorsal medial common peroneal nerve (CPN) 240, the right dorsal lateral CPN 242, the right stellate CPN 244, the right vagal nerve or vagus 246, the right cranial vagal CPN 248, the right caudal vagal CPN 250, the right coronary cardiac nerve 252, the left coronary cardiac nerve 254, the left lateral cardiac nerve 256, the left recurrent laryngeal nerve 258, the left vagal nerve or vagus 260, the left stellate CPN 262, the left dorsal lateral CPN 264, and the left dorsal medial CPN 266. These and/or other nerves surrounding (e.g., proximate to) the heart 200 can be targeted for neurostimulation by the systems and methods described herein. In some examples, at least one of the right dorsal medial common peroneal nerve 240, the right stellate CPN 244, and the left lateral cardiac nerve 256 is targeted and/or affected for neuromodulation, although other nerves, shown in FIG. 2E or otherwise, may also be targeted and/or affected.

FIGS. 2E and 2F also schematically illustrate the trachea 241. As best seen in FIG. 2F, the trachea 241 bifurcates into the right pulmonary bronchus 243 and the left pulmonary bronchus 245. The bifurcation of the trachea 241 can be considered along a plane 247. The plane 247 is along the right pulmonary artery 206. The bifurcation of the pulmonary artery can be considered along a plane 247, which is spaced from the plane 247 by a gap 249. The gap 249 spans the right pulmonary artery 206. A large number of cardiac nerves cross the right pulmonary artery 206 along the gap 249 as illustrated by the circled area 251, and these nerves may be advantageously targeted by some of the systems and methods described herein. In certain such examples, the bifurcation of the trachea 241 and/or the bifurcation of the pulmonary artery 202 may provide a landmark for system and/or component positioning. Stimulation electrodes may be spaced from the trachea 241, for example to reduce cough or other possible respiratory side effects. In some examples, stimulation electrodes are spaced from the trachea 241 or the plane 247 by between about 2 mm and about 8 mm (e.g., about 2 mm, about 3 mm, about 4 mm, about 5 mm, about 6 mm, about 7 mm, about 8 mm, ranges between such values, etc.). In some examples, stimulation electrodes are spaced from the trachea 241 or the plane 247 by a percentage of a length of the right pulmonary artery 206 between about 10% and about 100% (e.g., about 10%, about 25%, about 30%, about 35%, about 40%, about 45%, about 50%, about 55%, about 75%, about 100%, ranges between such values, etc.).

FIGS. 2G and 2H are schematic illustrations of vasculature and an electrode matrix 201. A majority of the electrode matrix 201 is positioned in the right pulmonary artery 206, although some of the electrode matrix 201 may be considered positioned in the pulmonary trunk 202. The electrode array is shown as a 4×5 matrix of electrodes 203. As described in further detail herein, the electrodes 203 may be positioned on splines, positioned on a membrane or mesh coupled to splines, etc. For example, four splines may each contain five electrodes 203. In some examples, the electrodes 203 comprise bipolar electrodes with controllable polarity, allowing configurability of the electrode matrix 201. In some examples, edge-to-edge spacing of the electrodes 203 is between about 3 mm and about 7 mm (e.g., about 3 mm, about 4 mm, about 5 mm, about 6 mm, about 7 mm, ranges between such values, etc.). In some examples, the electrodes 203 have a surface area between about 0.5 mm² and about 5 mm² (e.g., about 0.5 mm², about 1 mm², about 1.5 mm², about 2 mm², about 2.5 mm², about 3 mm², about 3.5 mm², about 4 mm², about 4.5 mm², about 5 mm², ranges between such values, etc.). The electrodes 203 are generally aligned longitudinally and circumferentially, but offset electrodes 203 are also possible. The coverage of the right pulmonary artery 206 provided by the electrode array 201 is longitudinally between about 25 mm and about 35 mm (e.g., about 25 mm, about 28 mm, about 31 mm, about 35 mm, ranges between such values, etc.) and is circumferentially between about 80° and about 1200 (e.g., about 80°, about 90°, about 100°, about 110°, about 120°, ranges between such values, etc.). The electrode array 201 may cover, for example, between about 25% and about 50% (e.g., about 25%, about 30%, about 35%, about 40%, about 45%, about 50%, ranges between such values, etc.) of the circumference of the vessel. In some examples, the electrode array 201 comprises a 3×3 matrix, a 3×4 matrix, a 3×5 matrix, a 4×4 matrix, a 4×5 matrix, or a 5×5 matrix. Larger matrices may be more likely to capture the target nerve by at least one combination of electrodes 203, and smaller matrices may be easier to deliver to the target site. Referring again to FIG. 2D, in some implementations, an electrode array having the features described herein may be positioned adjacent to the anterior surface 227. In certain such implementations, the electrode array may also or alternatively be positioned adjacent to the superior surface 223.

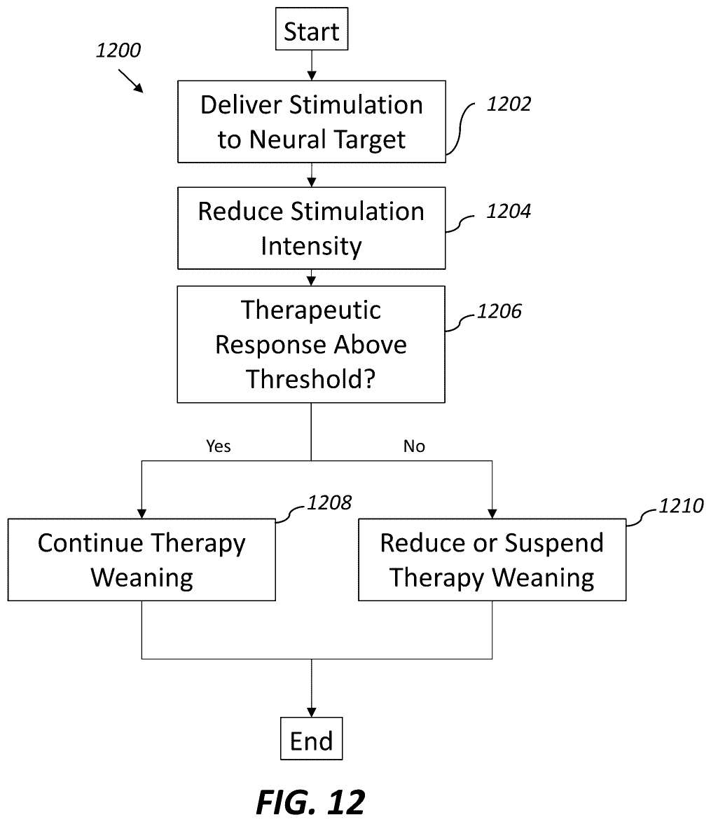

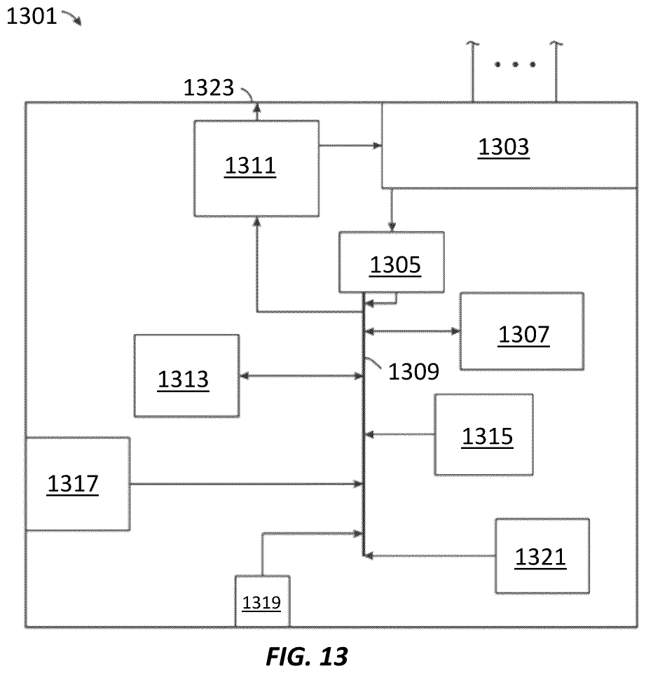

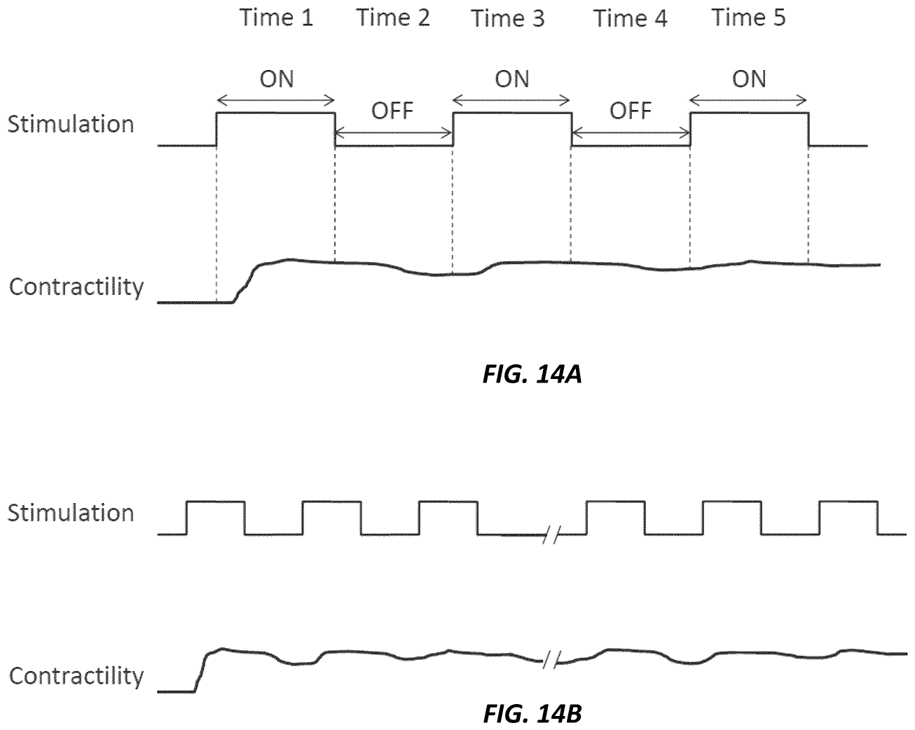

FIG. 21 is a schematic illustration of heart vasculature and surrounding nerves. Similar to FIGS. 2G and 2H, FIG. 21 shows a pulmonary trunk 202, a right pulmonary artery 206, and a left pulmonary artery 208. FIG. 21 also shows traces of the approximate crossing locations of interventricular sulcus nerves 215, 217 along the right pulmonary artery 206 and the pulmonary trunk 202. Stimulation of one or both of the nerves 215, 217 may increase autonomic function or balance (e.g., contractility and/or relaxation), for example more than heart rate or without affecting heart rate. The electrode matrix 201, including electrodes 203a, 203b, 203c, 203d, 203e, 203f, etc., is shown in phantom in the approximate position of FIGS. 2G and 2H.

In some examples, particular electrodes can be selected to target or capture one or more nerves. The electrodes 203a, 203b can be used to target the nerve 215, for example, in a generally transverse manner. The electrodes 203a, 203c can be used to target the nerve 215, for example, in a generally parallel manner. The electrodes 203c, 203d can be used to target the nerve 215 as well as the nerve 217, for example, in a generally transverse manner. The electrodes 203e, 203f can be used to target the nerve 217, for example, in a generally mixed transverse-parallel manner. In some examples, the two electrodes can be used in a bipolar manner, with one of the two electrodes being positive and the other of the two electrodes being negative. In some examples, more than two electrodes can be used, with two or more electrodes being positive and two or more electrodes being negative.

As described in further detail herein, upon placement of the electrode array, electrode combinations can be stimulated to test their effect. Some combinations may produce a better result but be more likely to result in a side effect, some combinations may produce a better result but be less repeatable, some combinations may affect one nerve but not multiple nerves, etc. In some examples, a plurality of electrode combinations or independent outputs can be used in parallel or in series. For example, the electrodes 203a, 203b can be used to target the nerve 215 for a first duration and the electrodes 203e, 203f can be used to target the nerve 217 for a second duration. The second duration may at least partially overlap the first duration, fully overlap the first duration (e.g., starting at the same time, ending at the same time, starting after the first duration starts, ending before the first duration ends, and combinations thereof) or may be temporally spaced from the first duration by a third duration. The third duration may be zero (e.g., the second duration starting as the first duration ends).

In a study of multiple cadavers, the mean diameter 206d of the right pulmonary artery 206 proximate to the branch point 207 was about 26.5 mm with a standard deviation of about 4.6 mm. Assuming a circular vessel, the mean circumference of the right pulmonary artery 206 proximate to the branch point 207 is about 83 mm. If the goal is 30% coverage of the circumference, then an electrode matrix should have a circumferential length of about 25 mm (83 mm×30%). Other electrode matrix dimensions can be estimated or calculated based on other dimensions (e.g., vessel diameter at other points, measured vessel diameter, diameters of other vessels, vessel lengths, etc.), target coverage percentage, nerve location variability, placement accuracy, stimulation parameters, etc.

FIG. 2J is a schematic illustration of vasculature and surrounding nerves. The superior vena cava 232, as discussed above, supplies blood to the right atrium of the heart. The vessels supplying blood to the superior vena cava 232 include the right innominate vein or right brachiocephalic vein 253 and the left innominate vein or left brachiocephalic vein 255. The vessels supplying blood to the right brachiocephalic vein 253 include the right subclavian vein 257 and the right internal jugular vein 259. The vessels supplying blood to the left brachiocephalic vein 255 include the left subclavian vein 261 and the left internal jugular vein 263. The inferior thyroid vein 265 also supplies blood to the superior vena cava 232. Although other nerves are present surrounding the vasculature illustrated in FIG. 2F, the right vagus nerve 267 is illustrated as an example. The left vagus nerve runs close to the left internal jugular vein 263 and the common carotid artery, and then crosses the left brachiocephalic vein 255. Thoracic sympathetic cardiac branches also cross the left brachiocephalic vein 255 closer to the crown of the aorta and more medial, generally between the junction of the left subclavian vein and the left internal jugular vein 263 and about half of the length of the left brachiocephalic vein 253. Vasculature that may not typically be characterized as cardiovasculature may also be used in accordance with certain methods and systems described herein.

FIG. 2K is another schematic illustration of a heart 200 and surrounding nerves. As described in detail herein, nerves affecting autonomic function or balance (e.g., contractility and/or relaxation such as left ventricle contractility and/or relaxation) may be targeted for neuromodulation by positioning a catheter in the pulmonary artery (e.g., right pulmonary artery, pulmonary trunk, left pulmonary artery). In some examples, a nerve such as the right stellate CPN 244 may also or alternatively be targeted by positioning a device at a location 272 in the left subclavian artery 274 and/or the location 276 in the descending aorta 278. Positioning in the left common carotid artery 280 is also possible. In FIG. 2K, an example stimulation device 282 is shown at the locations 272, 276. Other stimulation devices are also possible. In examples comprising multiple stimulation devices, the stimulation devices may be the same, different, or similar (as a non-limiting example, having a same structure but different dimensions).

FIG. 2L illustrates an example stimulation device 282. The stimulation device 282 may be used, for example, to target stimulation of a right stellate CPN 244 or another nerve. The device 282 comprises a skeletal structure 284, for example a stent, hoops, etc. The skeletal structure 284 may comprise a shape memory material (e.g., nitinol) that is self-expanding. The device 282 further comprise a mesh or membrane 286 attached to the skeletal structure 284. The mesh 286 may comprise, for example, Dacron®. One side of the device 282 comprises an electrode array 288. The electrode array 288 may have an area between about 0.5 cm² and about 3 cm² (e.g., about 0.5 cm², about 1 cm², about 1.5 cm², about 2 cm², about 2.5 cm², about 3 cm², ranges between such values, etc.). The electrode array 288 may be powered by implantable electronics 290. The electronics 290 may include, for example, non-volatile memory (e.g., storing electrode combinations and parameters), ASIC stimulation engine and logic, RF engine, battery power, and a sensor (e.g., pressure sensor, contractility sensor, combinations thereof, etc.). The device 282 may be positioned by a catheter routed through vasculature (e.g., from a femoral or radial artery). The device 282 may be positionable until the target nerve is stimulated. In some examples, the electrode array 288 may be electronically repositionable. In some examples, an external device (e.g., worn by the subject) can power and/or control the device 282. In examples in which the electronics 290 can power and/or control the device 282, the device 282 may be fully implantable. In certain such examples, the device 282 may be combined with a pacemaker, defibrillator, or other implantable stimulation device.

FIG. 3A is a side view of an example expandable structure 320 of a catheter system in an expanded state. The system comprises a proximal portion configured to remain out of the body of a subject and a distal portion configured to be inserted into vasculature of a subject. The distal portion comprises an expandable structure 320. The system comprises an outer sheath an elongate inner member 308 radially inward of the outer sheath. The system may include a shaft radially inward of the inner member 308. The inner member 308 may comprise a guidewire lumen, for example allowing the system to be tracked over a guidewire. The shaft may comprise a guidewire lumen, for example allowing the system to be tracked over a guidewire. The outer sheath and the inner member 308 may be coupled at the proximal end.

The proximal portion may comprise a handle and an actuation mechanism, for example to move the outer sheath relative to the inner member 308. To deploy the expandable structure, the outer sheath may be retracted while the inner member 308 remains stationary, the inner member 308 may be advanced while the outer sheath remains stationary, and/or the outer sheath may be retracted while the inner member 308 is advanced. To collapse the expandable structure 320, the outer sheath may be advanced while the inner member 308 remains stationary, the inner member 308 may be retracted while the outer sheath remains stationary, and/or the outer sheath may be advanced while the inner member 308 is retracted. The proximal portion may comprise a handle and an actuation mechanism, for example to move the outer sheath relative to the shaft 303. The proximal portion may comprise a handle and an actuation mechanism, for example to move the inner member 308 relative to the shaft 303. The handle may include a locking mechanism, for example as described herein.

The sheath may comprise a reinforcing layer, for example a braid, a coil, a helix, combinations thereof, etc. The reinforcing layer may provide column strength to capture the expandable structure 320. A distal end of the sheath may be atraumatic. For example, after the expandable structure 320 is deployed, the distal end of the sheath being atraumatic can reduce or prevent injuries from interaction between the sheath and the vasculature. In some examples, the distal end of the sheath may be selectively positioned or parked in a portion of the vasculature during treatment. For example, the distal end of the sheath may be positioned in the right pulmonary artery, the left pulmonary artery, the pulmonary trunk, the right ventricle, the right atrium, the superior vena cava, or elsewhere as may be appropriate. If the sheath comprises a pressure sensor, for example, the position of the distal end of the sheath may be such that the pressure sensor is positioned in a desired body cavity (e.g., the right pulmonary artery, the left pulmonary artery, the pulmonary trunk, the right ventricle, the right atrium, the superior vena cava, or elsewhere as may be appropriate). Contrast, saline, heparin, and/or other fluids may be injected through the sheath, for example proximate to the expandable structure 320. Blood may be drawn through the sheath, for example to sample blood properties (e.g., SpO2) at the position of the distal end of the sheath. Preferably, motion of the sheath is not imparted to the expandable structure 320.

The sheath may comprise a radiopaque marker. The radiopaque marker may comprise, for example, an arcuate band.

The sheath may comprise a varying durometer. For example, the last 1-2 cm of the sheath can be very flexible to aid in recapturing the expandable structure 320.

The proximal portion may comprise an adapter comprising a plurality of ports, for example a first Y-adapter port and a second Y-adapter port. The first Y-adapter port may be in communication with a lumen configured to allow insertion of a guidewire through the system. The second Y-adapter port may comprise an electronics connector, which can be used to couple an electrode matrix of the system to a stimulator system.

The expandable structure 320 comprises a proximal portion 322 and a distal portion 324. The expandable structure 320 comprises a plurality of wires 326 and a plurality of electrode assemblies 330.

The wires 326 may include filaments, wires, ribbons, etc. having a circular cross-section, an arcuate non-circular cross-section (e.g., oval, ellipsoid, etc.), a rectangular cross-section (e.g., square), a trapezoidal cross-section, combinations thereof, and the like. In some examples, some wires 326 may have a cross-section configured to interact with a shape of an electrode assembly 330, a hub system, and/or other components. The wires 326 may have a diameter or lateral cross-section between about 0.002 inches (approx. 0.051 mm) and about 0.02 inches (approx. 0.51 mm) (e.g., about 0.002 inches (approx. 0.051 mm), about 0.004 inches (approx. 0.1 mm), about 0.006 inches (approx. 0.15 mm), about 0.008 inches (approx. 0.2 mm), about 0.01 inches (approx. 0.25 mm), about 0.012 inches (approx. 0.3 mm), about 0.015 inches (approx. 0.38 mm), about 0.02 inches (approx. 0.51 mm), ranges between such values, etc.). In some examples, some wires 326 may have a different diameter, for example configured to interact with a size of an electrode assembly 330, a hub system, and/or other components. In some examples, the wires ends that continue as spokes 328 may have a relatively lower diameter, for example to reduce an amount of material at the proximal portion 322.

The wires 326 may comprise, for example, nickel, titanium, chromium, cobalt, and alloys thereof including nickel titanium (e.g., nitinol), chromium cobalt, etc. The wires 326 may be heat treated to impart shape memory or superelasticity to the expandable structure 320. For example, the plurality of wires 326 may be heat treated so that the expandable structure 320 is in an expanded shape in the absence of external forces, and is collapsible to a compressed or delivery state (e.g., due to forces applied by the outer sheath). At least one of the wires 326 may comprise radiopaque material, for example drawn filled tubing with a radiopaque core and shape memory cladding, a radiopaque marker coupled to shape memory material, combinations thereof, and/or the like.

In some examples, the diameter of the expandable structure 320 in the expanded state is between about 15 mm and about 45 mm (e.g., about 15 mm, about 20 mm, about 22 mm, about 24 mm, about 26 mm, about 28 mm, about 35 mm, about 39 mm, about 43 mm, about 45 mm, ranges between such values, etc.). In some examples, the plurality of wires 326 are heat treated to be self-expanding such that the expandable structure 320 can self-expand from a compressed state for navigation to a target site to or towards the expanded state for treatment at a target site (e.g., a pulmonary artery (e.g., a right pulmonary artery, a left pulmonary artery, a pulmonary trunk), an inferior vena cava, a superior vena cava, an innominate vein, etc.). In certain such examples, the diameter of the expandable structure 320 in the expanded state may be oversized to most the intended vasculature of most subjects to ensure vessel wall apposition. The expanded state in a vessel may be less than a fully expanded state (e.g., in the absence of any radially inward forces due to the vessel wall). In some examples, the wires 326 may be non-self-expanding (e.g., balloon expanded, expanded like an umbrella with a wire, etc.). The expandable structure 320 can be used in various sizes of vessels (e.g., right pulmonary arteries of differing sizes), and the woven structure can adapt to the size of the vessel without compromising system performance. For example, the expandable structure 320 can appose vessel walls and push electrode assemblies against a vessel wall up to and including in a fully expanded state.

In some examples, the expandable structure 320 may be self-expanding, and may be further expanded (e.g., expanded with a wire, for example as described herein), which may provide an adjustable expandable structure 320 diameter usable for a range of vessels, vessel sizes, wall apposition forces, etc. Examples in which the expandable structure 320 does not appose the wall in the event of an error could be advantageous for safety, for example as described herein. Upon expansion of the expandable structure 320, the electrodes 336 of the electrode assemblies 330 may be selectively activated for testing nerve capture, calibration, and/or therapy, for example as described herein. In some examples, placing additional impedance sensing electrodes outside of the array can cause proximal movement of the braid to intersect the right pulmonary artery/left pulmonary artery bifurcation and move off the vessel wall into the blood stream.

When the plurality of wires 326 are woven, the weave pattern may be consistent along at least a portion of the expandable structure 320. In some examples, the weave pattern may be consistent from the distal end of the expandable structure 320 to the proximal end of the weaving (e.g., about where the spokes 328 are formed). Referring again to FIG. 3A, in some examples, the weave pattern is different between a first segment 340 and a second segment 342. For example, the first segment 340 may comprise a first braid angle 341 and the second segment 342 may comprise a second braid angle 343 different than the first braid angle 341. The second braid angle 343 may be greater than the first braid angle 341 (e.g., as shown in FIG. 3A), which can provide more outward radial force in an area comprising at least some of the electrodes 336. The second braid angle 343 may be less than the first braid angle 341, which can provide more outward radial force for more wall apposition in an area lacking electrodes 336. The expandable structure 320 may comprise more than two segments having changing properties. Other weave parameters may alternatively or additionally change between segments (e.g., braid pattern, picks per inch, porosity, density, etc.).

In some examples, deploying the expandable structure 320 comprises advancing the expandable structure 320 in a collapsed state distal to an expected target site. The expandable structure 320 may be expanded towards an expanded position. Combinations and parameters of the electrodes 336 may be tested. If the expandable structure is determined to be too distal, the expandable structure 320 may be proximally retracted without being collapsed. This retraction may use a bit of force, but may be less harmful to the vasculature and/or easier for the user than, for example, distally advancing the expandable structure 320 in an expanded state and/or resheathing the expandable structure 320, repositioning the expandable structure 320, and redeploying the expandable structure 320.

FIG. 3B is a proximal perspective view of a plurality of the example electrode assemblies 330-2 coupled to an example expandable structure 320. Coupling to other expandable structures, for example as described herein, is also possible. Sliding the electrode assembly 330-2 over proximal ends of filaments or struts can allow the electrode array to have various dimensional shapes (e.g., in an orthogonal view) or configurations such as rectangular, parallelogram (e.g., as shown in FIG. 3B), staggered, etc. while using the same or substantially the same electrode assembly 330-2 for each electrode assembly 330-2 of the electrode array. In a parallelogram arrangement, the distal end of the lumen is positioned more distally for each circumferentially adjacent electrode assembly 330-2. In some implementations, the filaments may be truncated at a position In which the proximal end of the filament Is in the tube of the respective electrode assembly 330-2. In some implementations, a length of the tube proximal to the upper insulator may be longer for electrode assemblies 330-2 positioned more distally on the expandable structure 320. A parallelogram may be easier to capture than an electrode array having a different shape. For example, in a rectangular electrode array, the proximal-most set electrode assembly may bunch when being recaptured in an outer sheath. Staggering the electrode assemblies in a parallelogram may allow more movement for each electrode assembly to find a lower energy state during recapture. The parallelogram shape or arrangement may be considered based on precise spacing between similar or identical components (e.g., a proximal end (e.g., without a proximal tab, a proximal-most electrode) of each electrode assembly is distal to the proximal end of a circumferentially adjacent electrode assembly and wherein a distal end (e.g., without a distal tab, a distal-most electrode) of each electrode assembly is distal to the distal end of a circumferentially adjacent electrode assembly). The parallelogram shape or arrangement may be considered based on an overall shape, for example drawing a rough outline around the electrode array and/or the plurality of electrodes as a whole. Other electrode arrays and expandable structures described herein may also take any suitable shape.

The right pulmonary artery is usually angled anterior to posterior, meaning that it heads posterior as it goes to the right of the subject. Anterior electrodes of an electrode array in a rectangular configuration deployed in the right pulmonary artery to be more to the right of the subject, which may be further away from a target zone. When an electrode array having a parallelogram configuration is deployed in a vessel such as right pulmonary artery that turns proximate a target site, the parallelogram can act and/or appear as more vertical, better filling the target zone. The electrode array shaped as a parallelogram may comprise a plurality of electrode assemblies each having a linear array of electrodes.

The distal-most electrode 336-1 in the expanded state is also the distal-most electrode in the compressed or collapsed state and in a partially expanded state. The distal-most electrode 336-1 can be used to longitudinally and/or rotationally align the expandable structure 320 and/or the electrode assemblies 330. In some examples, the distal-most electrode 336-1 is positioned superior in the vessel (e.g., the right pulmonary artery). The distal-most electrode 336-1 can be used as a radiopaque marker (e.g., in addition to or instead of the radiopaque marker 325). Knowing the position of the distal-most electrode 336-1 and its orientation 0° from the superior-most electrode assembly 330-1 can provide a user with information that the other electrode assemblies 330 are anterior to the superior-most electrode assembly 330-1 when the distal-most electrode 336-1 is in a superior position, which can provide information that all of the electrode assemblies 330 are in a target zone (e.g., superior to anterior). In some examples, the expandable structure 320 may be rotated after initial alignment such that the electrode assemblies are rotated. For example, after superior alignment using the distal-most electrode 336-1, the expandable structure 320 may be rotated between about 5° and about 850 (e.g., about 5°, about 15°, about 25°, about 35°, about 45°, about 55°, about 65°, about 75°, about 85°, and ranges between such angles). The rotation may be clockwise or counterclockwise. This rotation can aid in providing an improved or optimal position of the electrode assemblies 330 in the target zone. In some examples, the distal-most electrode 336-1 can be longitudinally aligned with the trachea carina, which is the right-left bifurcation of the primary bronchi, or the left margin of the trachea (e.g., as discussed with respect to FIG. 2F). If repositioning of the expandable structure 320 and/or the electrode assemblies 330 is desired, the user can use the distal-most electrode 336-1 as a datum (e.g., saving a ghost fluoroscopy view) and adjust to a second location.

FIG. 3Ci is top, side, and proximal perspective view of another example electrode assembly 360. FIG. 3Cii is back, side, and proximal perspective view of the example electrode assembly 360. The electrode assembly 360 may share features with other electrode assemblies described herein (e.g., the electrode assemblies 330) and may have some different, additional, and/or fewer features. For example, the electrode assembly 360 includes a lower insulator 361, an upper insulator 363, and electrodes 366.

The lower insulator 361 can comprise a channel. The upper insulator 363 can also comprise a channel. Together, the channels of the lower insulator 361 and the upper insulator 363 form a lumen 364. Electrical conductors can be coupled to respective electrodes 366 and positioned in the lumen 364. The lumen 364 may have an open proximal end (e.g., as shown in FIGS. 3Ci and 3Cii) and a closed distal end. Such a configuration may ease manufacturing, for example by only sealing one end after coupling electrical conductor wires, attaching to an expandable structure, etc. The lumen 364 may have an open proximal end (e.g., as shown in FIGS. 3Ci and 3Cii) and an open distal end. Such a configuration may ease manufacturing, for example by allowing the lower insulator 361 and the upper insulator to be used in either longitudinal orientation.

In some implementations, the electrode assembly 360 comprises a distal tab. The distal tab can help to inhibit or prevent the distal end of the electrode assembly 360 from protruding through open cell areas of an expandable structure. In some implementations, the distal tab has a length that is at least about 10% larger, at least about 25% larger, at least about 50% larger, at least about 75% larger, at least about 100% larger, or even larger, than a longitudinal length of a cell in a fully expanded position. The distal tab should not protrude distally beyond the expandable structure. The distal tab may protrude less than about five, less than about four, less than about three, less than about two, etc. cell lengths, and greater than one cell length. The electrode assembly 360 and/or a portion or portions of the electrode assembly 360 maybe annealed with an upward or outward curve. Such annealing could bias a distal tab away from an expandable structure, reducing the risk of the electrode assembly 360 protruding through the expandable structure.

FIG. 3Ciii is bottom plan view of an example upper insulator 363 and example electrodes 366 of the example electrode assembly 360. In some implementations, the upper insulator 363 could omit the channel 365 and/or the recesses 367, leaving apertures for the upper surfaces of the electrodes 366. In certain such implementations, the lower insulator 361 can comprise the lumen 364 and/or the recesses 367. If the upper insulator 363 is too thin, manufacturing may become difficult, for example because tears can occur at thin points (e.g., proximate to electrode apertures). In some implementations, the upper insulator has a thickness between about 0.006 inches (approx. 0.15 mm mm) and about 0.012 inches (approx. 0.3 mm) (e.g., about 0.006 inches (approx. 0.15 mm), about 0.007 inches (approx. 0.18 mm), about 0.008 inches (approx. 0.2 mm), about 0.009 inches (approx. 0.23 mm), about 0.01 inches (approx. 0.25 mm), about 0.011 inches (approx. 0.28 mm), about 0.012 inches (approx. 0.3 mm), ranges between such values, etc.). Different materials may have different manufacturable thickness ranges. As discussed above, the upper insulator 363 includes a channel 365 that at least partially defines the lumen 364. The upper insulator 363 includes recesses 367 configured to receive the electrodes 366. The upper insulator 363 including some features and the lower insulator 361 including some features can increase the thickness of the upper insulator 363, reducing the risk of tears or other defects during manufacturing.

FIG. 3Civ is side view of a plurality of the example electrode assemblies 360 coupled to an expandable structure 320. FIG. 3Cii shows that the lower insulator 361 comprises a first aperture 362p and a second aperture 362d. The second aperture 362d is distal to the first aperture 362p. When the electrode assembly 360 is coupled to an expandable structure (e.g., the expandable structure 320), filaments of the expandable structure (e.g., the wires 326c, 326t) can enter the lumen 364 through one of the apertures 362p, 362d. The filaments can extend proximally and exit the lumen 364 with the conductors.

FIG. 3D illustrates an example expandable device 370 for positioning in a cavity of a subject. The device 370 comprises a plurality of filaments woven to form a mesh structure 380, for example sharing features with the expandable structure 320. The structure 380 may be longer, for example to support a first electrode array 372 and a second electrode array 374. The device 370 may comprise one array, two arrays, three arrays, four arrays, or more. Each electrode array may comprise one electrode assembly or a plurality of electrode assemblies (e.g., two electrode assemblies, three electrode assemblies, four electrode assemblies, five electrode assemblies, six electrode assemblies, six electrode assemblies, six electrode assemblies, six electrode assemblies, six electrode assemblies, six electrode assemblies, six electrode assemblies, and ranges between such values). For example, the electrode arrays 372, 374 shown in FIG. 3D each comprise four electrode assemblies. In some embodiments, the electrode assemblies may be uniformly circumferentially spaced. For example, over 1200 of circumferential coverage, four electrode assemblies may each be spaced from a circumferentially adjacent electrode assembly by about 40°. The electrode assemblies may cover part of a circumference or be spaced around an entire circumference. Each electrode assembly may comprise one electrode or a plurality of electrodes (e.g., two electrodes, three electrodes, four electrodes, five electrodes, six electrodes, eight electrodes, ten electrodes, twelve electrodes, fourteen electrodes, sixteen, and ranges between such values). For example, the electrode assemblies in FIG. 3D each comprise four electrodes. Different electrode arrays can comprise different numbers of electrode assemblies, and different electrode assemblies can comprise different numbers of electrodes.

The first electrode array 372 may share features with the electrode array described with respect to FIG. 61Eix, for example. The first electrode array 372 may be configured to be positioned in a right pulmonary artery. The first electrode array 372 can apply stimulation to nerves around the right pulmonary artery to increase autonomic function or balance (e.g., cardiac contractility and/or relaxation), for example as described herein. This first electrode array 372 can be the array used to block the sympathetic release of norepinephrine via blocking efferent signals of the post-ganglionic cardiac nerve.

The second electrode array 374 is distal to the first electrode array 372. The second electrode array 374 may be circumferentially aligned with the first electrode array 372 (e.g., as shown in FIG. 3D). The second electrode array 374 may be circumferentially offset from the first electrode array 372. The second electrode array 374 may be longitudinally spaced from the first electrode array 372 (e.g., as shown in FIG. 3D). The second electrode array 374 may share features with the first electrode array 372, may be a modification thereof (e.g., comprising fewer electrodes), or may be different. For example, the electrode assemblies of the second electrode array 374 may be differently coupled to the structure 380. The second electrode array 374 is configured to be positioned in the right pulmonary artery distal to the first electrode array 372. In this position, the second electrode array 374 can stimulate aortic baroreceptors to achieve sympathetic inhibition or reduction upon detection of autonomic imbalance (e.g., sympathetic overdrive). For example, the device 370 or a separate device may comprise a sensor 376 configured to detect autonomic imbalance (e.g., sympathetic overdrive), such as, for example, an EKG analysis circuit detecting decreased heart rate variability, a pressure monitor showing pressure that is too high, too low, and/or too variable, skeletal or muscle sympathetic nerve activity (MNSA) change showing possible norepinephrine spillover, a biomarker contained within a bodily fluid or tissue, etc.). The waveforms and other properties of the stimulation by the second electrode array 374 may be different than properties of the stimulation by the first electrode array 372. In some implementations, the second electrode array 374 may effect radiofrequency ablation.

An example method of using the device 370 is a subject that presents with heart failure. The device 370 is positioned with the first electrode array 372 in the right pulmonary artery to modulate nerves of the autonomic system (e.g., to modulate cardiac contractility or autonomic function), for example as discussed herein. The subject may experience autonomic imbalance (e.g., chronically compensatory overactive sympathetic drive), which over time can prematurely wear out the heart. Beta blockers could be used to inhibit receptors from receiving chemicals that can cause hyperactivity and/or calcium blockers could be used to inhibit the response to received norepinephrine. These chemical treatments could cause undesirable side effects. Additionally or alternatively, the second electrode array 374 of the device 370 can be used to shut down the neuron that would release norepinephrine, which would not cause systemic beta blocker side effects. The second electrode array 374 could be operated alternatingly with the first electrode array 372. For example, the stimulation of the second electrode array 374 and the first electrode array 372 can be time multiplexed. For example, stimulation of the first electrode array 372 can increase sympathetic tone to augment norepinephrine release to augment contractility, and stimulation of the second electrode array 374 can decrease sympathetic tone to inhibit norepinephrine release to mimic beta-blockade at different times. The second electrode array 374 could be operated at least partially simultaneously with the first electrode array 372. For example, stimulating with both the second electrode array 374 and the first electrode array 372 (e.g., at least partially simultaneously), can increase contractility substantially without affecting heart rate, which can, for example, improve stroke volume and/or manage increases in myocardial oxygen consumption. In some implementations, the second electrode array 374 could be on a separate device.

In some embodiments, the second electrode array 374 could be used to stimulate the parasympathetic nervous system. Stimulating the parasympathetic nervous system can result in release of ACH. Release of ACH can block norepinephrine release. Release of ACH may also or alternatively cause side effects such as reduced heart rate, increased heart rate variability, direct antiarrhythmic effects, anti-inflammatory effects, change in nitric oxide expression, change in cytokine expression, inhibition of the renin-angiotensin system, and/or improved baroreflex sensitivity, one, some or all of which may actually be desired.

The device 370 optionally comprises a pressure sensor 376. The pressure sensor 376 may be, for example, a MEMS pressure sensor. Other types of pressure sensors or other sensors are also possible. If the device 370 is chronically implantable, the device 370 may comprise implantable electronics 378, which may include, for example, a power source (e.g., rechargeable battery), stimulation circuitry (e.g., configured to provide signals to the first electrode array 372 and/or the second electrode array 374), wireless communication circuitry, and/or other circuitry. For example, in some implementations, the pressure sensor 376 and/or a different pressure sensor or other type of sensor (e.g., indicative of cardiac output or performance such as an O2 sensor) may be regularly (e.g., daily) interrogated to determine if therapy is desirable. The electronics 378 and/or a case containing the electronics 378 may be flexible, for example for bending during navigation and/or in response to vessel changes. For subjects already having a pacemaker or defibrillator, some of the electronics from such a device may be connected to the device 370 by tapping into a lead, placing a new lead, etc.

The proximal ends of the filaments of the structure 380 may form spokes and be joined at a hub, for example as described herein. Such a device 370 may be used as a temporary (e.g., less than about one week) intravascular implant. The proximal ends of the filaments may be coupled, form bends, form loops, be braided back, etc. and releasable from a catheter. Such a device 370 may be chronically implantable. The proximal and/or distal ends of the device 370, and/or positions therebetween, may comprise anchors to aid in position stabilization.

FIG. 3E is an example schematic of 16 electrodes numbered from 1 in the upper right to 16 in the lower left. Other numbers and configurations of electrodes are also possible. Combinations of electrodes can be tested for undesired effects. At least one of the parameters for the testing may be at non-stimulation levels, for example a frequency of about 2 Hz. Electrodes involved in combinations that exhibit undesired effects, for example affecting heart rate, causing changes to EKG such as amplitude(s), extra P waves, etc., can be omitted from further testing. For example, testing may comprise the following electrode combinations: 15, 5 9, 9 13, 2 6, 6 10, 10 14, 3 7, 7 11, 11 15, 4 8, 8 12, and 12 16. As described herein, the nerves believed to affect autonomic function or balance (e.g., contractility and/or relaxation) should run parallel to the electrode array such that these combinations that are transverse to the longitudinal axes of the electrode assemblies should be most likely to capture the nerve(s). For other arrays, targeting other nerves, etc. different pairs of electrodes can be used. Additional pairs of electrodes can also be tested, including pairs parallel to the longitudinal axes if the electrode assemblies. Electrodes from combinations that affect heart rate, indicative of undesired effects can be excluded while electrodes from combinations that do not affect heart rate may continue to be tested. For the sake of the example, assume that combinations 1 5, 5 9, 2 6, and 6 10 showed undesired effects. Thus, electrodes 1, 2, 5, 6, 9, and 10 may be excluded from all future testing. If the method is restarted (e.g., due to movement of the electrode array), the electrodes 1, 2, 5, 6, 9, and 10 may be reincluded in the testing. Exclusion of electrodes that show undesired effects can avoid arrhythmias and/or save time in later steps.

In FIG. 3Fi, a first signal is sent to the electrode array to cause electrode 1 (referring again to the numbering system of FIG. 3E) to be a cathode, electrode 2 longitudinally adjacent to electrode 1 on the same electrode assembly to have no charge, and the remainder of the electrodes to be an anode. In FIG. 3Fii, a second signal is sent to the electrode array to cause electrode 2 to be a cathode, electrodes 1 and 3 longitudinally adjacent to electrode 2 on the same electrode assembly to have no charge, and the remainder of the electrodes to be an anode. In FIG. 3Fiii, a third signal is sent to the electrode array to cause electrode 3 to be a cathode, electrodes 2 and 4 longitudinally adjacent to electrode 2 on the same electrode assembly to have no charge, and the remainder of the electrodes to be an anode. In FIG. 3Fiv, a fourth signal is sent to the electrode array to cause electrode 4 to be a cathode, electrode 3 longitudinally adjacent to electrode 4 on the same electrode assembly to have no charge, and the remainder of the electrodes to be an anode. In FIG. 3Fv, a fifth signal is sent to the electrode array to cause electrode 5 to be a cathode, electrode 6 longitudinally adjacent to electrode 5 on the same electrode assembly to have no charge, and the remainder of the electrodes to be an anode. In FIG. 3Fvi, a sixth signal is sent to the electrode array to cause electrode 6 to be a cathode, electrodes 5 and 7 longitudinally adjacent to electrode 6 on the same electrode assembly to have no charge, and the remainder of the electrodes to be an anode. In FIG. 3Fvii, a seventh signal is sent to the electrode array to cause electrode 7 to be a cathode, electrodes 6 and 8 longitudinally adjacent to electrode 7 on the same electrode assembly to have no charge, and the remainder of the electrodes to be an anode. In FIG. 3Fviii, an eighth signal is sent to the electrode array to cause electrode 8 to be a cathode, electrode 7 longitudinally adjacent to electrode 8 on the same electrode assembly to have no charge, and the remainder of the electrodes to be an anode. The method may optionally continue in the same pattern with ninth, tenth, eleventh, twelfth, thirteenth, fourteenth, fifteenth, and sixteenth signals for each of electrodes 9, 10, 11, 12, 13, 14, 15, and 16, respectively. Electrode(s) adjacent to the cathode on the same electrode assembly are turned off during this unipolar testing. A system may be configured to perform the tests, for example automatically or semi-automatically (e.g., after input from a user (e.g., practitioner or clinical professional) to move to the next electrode, set of electrodes, test, etc.).

The unipolar test may be performed one or more times. For example, a first test may be at 20 Hz, 4 ms, and 8 mA. A second test may be at 20 Hz, 4 ms, and 16 mA (double current amplitude) or 4 mA (half current amplitude). Each test may be, for example, 10 s, 15 s, 20 s, 30 s, 45 s, 1 min, and ranges including such values. Parameters (e.g., frequency, pulse width, etc.) other than or in addition to current amplitude may be varied. Preferably, only one parameter is varied between tests. The change in current may elicit different responses, for example between sympathetic and parasympathetic. There may be a pause between the first test and the second test for each electrode before moving on to test the next electrode, some or all of the electrodes can be tested with the first test followed by the second test, some or all of the electrodes on one electrode assembly can be tested with the first test following by testing some or all of the electrodes on one electrode assembly with the second test and then repeated for the other electrode assemblies, some or all of the distal electrodes can be tested with the first test following by testing all of the distal electrodes with the second test and then moving proximal, some or all of the proximal electrodes can be tested with the first test following by testing some or all of the proximal electrodes with the second test and then moving distal, using a binary search tree, and other combinations and permutations are also possible without departing from the method.

During the test and/or during parameter setting, during neuromodulation, and/or at other appropriate times, heart rate and/or contractility and/or relaxation response may be monitored. For example, a reduction in heart rate may be indicative that the test evoked a parasympathetic response. For another example, an increase in contractility may be indicative that the test evoked a sympathetic response. Other parameters may also be monitored, for example, but not limited to, arterial blood pressure (ABP) and its derivatives (e.g., mean arterial pressure (MAP), systole blood pressure (SBP), diastole blood pressure (DBP), pulse pressure (PP; SBP-DPB), ABP dP/dtₘₐₓ, and/or ABP dP/dtₘᵢₙ, pulmonary artery pressure and/or its derivatives (e.g., mean (PAPm), systolic (PAPs), diastolic (PAPd), pulmonary artery pulsatility index (PAPi)), right ventricular pressure and/or its derivatives (e.g., mean (RVPm), systolic (RVPs), diastolic (RVPd), pulse pressure (PP; RVPs-RVPd), RV dP/dtₘₐₓ, and/or RV dP/dtₘᵢₙ)), measures of changing blood volume in the peripheral circulation (e.g., plethysmograph, pulse oximetry), measures of changing blood chemistry (e.g., blood oxygenation in the venous and/or arterial compartments), measures of changing blood volume in the thorax and/or abdomen (e.g., trans thoracic impedance, conductance admittance), measures of changing blood volume in a chamber or vessel (e.g., changing impedance, changing conductance, admittance in blood vessel, admittance in cardiac chamber), intravascular ultrasound changes (e.g., doppler shifts in a vessel of interest), transthoracic and/or transesophageal ultrasound changes (e.g., changes in cardiac dimensions, changes in cardiac volumes, changes in flow through cardiac valves (e.g., as measured by doppler), changes in cardiac motion (e.g., as measured by tissue doppler)), rhythm changes, and/or combinations thereof. If all electrodes fail to show any effect, additional tests can be performed under different parameters (e.g., at 25 mA).

The method may be performed in addition to or alternative to the methods described herein. For example, if electrodes on adjacent assemblies are tested first (e.g., 1-5, 2-6, etc.) and show undesired effects (e.g., atrial capture (e.g., as indicated by at least one effect on the atrium such as, for example, change in atrial output, change in P wave activity, change in QRS morphology, other change in EKG, stimulation to atrial electrogram interval at the proximal coronary sinus, stimulation to atrial electrogram interval at the high right atrium, control of an atrium for one or more beats after a period of independent beating, as in incomplete AV block or in junctional or ventricular ectopic beats or tachycardias by a retrograde impulse), coughing, patient intolerance), then the expandable structure may be repositioned, one or more electrode assemblies showing the undesired effects may be discarded, and/or amplitude may be reduced. For example, if 9-13 shows undesired effects, then the electrode assemblies including electrodes 9-16 may be discarded such that the unipolar testing is only performed on electrodes 1 to 8 as illustrated in FIGS. 3Fi-3Fviii. For another example, if 9-13 shows undesired effects but 5-9 does not, then the electrode assembly including electrodes 13-16 may be discarded such that the unipolar testing is only performed on electrodes 1 to 12 (FIGS. 3Fi-3Fviii and electrodes 9 to 12). Other procedures described herein and otherwise may also be performed at a plurality of amplitudes to identify combinations that can evoke sympathetic and/or parasympathetic responses.

Upon identification of electrodes that are able to evoke sympathetic and parasympathetic responses, the electrodes that evoke sympathetic responses can be used to stimulate autonomic function or balance (e.g., cardiac contractility and/or relaxation), for example as described herein.

### Therapy Titration

FIG. 4 is a flowchart of an example of an optimization method 400 for stimulation therapy. Stimulation therapy, such as cardiac pulmonary nerve stimulation (CPNS) therapy, can be used to force a restart of the local control circuit of the cardiac autonomic nervous system (CANS), or more specifically the intracardiac nervous system (ICNS). Different vectors of stimulation, for example stimulation from electrodes in different positions on the cardiac plexus, can cause the ICNS to shift to different states. The goal of this optimization method 400 can be to determine a vector of stimulation that shifts the ICNS to an optimal state. In some embodiments, the optimization method 400 can be used for closed-loop therapy delivery, where the parameters of the stimulation change automatically based on the measured physiological parameters. This optimization method 400 can have applications outside of CPNS. For example, the optimization method 400 can be applied to other forms of neurostimulation, stimulation of other vessels, stimulation by a chronic device, use of a drug pump, use of piezoelectric crystal (for example with an ultrasonic source), and use of balloon therapy (as described in FIGS. 3A and 3B). Before testing, the method can include using fluoroscopy to determine the anatomical position of the CPNS catheter and determining the vectors or electrode pairs to use for stimulation. The electrodes used for stimulation can be similar to those described in FIGS. 3A, 3B, 3Ci-iv, 3D, 3E, and 3Fi-viii.

The optimization method 400 can be conducted by a stimulation system, for example as described in FIG. 13. The stimulation system can include a device for electrical stimulation, such as the devices of FIGS. 3A, 3B, 3Ci-iv, 3D, 3E, and 3Fi-viii. The stimulation system can include sensors for measurement of physiological parameters, such as an electrocardiogram sensor or electrode, a pulse oximeter, a contractility sensor, a pressure sensor, and combinations thereof. The stimulation system can include a controller to initiate, stop, or modify the electrical stimulation, either automatically or by a user. The stimulation system can include a processor to calculate changes in physiological parameters and determine therapy efficacy or goodness, for example as described in FIGS. 7A-B and 8.

At block 402, the method can include measuring baseline physiological parameters of the patient or subject. The physiological parameters can include contractility, lusitropy, and/or chronotropy. In some embodiments, the physiological parameters can also include preload, systolic function, and/or other parameters. The baseline physiological parameters can be the parameters of the patient or subject before stimulation is applied. Baseline physiological parameters can be measured using one or more sensors, such as an electrocardiogram sensor or electrode, a pulse oximeter, a contractility sensor, a pressure sensor, and combinations thereof.

At block 404, the method can include applying nerve stimulation to the patient or subject. Nerve stimulation can include CPNS therapy, for example, on the cardiac plexus at the level of the right pulmonary artery. However, nerve stimulation can include other types of nerve stimulation or other targets. Nerve stimulation can be applied using the systems and devices described herein, for example in FIGS. 3A and 3B. The stimulator can deliver stimulation pulses to any individual electrode, electrode pair, or multiple electrodes in the catheter with a frequency of 2 Hz to 40 Hz (e.g., 2 Hz to 20 Hz, 5 Hz to 25 Hz, 10 Hz to 30 Hz, 20 Hz to 40 Hz, overlapping ranges thereof, or any value within the recited ranges), with a pulse width of 100 μs to 4 ms (e.g. 100 μs to 2 ms, 1.5 ms to 3 ms, 2 ms to 4 ms, 100 μs to 3 ms, overlapping ranges thereof, or any value within the recited ranges), for a duration of 20 seconds to 120 seconds (e.g., 20 seconds to 60 seconds, 40 seconds to 80 seconds, 60 seconds to 120 seconds, overlapping ranges thereof, or any value within the recited ranges), at intervals of 5 minutes to 20 minutes (e.g., 5 minutes to 15 minutes, 10 minutes to 20 minutes, overlapping ranges thereof, or any value within the recited ranges), and with an amplitude or intensity of 0.1 mA to 20 mA (e.g., 0.1 mA to 1 mA, 0.5 mA to 5 mA, 1 mA to 10 mA, 5 mA to 15 mA, 10 mA to 20 mA, overlapping ranges thereof, or any value within the recited ranges). In some embodiments, the stimulator can deliver stimulation pulses with a frequency of 1 to 60 Hz (e.g., 1 Hz to 20 Hz, 10 Hz to 25 Hz, 15 Hz to 30 Hz, 10 Hz to 40 Hz, 20 Hz to 50 Hz, 25 Hz to 60 Hz, 40 Hz to 60 Hz, overlapping ranges thereof, or any value within the recited ranges), with a pulse width of 100 μs to 10 ms (e.g., 100 μs to 4 ms, 1.5 ms to 5 ms, 2 ms to 6 ms, 3 ms to 8 ms, 4 ms to 10 ms, 6 ms to 8 ms, 7 ms to 10 ms, overlapping ranges thereof, or any value within the recited ranges), for a duration of 10 seconds to 240 seconds (e.g., 10 seconds to 60 seconds, 30 seconds to 90 seconds, 45 seconds to 135 seconds, 60 seconds to 120 seconds, 100 seconds to 200 seconds, 120 seconds to 240 seconds, overlapping ranges thereof, or any value within the recited ranges), at intervals of 1 minute to 40 minutes (e.g., 1 minute to 10 minutes, 5 minutes to 15 minutes, 10 minutes to 30 minutes, 15 minutes to 35 minutes, 20 minutes to 40 minutes, overlapping ranges thereof, or any value within the recited ranges), and with an amplitude of 0.1 mA to 40 mA (e.g., 0.1 mA to 1 mA, 0.5 mA to 5 mA, 2 mA to 10 mA, 5 mA to 20 mA, 5 mA to 10 mA, 10 mA to 30 mA, 15 mA to 40 mA, overlapping ranges thereof, or any value within the recited ranges). The patient can be stimulated with trains of pulses for 20 to 120 seconds (e.g., 20 seconds to 60 seconds, 40 seconds to 90 seconds, 45 seconds to 120 seconds, 90 seconds to 120 seconds, 60 seconds to 120 seconds, overlapping ranges thereof, or any value within the recited ranges) and then stimulation can be stopped for 5 minutes to 20 minutes (e.g., 5 minutes to 15 minutes, 10 minutes to 20 minutes, overlapping ranges thereof, or any value within the recited ranges). In some embodiments, the patient can be stimulated with trains of pulses for 10 seconds to 240 seconds (e.g., 10 seconds to 60 seconds, 30 seconds to 90 seconds, 60 seconds to 180 seconds, 60 seconds to 120 seconds, 90 seconds to 180 seconds, 90 seconds to 240 seconds, 120 seconds to 240 seconds, overlapping ranges thereof, or any value within the recited ranges) and then stimulation can be stopped for 1 minute to 40 minutes (e.g., 1 minute to 10 minutes, 5 minutes to 15 minutes, 10 minutes to 30 minutes, 15 minutes to 40 minutes, 20 minutes to 40 minutes, overlapping ranges thereof, or any value within the recited ranges). This sequence can be repeated for 2 hours and then all stimulation can be turned off for 2 hours. In some embodiments, this sequence can be repeated for 1 to 4 hours (e.g., 1 hour to 3 hours, 2 hours to 4 hours, 1.5 hours to 3 hours, 1 hour, 1.5 hours, 2 hours, 2.5 hours, 3 hours, 3.5 hours, 4 hours) and then all stimulation can be turned off for 1 to 4 hours (e.g., 1 hour to 3 hours, 2 hours to 4 hours, 1.5 hours to 3 hours, 1 hour, 1.5 hours, 2 hours, 2.5 hours, 3 hours, 3.5 hours, 4 hours). This cycle can be repeated for 48 hours or other desired duration. In some embodiments, this cycle can be repeated for 12 hours to 72 hours (e.g., 12 hours to 36 hours, 24 hours to 36 hours, 36 hours to 48 hours, 48 hours to 72 hours, overlapping ranges thereof, or any value within the recited ranges). This can include a weaning cycle of 2 hours where stimulation is gradually reduced. In some embodiments, the weaning cycle can be 1 hour to 5 hours (e.g., 1 hour to 3 hours, 2 hours to 4 hours, 1.5 hours to 3 hours, 2 hours to 5 hours, 1 hour, 1.5 hours, 2 hours, 2.5 hours, 3 hours, 3.5 hours, 4 hours, 4.5 hours, 5 hours), or another desired duration.

Stimulating, or adding noise to the ICNS, can improve inotropy at the ICNS level. Another mode for improving the ICNS, CANS, or autonomic nervous system can include mechanically stimulating the pulmonary artery baroreceptors using mechanical force to trigger a response to that stimulation. This mechanical stimulation can be achieved, for example, by deploying a balloon that stretches the baroreceptors, and repeatedly inflating the balloon to generate the stimulation at intervals, for example 10 minutes or other desired duration, and in pulses, for example 1 minute each, or other desired duration. This balloon can preserve blood flow through the artery during its periods of inflation. Another mode for improving the ICNS, CANS, or autonomic nervous system can include using a drug pump to infuse inotropes that affect the ICNS differentially for brief periods, for example 1 minute with 10 minutes in between, for 2 hours and then rest for 2 hours. Drug dosage can be weaned, for example for the last 2 hours and treatment can take place for 48 hours. Other durations may be used as desired and/or required.

Another mode for improving the ICNS, CANS, or autonomic nervous system can include implanting piezoelectric crystals in the region of the cardiac plexus that can either be left there, reabsorbed or explanted at a later time. Stimulating those crystals with an ultrasound pulse can create the cardiac plexus stimulation. Other drug, electrical, mechanical or ultrasound forms of stimulation of the ICNS in brief periods of time can be used (for example for 1 minute on 10 minutes off, and 2 hours on 2 hours off). These forms of stimulation can also include weaning at the end. The systems described herein can be triggered by a sensor that detects decompensation or edema. For example, a decrease in transthoracic impedance or leg plethysmography impedance can trigger the stimulation. The systems described herein can use a titration algorithm to determine the exact dosage to use for each potential means of applying the therapy, for example drug dosage, mechanical force/pressure in the balloon, and/or electrical amplitude.

At block 406, the method can include measuring the change in physiological parameters of the patient or subject. The physiological parameters can include contractility, lusitropy, chronotropy, and/or dromotropy. In some embodiments, the physiological parameters can also include preload, systolic function, and/or other parameters. The change in physiological parameters can be a comparison of the parameters of the patient or subject after stimulation and the baseline physiological parameters. The hemodynamic response of the patient or subject can be determined using one or more hemodynamic and electrocardiographic sensors, for example. The method can account for sensations experienced by the patient or subject. In some embodiments, if the patient or subject experiences a sensation, a significant sensation, or a painful sensation, the stimulation can be reduced or stopped. Physiological parameters can be measured using one or more sensors, such as an electrocardiogram sensor or electrode, a pulse oximeter, a contractility sensor, a pressure sensor, and combinations thereof. Dromotropy, or the change in conduction velocity, can be measured via intracardiac electrograms and/or electrocardiograms timing changes. Dromotropy can provide insight into contractility changes without direct left ventricular pressure measurements.

At block 408, the method can include determining therapy efficacy. The efficacy of the therapy can be determined based on the change in physiological parameters, hemodynamic response, and/or sensations of the patient or subject. In some embodiments, the efficacy of therapy can be determined using the goodness indicators of FIGS. 7A, 7B, and 8 or a similar multi-factor algorithm or protocol. In some embodiments, an increase in contractility, a decrease in preload, an increase in lusitropy, an increase in systolic blood pressure, and a decrease in chronotropy can indicate positive therapy efficacy or hemodynamic response, either in isolation or in combination. In some embodiments, a decrease in contractility, an increase in preload, a decrease in lusitropy, a decrease in systolic blood pressure, and an increase in chronotropy can indicate negative therapy efficacy or hemodynamic response, either in isolation or in combination.

In some embodiments, the stimulation system can include monitoring the patient's hemodynamics using right ventricular pressure, pulmonary artery pressure, electrocardiogram, or direct electrogram from a CPNS device (e.g., basket, woven structure or expandable structure) using non stimulating electrodes chosen to maximize signal to noise ratio, and extracting breathing from these signals. The stimulation system can include ensuring that the patient or subject's oxygen consumption does not exceed availability by monitoring for some or all of the following: arrhythmias, ectopic activity, regularity of hearth rhythm, mechanical alternans, and/or hemodynamics.

At block 410, the method can include adjusting therapy delivery based on the therapy efficacy determined by the goodness indicator or other multi-factor algorithm or protocol. Adjusting therapy delivery can include changing the electrode position, electrode configuration, and/or the stimulation intensity. Stimulation intensity can include, for example, stimulation amplitude, frequency, pulse width, and duty cycle. In other examples, adjusting the property of the one or more electrical pulses can include changing which of an electrode or plurality of electrodes on a neurostimulation catheter (such as catheters described herein) is used to deliver the one or more electrical pulses. In another example, adjusting the property of the one or more electrical pulses can include moving the catheter to reposition one or more electrodes of the catheter in the pulmonary artery of the heart. In yet another example, adjusting the property of the one or more electrical pulses can include changing at least one of an electrode polarity, a pulsing mode, a pulse width, an amplitude, a frequency, a phase, a voltage, a current, a duration, an inter-pulse interval, a duty cycle, a dwell time, a sequence, a wavelength, and/or a waveform of the one or more electrical pulses.

The optimization method 400 can be conducted at a variety of stimulation vectors, stimulation intensities, and with other varied stimulation parameters. At block 410, therapy delivery can be adjusted to the parameters with a positive, or the most positive (e.g., beneficial or clinically efficacious), therapy efficacy. In some embodiments, the stimulation system can present a user (e.g., clinical professional) with the optimal parameters (e.g., on a display), and the user can manually adjust the stimulation parameters.

The stimulation system can stimulate the ICNS to force it to reassess locally and at the level of the autonomic nervous system and CANS (for cardiac) or autonomic nervous system (for central) the maladaptive state it is in. The maladaptive state can include, for example, acute decompensated heart failure, which can contribute to a patient's decompensated state. What worked before may now be deleterious for the patient. The systems herein can allow the patient or subject to reach a better state, a normal parameter for acute decompensated heart failure, or a virtuous state.

FIG. 5 illustrates an example of staged atrial capture (AC) tests at different vectors. The tests can be conducted by a system similar to the system of FIG. 4. The method can include gradually increasing the amplitude of each vector. Each vector test can include 3 tests. In some embodiments, each vector test can include 1-10 tests (e.g., 1 to 4 tests, 2 to 5 tests, 3 to 8 tests, 4 to 10 tests, overlapping ranges thereof, or any value within the recited ranges). For each of the tests, an initial stimulation at a first frequency, for example 2 Hz, for 5 seconds can be conducted, followed by a break of less than 5 seconds, then followed by a stimulation at a second, higher frequency, for example 20 Hz, for 30 seconds. In some embodiments, each test can include an initial stimulation at a frequency 1 Hz to 10 Hz for 1 second to 10 seconds, followed by a break of 1 second to 10 seconds, then followed by a stimulation at a frequency of 10 Hz to 40 Hz for 10 seconds to 120 seconds. Stimulation can be conducted, for example, using devices similar to those described in FIGS. 3A, 3B, 3Ci-iv, 3D, 3E, and 3Fi-viii.

The first test of each vector can be a low-intensity stimulation, for example of 2.5 milliamps (mA) and 2 milliseconds (ms). In some embodiments, the first test of each vector can be a stimulation of 1 mA to 10 mA and 100 μs to 10 ms. After the first test of each vector, the stimulation system can determine whether the stable baseline is less than or equal to 2 and that there is no hemodynamic worsening. Stable baseline can refer to the variability of the signal, based on a combination of left ventricle end-diastolic pressure (LVEDP), LVP′min, and LVP′max. Stability can be defined by a composite index. Stable baseline of less than or equal to 2 can be confirmed when this composite index is less than or equal to 2. Hemodynamic worsening can refer to deterioration in the functioning of blood flow. Hemodynamic worsening can include hypotension, tachycardia, bradycardia, cardiogenic shock, pulmonary edema, myocardial infarction, and arrythmias. If the stable baseline is 2 or less and there is no hemodynamic worsening, the stimulation system can proceed with the second test. If there is pain or sensation during the first test, hemodynamic worsening, or stable baseline is greater than 2, the steps can be programmed at lower amplitudes.

The second test of each vector can be a medium stimulation, for example 5 mA and 2 mS. In some embodiments, the second test of each vector can be a stimulation of 1 mA to 20 mA and 100 μs to 10 ms. After the second test of each vector, the stimulation system can determine whether the stable baseline is less than or equal to 2 and that there is no hemodynamic worsening. If the stable baseline is 2 or less and there is no hemodynamic worsening, the stimulation system can proceed with the second test. If there is pain or sensation during the first test, hemodynamic worsening, or stable baseline is greater than 2, the steps can be programmed at lower amplitudes.

The third test of each vector can be a higher stimulation, for example 10 mA and 2 mS. In some embodiments, the third test of each vector can be a stimulation of 5 mA to 30 mA and 100 μs to 10 ms. After the third test of each vector, the stimulation system can determine whether the stable baseline is less than or equal to 2 and that there is no hemodynamic worsening.

Between the first vector test and the second vector test, there can be a break of less than 5 minutes. In some embodiments, there can be a break of 1 minute to 10 minutes. There can be two vector tests including three tests each. In some embodiments, there can be 1 vector test to 10 vector tests including 1 to 5 tests each. In some embodiments, there can be 1 vector test to 20 vector tests including 1 to 10 tests each. Vector tests can be continuously conducted until a satisfactory therapy efficacy is achieved.

Each vector test can be conducted using a different vector. In some embodiments, parameters varied between vector tests can include polarity, pulsing mode, pulse width, amplitude, frequency, conductance, phase, voltage, current, duration, inter-pulse interval, duty cycle, dwell time, sequence, wavelength, waveform, electrode combination, electrode configuration, or electrode position. The systems and methods herein can include measuring physiological parameters during the vector tests to determine the optimal polarity, pulsing mode, pulse width, amplitude, frequency, conductance, phase, voltage, current, duration, inter-pulse interval, duty cycle, dwell time, sequence, wavelength, waveform, electrode combination, electrode configuration, or electrode position for stimulation. The optimal parameters can be the parameters that result in the highest therapy efficacy.

Multiple electrodes can be engaged with the patient, for example as part of a neural stimulation system or an acutely implanted neural stimulation system or a chronically implantable system. Different electrodes or combinations of electrodes can be used to deliver stimulation without moving the electrodes. This can allow various vectors to be tested without needing to adjust the systems inside the patient.

In some examples, the vector tests can be conducted with CPNS therapy. In some implementations, the optimal parameters can be the parameters that force the restart of the local control circuit of the ICNS. In some implementations, the optimal parameters can be the parameters that cause the ICNS to shift from its maladaptive state, or the parameters that cause the ICNS to shift to a state with a therapy efficacy above a threshold.

During the vector tests, measurement of physiological parameters such as contractility, lusitropy, and/or chronotropy can be used to determine therapy efficacy, for example as described in FIG. 4. Therapy delivery can be adjusted based on the measurements of physiological parameters. In some embodiments, the parameters of stimulation can be automatically adjusted to the optimal parameters. In some embodiments, the stimulation system can present a user (e.g., clinical professional) with the optimal parameters, and the user can manually adjust the stimulation parameters.

FIGS. 6A, 6B, 6C, and 6D show an example of a decision diagram 600 for vector testing. The decision diagram 600 can be used to test for vectors with positive therapy efficacy as in FIGS. 4 and 5, for example using a stimulation system as described in FIG. 13.

At block 602, electrical stimulation can be delivered for 5 seconds, followed by electrical stimulation for 30 seconds. This test can have similar operating parameters to the tests of FIG. 5, for example the low stimulation test.

At block 604, the stimulation system can determine whether the patient experiences significant arrythmia or sensation. Significant arrythmia can include abnormal heart rhythm that can affect a patient's health. This can include a heart rate above or below a threshold, for example above 100 bpm or below 60 bpm. Sensation can include feeling or pain caused by the stimulation. The stimulation system can record the indications of arrythmia or sensation.

At block 606, the stimulation system can conduct a test at the next vector after the patient is at stable baseline of 2 or less for 5 minutes. The next vector can be the next configuration, combination, or position of electrodes. The next vector can be described at block 644.

At block 608, the stimulation system can determine whether there is a hemodynamic change or a significant hemodynamic change. A hemodynamic change can include a change in contractility, preload, lusitropy, systolic blood pressure, and/or chronotropy. The stimulation system can record the hemodynamic change.

At block 610, the stimulation system can determine whether goodness is greater than zero. Goodness can be calculated as described in FIGS. 7A, 7B, and 8. Goodness can be a positive therapy efficacy as described in FIG. 4.

At block 612, the stimulation system can determine that the therapeutic response has a positive therapy efficacy, or goodness. The stimulation system can record the response. The stimulation system can record the parameters and indicate that they are beneficial. The stimulation system can determine that the response is optimal if it has the highest therapy efficacy or goodness recorded.

At block 614, the stimulation system can measure the patient's heart rate, or stability as described in FIG. 5. The stimulation system can confirm that the patient's heart rate is at a stable baseline of 2 or less before proceeding.

At block 616, the stimulation system can continue to the next test with the same vector, as described at block 618.

At block 618, electrical stimulation can be delivered for 5 seconds, followed by electrical stimulation for 30 seconds. This test can have similar operating parameters to the tests of FIG. 5, for example the medium stimulation test.

At block 620, the stimulation system can determine whether the patient experiences significant arrythmia or sensation similar to block 604.

At block 622, the stimulation system can conduct a test at the next vector after the patient is at stable baseline similar to block 606.

At block 624, the stimulation system can determine whether there is a hemodynamic change or a significant hemodynamic change similar to block 608.

At block 626, the stimulation system can determine whether a goodness indicator value is greater than zero similar to block 610.

At block 628, the stimulation system can determine that the therapeutic response has a positive therapy efficacy or goodness indication similar to block 612.

At block 630, the stimulation system can measure that the patient's heart rate is at a stable baseline similar to block 614.

At block 632, the stimulation system can continue to the next test with the same vector, as described at block 634.

At block 634, electrical stimulation can be delivered for 5 seconds, followed by electrical stimulation for 30 seconds. This test can have similar operating parameters to the tests of FIG. 5, for example higher stimulation test.

At block 636, the stimulation system can determine whether the patient experiences significant arrythmia or sensation similar to block 604.

At block 638, whether goodness is greater than zero similar to block 610.

At block 640, the stimulation system can determine that the therapeutic response has a positive therapy efficacy or goodness similar to block 612.

At block 642, the stimulation system can conduct a test at the next vector after the patient is at stable baseline similar to block 606.

At block 644, the stimulation system can change the vector of the stimulation from the vector used for the stimulation at blocks 602, 618, and 634. Changing the vector can include changing the electrode configuration, combination, or position of the electrodes used for stimulation. In some embodiments, changing the vector can include changing the polarity, pulsing mode, pulse width, amplitude, frequency, conductance, phase, voltage, current, duration, inter-pulse interval, duty cycle, dwell time, sequence, wavelength, waveform, electrode combination, electrode configuration, or electrode position. Changing vectors include changing electrode configuration as described in FIGS. 3A, 3B, 3Ci-iv, 3D, 3E, and 3Fi-viii. The stimulation system can conduct any or all of the steps of FIGS. 6A and 6B with the next vector.

At block 646, the stimulation system can determine whether 30 minutes have passed since testing began. In some embodiments, the stimulation system can determine whether 10 minutes to 1 hour have passed since testing began. In some embodiments, the stimulation system can determine whether 1 to 5 hours have passed since testing began.

At block 648, the stimulation system can alert the user to consider repositioning if no response, or no response with positive therapeutic efficacy or goodness, has been detected. The stimulation system can alert the user to select another vector, wait 5 minutes, and start again. In some embodiments, the stimulation system can alert the user to select another vector, wait 1 minute to 20 minutes, and start again. The stimulation system can automatically change the vector to the next vector, wait, and start again.

At block 650, the stimulation system can determine that at least one response, or response with positive therapeutic efficacy or goodness, has been detected. The stimulation system can determine that an optimal therapeutic response has been detected.

At block 652, the stimulation system can extend testing time or end the testing. The stimulation system can initiate the protocol for no time remaining as described in FIGS. 9A and 9B.

At block 654, the stimulation system can recommend that the patient move to the patient room. In some embodiments, the stimulation system can record and/or display the optimal parameters for a positive therapeutic response. In some embodiments, the stimulation system can record and/or display all parameters with a positive therapeutic response. In some embodiments, the stimulation system can alert the intensive care unit as to the recorded parameters.

### Goodness Indicator

FIG. 7A shows a flow diagram 700 of an example for a multifactor efficacy indicator, or goodness indicator, using left ventricular pressure. The goodness indicator can receive input from sensors measuring physiological parameters of the patient. The sensors can simultaneously monitor physiological parameters such as contractility, preload, lusitropy, systolic function, and/or chronotropy. These parameters can be measured before, during, and/or after stimulation, for example CPNS therapy. For each measurement, the percentage change between the physiological parameter before stimulation and the physiological parameter during stimulation can be calculated and stratified. Goodness can be calculated as a combination of the changes in physiological parameters. Goodness can be higher when the changes in physiological changes are beneficial to the health of the patient. Goodness can be zero when the measurements do not meet a specified change from baseline.

Efficacy, or goodness, can be calculated in response to stimulation from different stimulation vectors. Different stimulation vectors can include different polarity, pulsing mode, pulse width, amplitude, frequency, conductance, phase, voltage, current, duration, inter-pulse interval, duty cycle, dwell time, sequence, wavelength, waveform, electrode combination, electrode configuration, or electrode position. The stimulation system can present a user with the goodness value of each vector, or the vector with the largest goodness value. The user can select the vector with the largest goodness value to optimize stimulation. Different vectors can include different combinations of electrodes as described in FIGS. 3A, 3B, 3Ci-iv, 3D, 3E, and 3Fi-viii.

Different cardiovascular parameters can be used to determine goodness. The inputs for the goodness indicator can include or consist essentially of one parameter related to contractility, one parameter related to preload, and one parameter related to heart rate. The cardiovascular parameters can be measured using sensors such as an electrocardiogram, a pulse oximeter, a contractility sensor, a pressure sensor, and combinations thereof.

In some embodiments, the example thresholds used in blocks 702-724 can be changed to patient-specific thresholds or alternate thresholds.

At block 702, the stimulation system can determine the percentage change in the cyclic maximum of the first derivative of left ventricular pressure of the patient. This measurement can be used as an indication of contractility. If the percentage change in the cyclic maximum of the first derivative of left ventricular pressure of the patient is less than a lower threshold, for example 1%, the stimulation system can determine that goodness equals zero.

At block 704, if the percentage change in the cyclic maximum of the first derivative of left ventricular pressure is greater than or equal to a first upper threshold, for example 10%, the stimulation system can determine the raw change in minimum left ventricular end-diastolic pressure of the patient. This measurement can be used as an indication of preload. If the raw change in minimum left ventricular end-diastolic pressure of the patient is greater than or equal to a threshold, for example 3 mmHg, the stimulation system can determine that goodness equals zero.

At block 706, if the raw change in minimum left ventricular end-diastolic pressure is less than a threshold, for example 3 mmHg at block 704 or 1 mmHg at block 714, the stimulation system can determine the percentage change in minimum left ventricular pressure. This measurement can be used as an indication of lusitropy. If the percentage change in minimum left ventricular pressure is less than a threshold, for example −5%, the stimulation system can determine that goodness equals zero.

At block 708, if the percentage change in the cyclic minimum of the first derivative of left ventricular pressure is greater than or equal to a threshold, for example −5%, the stimulation system can determine the percentage change in the cyclic maximum of the first derivative of arterial blood pressure. This measurement can be used as an indication of a second dimension of contractility. Measuring two dimensions of contractility can be helpful for determining goodness, or therapy efficacy. If the percentage change in the cyclic maximum of the first derivative of arterial blood pressure is less than a threshold, for example −5%, the stimulation system can determine that goodness equals zero.

At block 710, if the percentage change in the cyclic maximum of the first derivative of arterial blood pressure is greater than or equal to a threshold, for example −5%, the stimulation system can determine the percentage change in systolic blood pressure. This measurement can be used as an indication of systolic blood pressure. If the percentage change in systolic blood pressure is less than a threshold, for example −5%, the stimulation system can determine that goodness is zero.

At block 712, if the percentage change in systolic blood pressure is greater than or equal to a threshold, for example −5%, the stimulation system can determine the percentage change and coefficient of variation of heart rate. This measurement can be used as an indication of chronotropy. If the absolute value of the percentage change in heart rate is greater than or equal to a threshold, for example 10%, and if the absolute value of the percentage change in heart rate is greater than or equal to a threshold, for example, 2 times the coefficient of variation percentage, the stimulation system can determine that goodness is zero. If the absolute value of the percentage change in heart rate is less than a threshold, for example 10%, or if the absolute value of the percentage change in heart rate is less than a threshold, for example, 2 times the coefficient of variation percentage, the stimulation system can calculate goodness at block 726.

At block 714, if the percentage change in the cyclic maximum of the first derivative of the left ventricular pressure is greater than or equal to a second upper threshold, for example 5%, the stimulation system can determine the raw change in minimum left ventricular end-diastolic pressure of the patient. This measurement can be used as an indication of preload. If the raw change in minimum left ventricular end-diastolic pressure of the patient is greater than or equal to a threshold, for example 1 mmHg, the stimulation system can determine that goodness equals zero.

At block 716, if the percentage change in the cyclic maximum of the first derivative of the left ventricular pressure is greater than or equal to a third upper threshold, for example 1%, the stimulation system can determine the raw change in minimum left ventricular end-diastolic pressure of the patient. This measurement can be used as an indication of preload. If the raw change in minimum left ventricular end-diastolic pressure of the patient is greater than or equal to a threshold, for example 0 mmHg, the stimulation system can determine that goodness equals zero.

At block 718, if the raw change in minimum left ventricular end-diastolic pressure is less than a threshold, for example 0 mmHg, the stimulation system can determine the percentage change in the cyclic minimum of the first derivative of left ventricular pressure. This measurement can be used as an indication of lusitropy. If the percentage change in the cyclic minimum of the first derivative of left ventricular pressure is less than a threshold, for example −1%, the stimulation system can determine that goodness equals zero.

At block 720, if the percentage change in the cyclic minimum of the first derivative of left ventricular pressure is greater than or equal to a threshold, for example −1%, the stimulation system can determine the percentage change in the cyclic maximum of the first derivative of arterial blood pressure. This measurement can be used as an indication of a second dimension of contractility. Measuring two dimensions of contractility can be helpful for determining goodness, or therapy efficacy. If the percentage change in the cyclic maximum of the first derivative of arterial blood pressure is less than a threshold, for example −1%, the stimulation system can determine that goodness equals zero.

At block 722, if the percentage change in the cyclic maximum of the first derivative of arterial blood pressure is greater than or equal to a threshold, for example −1%, the stimulation system can determine the percentage change in systolic blood pressure. This measurement can be used as an indication of systolic blood pressure. If the percentage change in systolic blood pressure is less than a threshold, for example −1%, the stimulation system can determine that goodness is zero.

At block 724, if the percentage change in systolic blood pressure is greater than or equal to a threshold, for example −1%, the stimulation system can determine the percentage change and coefficient of variation of heart rate. This measurement can be used as an indication of chronotropy. If the absolute value of the percentage change in heart rate is greater than or equal to a threshold, for example 5%, and if the absolute value of the percentage change in heart rate is greater than or equal to a threshold, for example, 2 times the coefficient of variation percentage, the stimulation system can determine that goodness is zero. If the absolute value of the percentage change in heart rate is less than a threshold, for example 5%, or if the absolute value of the percentage change in heart rate is less than a threshold, for example, 2 times the coefficient of variation percentage, the stimulation system can calculate goodness at block 726.

At block 726, the stimulation system can calculate goodness (e.g., a goodness indicator value), and it can be defined as a sum of terms. Each term can be a physiological parameter multiplied by a coefficient. One term can be directly proportional to the percentage change in the cyclic maximum of the first derivative of left ventricular pressure, for example, with a coefficient of 2. A second term can be directly proportional to the raw change in minimum left ventricular end-diastolic pressure, for example, with a coefficient of −30. A third term can be directly proportional to the percentage change in the cyclic minimum of the first derivative of left ventricular pressure, for example, with a coefficient of 2. A fourth term can be directly proportional to the percentage change in the cyclic minimum of the first derivative of arterial blood pressure, for example, with a coefficient of 1. A fifth term can be directly proportional to the percentage change in systolic blood pressure, for example, with a coefficient of 1. A sixth term can be directly proportional to the absolute value of the percentage change in heart rate, for example, with a coefficient of −1.5. The coefficients of each physiological parameter can be adjusted based on the patient. Different physiological parameters can be used for different patients.

FIG. 7B shows a flow diagram 750 of an example for a goodness indicator using arterial blood pressure. The flow diagram 750 can be similar to the flow diagram 700 of FIG. 7A.

At block 752, the procedure can be similar to block 702. However, the stimulation system can measure percentage change in the cyclic maximum of the first derivative of the arterial blood pressure rather than the cyclic maximum of the first derivative of the left ventricular pressure.

At block 754, if the cyclic maximum of the first derivative of the arterial blood pressure is greater than or equal to a first upper threshold, for example 10%, the stimulation system can measure percentage change in mean arterial pressure. If the percentage change in mean arterial pressure is greater than or equal to a threshold, for example −5%, the stimulation system can determine that goodness is zero.

At block 756, the procedure can be similar to block 706. However, the stimulation system can measure the percentage change in pulse pressure rather than the percentage change in the cyclic minimum of the first derivative of the left ventricular pressure.

At block 758, the procedure can be similar to block 708. However, the stimulation system can measure the percentage change in heart rate times pulse pressure rather than the cyclic maximum of the first derivative of the arterial blood pressure.

At block 760, the procedure can be similar to block 710.

At block 762, the procedure can be similar to block 712.

At block 764, if the percentage change in the cyclic maximum of the first derivative of the arterial blood pressure is greater than or equal to a second upper threshold, for example 5%, the stimulation system can measure the percentage change in mean arterial pressure. If the percentage change in mean arterial pressure is greater than or equal to a threshold, for example −1%, the stimulation system can determine that goodness is zero.

At block 766, if the percentage change in the cyclic maximum of the first derivative of the arterial blood pressure is greater than or equal to a third upper threshold, for example 1%, the stimulation system can measure percentage change in mean arterial pressure. If the percentage change in mean arterial pressure is greater than or equal to a threshold, for example −1%, the stimulation system can determine that goodness is zero.

At block 768, the procedure can execute a step similar to block 718. However, the stimulation system can measure the percentage change in pulse pressure rather than percentage change in the cyclic minimum of the first derivative of the left ventricular pressure.

At block 770, the procedure can execute a step similar to block 720. However, the stimulation system can measure the percentage change in heart rate times pulse pressure rather than the percentage change in the cyclic maximum of the first derivative of the arterial blood pressure.

At block 772, the procedure can execute a step similar to block 722.

At block 774, the procedure can execute a step similar to block 724.

At block 776, the stimulation system can calculate goodness (e.g., a goodness indicator value), and can be defined as a sum of terms. Each term can be a physiological parameter multiplied by a coefficient. One term can be directly proportional to the percentage change in the cyclic maximum of the first derivative of arterial blood pressure, for example, with a coefficient of 1. A second term can be directly proportional to the percentage change in mean arterial pressure, for example, with a coefficient of 1. A third term can be directly proportional to the percentage change in pulse pressure, for example, with a coefficient of 1. A fourth term can be directly proportional to the percentage change in the heart rate times pulse pressure, for example, with a coefficient of 2. A fifth term can be directly proportional to the percentage change in systolic blood pressure, for example, with a coefficient of 1. A sixth term can be directly proportional to the absolute value of the percentage change in heart rate, for example, with a coefficient of −1.5. The coefficients of each physiological parameter can be adjusted based on the patient. Different physiological parameters can be used for different patients.

FIG. 8 illustrates an example of a goodness calculation, or multifactor efficacy calculation, using the left ventricular pressure of a patient. In one example, the percentage change in the cyclic maximum of the first derivative of the left ventricular pressure is 9.7%, the raw change in left ventricular end-diastolic pressure is 0.7 mmHg, the percentage change in the cyclic minimum of the first derivative of the left ventricular pressure is 7%, the percentage change in the cyclic maximum of the first derivative of the arterial blood pressure is 12.7%, the percentage change in systolic blood pressure is 5.5%, and the absolute value of the percentage change in heart rate is 2.2%. In this example, these measurements are rounded to calculate a goodness value of approximately 30. In this example, spaced out diagonal lines ascending to the right can indicate a most positive value, close together diagonal lines ascending to the right can indicate a positive value, medium spaced diagonal lines ascending to the left can indicate a less positive value, medium spaced diagonal lines ascending to the right can indicate an even less positive, or almost zero, value, and crossing diagonal lines can indicate a value for which goodness is zero. For example, contractility based on percentage change in the cyclic maximum of the first derivative of the left ventricular pressure of 10% or higher can be most positive, 5% or higher can be positive, 0% or higher can be less positive, and lower than 0% can be zero. Preload based on raw change in LVEDP of 3 mmHg or higher can be zero, 1 mmHg or higher can be less positive, and lower than 1 mmHg can be most positive. Lusitropy based on percentage change in the cyclic minimum of the first derivative of the left ventricular pressure of −1% or higher can be most positive, lower than −1% can be almost zero, and lower than −5% can be zero. Contractility based on change in the maximum of the first derivative of the arterial blood pressure of −1% or higher can be most positive, lower than −1% can be almost zero, and lower than −5% can be zero. Systolic blood pressure based on change in systolic blood pressure of −1% or higher can be most positive, lower than −1% can be almost zero, and lower than −5% can be zero. Chronotropy based on absolute change in maximum heart rate of 10% or higher can be zero, 5% or higher can be less positive, and lower than 5% can be positive.

The goodness value, or other multi-factor indicator, can be calculated for multiple vectors to determine the optimal vector. The optimal vector can be the vector that results in the highest goodness value or other multi-factor indicator indicative of therapeutic efficacy or likelihood thereof.

### Stimulation Protocols

FIG. 9A shows a flow diagram of an example 900 for the protocol for no time remaining when no untested vectors in the target zone remain. At block 902, the stimulation system can determine that tested vectors were undesirable at higher amplitudes. Higher amplitudes can include an amplitude of 10 mA. In some embodiments, higher amplitudes can include an amplitude of 5 mA to 30 mA. Tested vectors can be undesirable when goodness is zero, therapy efficacy is negative, hemodynamic change is negative, significant arrythmia occurs, or sensation occurs, as described in FIGS. 6A, 6B, 6C, 6D, 7A, 7B, and/or 8. The stimulation system can determine that no untested vectors remain in the target zone. For example, all electrode configurations may have been tested. In another example, all electrode configurations that were planned to be tested may have been tested.

At block 904, the stimulation system can review the data of the tests at lower amplitudes. For example, the stimulation system can review the data of tests at 2.5 mA and 5 mA to determine if the stimulation was safe. In some embodiments, the stimulation system can review the data of tests at 1-10 mA to determine if the stimulation was safe.

At block 906, the stimulation system can abort the case. Aborting the case can mean determining not to proceed with stimulation for the patient.

At block 908, the patient can proceed to the ICU. This procedure can be similar to block 654 of FIG. 6D.

FIG. 9B shows a flow diagram of an example 950 for the protocol for no time remaining when untested vectors in the target zone remain. At block 952, the stimulation system can determine that tested vectors were undesirable. Tested vectors can be undesirable when goodness is zero, therapy efficacy is negative, hemodynamic change is negative, significant arrythmia occurs, or sensation occurs, as described in FIGS. 6A, 6B, 6C, 6D, 7A, 7B, and/or 8. The stimulation system can determine that untested vectors remain in the target zone. For example, all electrode configurations may not have been tested. In another example, all electrode configurations that were planned to be tested may not have been tested.

At block 954, the patient can consider continuing safety testing target zone vectors or reposition. The stimulation system can continue testing other vectors or repositioning the electrodes. The stimulation system can abort the case if the physician recommends no stimulation.

At block 956, the first two tests of the vector test can be conducted on available vectors. In some embodiments, the one to three tests of the vector test can be conducted on available vectors. This can include testing at 2.5 mA and 5 mA. In some embodiments, the stimulation system can test at 1 mA to 10 mA.

At block 958, the stimulation system can determine whether there were undesirable effects. Tested vectors can have undesirable effects when goodness is zero, therapy efficacy is negative, hemodynamic change is negative, significant arrythmia occurs, or sensation occurs, as described in FIGS. 6A, 6B, 6C, 6D, 7A, 7B, and/or 8.

At block 960, the stimulation system can abort the case. Aborting the case can mean determining not to proceed with stimulation for the patient.

At block 962, the patient can proceed to the patient room. This procedure can be similar to block 654 of FIG. 6D.

FIG. 10 shows a flow diagram of an example 1000 for the patient room considerations for vector selection. At block 1002, the stimulation system can review catheterization laboratory data and determine the best vector to use. Catheterization laboratory data can include all vector testing data. The best or “optimal” vector can be the vector that results in the highest goodness value.

At block 1004, the stimulation system can evaluate the patient trajectory with a physician. This evaluation can occur daily. In some embodiments, the evaluation can occur every 1-6 days or weekly. Evaluating trajectory can include measuring the physiological parameters of the patient to determine well-being.

At block 1006, the patient can continue therapy for 24 hours. In some embodiments, the patients can continue therapy until the next evaluation.

At block 1008, the stimulation system can consider a different vector that was screened in a catheterization laboratory. The physician can decide which vector to use. The stimulation system can use the next most optimal vector. The stimulation system can begin stimulation therapy with the different vector.

At block 1010, the physician can consult with the patient to determine whether to continue stimulation therapy. In some embodiments, the stimulation system can determine whether the patient should continue stimulation therapy.

At block 1012, the stimulation system can abort or end the case. Aborting the case can mean determining not to proceed with stimulation for the patient.

FIG. 11 shows a flow diagram of an example 1100 for uptitration in the patient room. The stimulation system can stop stimulation immediately if sensation is intense, or at or above 3. The stimulation system can stop stimulation immediately if a major cardiac event occurs. The stimulation system can start weaning if central venous pressure is less than or equal to 4. In some embodiments, the stimulation system can start weaning if central venous pressure is less than or equal to 1 to 5.

At block 1102, the stimulation system can start stimulation at 1 mA with 1 minute on and 5 minutes off on the vector chosen for stimulation therapy. In some embodiments, the stimulation system can start stimulation at 0.5 mA to 5 mA with 1 minute to 5 minutes on and 3 minutes to 10 minutes off on the vector chosen for stimulation therapy.

At block 1104, the stimulation system can determine whether there is a hemodynamic change or a significant hemodynamic change. A hemodynamic change can include a change in contractility, preload, lusitropy, systolic blood pressure, electromechanical timings, and/or chronotropy. The stimulation system can record the hemodynamic change. The stimulation system can determine whether the patient experiences significant arrythmia or sensation. Significant arrythmia can include abnormal heart rhythm that can affect a patient's health. This can include a heart rate above or below a threshold, for example above 100 bpm or below 60 bpm. Sensation can include feeling or pain caused by the stimulation. The stimulation system can record the indications of arrythmia or sensation. Sensation above 2 can be determined to be a threshold.

At block 1106, the stimulation system can determine whether the test reached 7 mA. In some embodiments, the stimulation system can determine whether the test reached 3 mA to 10 mA.

At block 1108, the stimulation system can determine whether the cath lab goodness amplitude has been reached. The goodness amplitude can be the amplitude with optimal goodness.

At block 1110, the stimulation system can increase the amplitude of the stimulation by 1 mA and test for 1 minute on and 15 minutes off. In some embodiments, the stimulation system can increase the amplitude of the stimulation by 0.5 mA to 5 mA and test for 1 minute to 5 minutes on and 10 minutes to 20 minutes off.

At block 1112, the stimulation system can have the patient wait 30 minutes and stimulate with an amplitude of 0.5 mA less than the last tried amplitude. In some embodiments, the stimulation system can wait 10 minutes to 45 minutes and stimulate with an amplitude of 0.25 mA to 2 mA less than the last tried amplitude.

At block 1114, the stimulation system can determine whether there is a hemodynamic change or a significant hemodynamic change. A hemodynamic change can include a change in contractility, preload, lusitropy, systolic blood pressure, and/or chronotropy. The stimulation system can record the hemodynamic change. The stimulation system can determine whether the patient experiences significant arrythmia or sensation. Significant arrythmia can include abnormal heart rhythm that can affect a patient's health. This can include a heart rate above or below a threshold, for example above 100 bpm or below 60 bpm. Sensation can include feeling or pain caused by the stimulation. The stimulation system can record the indications of arrythmia or sensation. Sensation above 2 can be determined to be a threshold.

At block 1116, the stimulation system can have the patient wait 15 minutes, stimulate at the last used amplitude for 1 minute on and 10 minutes off for 2 hours on, and 2 hours off. This cycle can be repeated 5 times. In some embodiments, the stimulation system can have the patient wait 5 minutes to 40 minutes, stimulate at the last used amplitude for 1 minute to 10 minutes on and 5 minutes to 20 minutes off for 1 hour to 5 hours on, and 1 hour to 5 hours off. This cycle can be repeated 1 time to 10 times.

At block 1118, the stimulation system can monitor hemodynamics and rhythm for associations between stimulation and undesirable changes. Undesirable changes can include systolic blood pressure outside 90 to 165 mmHg, the cyclic maximum of the first derivative of the arterial blood pressure with a decrease or significant decrease, pulmonary artery pressure minimum with an increase or significant increase, heart rate with a significant change, pulse pressure with a decrease or significant decrease, systolic blood pressure with a decrease or significant decrease, a worsening ST depression, abnormal pressure waveforms, rhythm abnormalities, coughing, or central venous pressure of less than or equal to 4 mmHg, or, in some embodiments, 1 mm Hg to 10 mmHg.

At block 1120, the stimulation system can determine if an association between the stimulation and undesirable effect is present.

At block 1122, the stimulation system can determine that the patient is in the no sensation group. The stimulation system can include considering the potential reason why the patient is not feeling stimulation. This can include medication, nerve damage, incorrect placement, device malfunction, psychological factors, tolerance, or other factors.

At block 1124, the stimulation system can stimulate the patient at 5.5 mA for 1 minute on, 10 minutes off in a cycle of 2 hours on, 2 hours off. In some embodiments, the stimulation system can stimulate the patient at 0.5 mA to 10 mA for 1 minute to 5 minutes on, 5 minutes to 20 minutes off in a cycle of 1 hour to 4 hours on, 1 hour to 4 hours off.

At block 1126, the stimulation system can stimulate at 0.5 mA or 1 mA below the last tried amplitude. The stimulation system can stimulate at 0.5 mA less when the goodness amplitude is 2.5 mA.

At block 1128, the patient can discuss potential basket migration, basket dislodgement, change of vector, amplitude, or medication change.

### Therapy Weaning

FIG. 12 shows a flow chart 1200 of an example of therapy weaning. Therapy weaning can include gradually reducing or tapering stimulation intensity. The method can be conducted by a system as described herein, for example in FIG. 4.

Therapy weaning can be used in any system that delivers stimulation to a neural target. For example, therapy weaning can be used in acutely implanted neural stimulation systems, chronically implanted neural stimulation systems, and non-implantable neural stimulation systems, such as a transcutaneous neural stimulator.

Advantageously, therapy weaning can reduce side effects and adverse events due to stimulation intensity, reduce battery use, increase longevity of stimulation devices (for example chronically implantable devices), and reduce habituation of the therapeutic response.

The dose off time for therapy weaning can be 15 minutes. In some embodiments, the dose off time for therapy weaning can be 5 minutes to 45 minutes (e.g., 5 minutes to 15 minutes, 10 minutes to 30 minutes, 15 minutes to 45 minutes, overlapping ranges thereof, or any value within the recited ranges). Therapy wean time can be set to 3 hours and the type can be a duty cycle. In various implementations, therapy wean time can be set to 1 hour to 10 hours (e.g., 1 hour to 3 hours, 2 hours to 4 hours, 3 hours to 6 hours, 5 hours to 10 hours, overlapping ranges thereof, or any value within the recited ranges). Stimulation with therapy weaning can be stopped after 2 hours. In various implementations, stimulation with therapy weaning can be stopped after 1 hour to 5 hours (e.g., 1 hour to 3 hours, 2 hours to 4 hours, 3 hours to 5 hours, overlapping ranges thereof, or any value within the recited ranges). The goal of therapy weaning can be returning the ICNS or CANS to homeostasis.

At block 1202, the stimulation system can deliver stimulation to the neural target. The neural target can be the cardiac plexus, for example at the level of the right pulmonary artery. The neural target can be another area of the heart or of another organ.

At block 1204, the stimulation system can reduce the stimulation intensity. The stimulation intensity can include stimulation amplitude, frequency, pulse width, and duty cycle. Stimulation intensity can be reduced after a period of time following confirmation of therapeutic response. The duty cycle can be gradually reduced, in that the stimulation on-time is reduced or that the stimulation off-time is increased.

At block 1206, the stimulation system can determine the therapeutic response. Evaluation of therapeutic response can occur periodically. Therapy weaning can be reduced or suspended upon detection of reduced therapeutic efficacy. Therapeutic efficacy can be determined based on the goodness indicator described herein, for example in FIGS. 7A, 7B, and 8.

At block 1208, if the therapeutic response is above a threshold, the stimulation system can continue therapy weaning. The stimulation system can continue reducing stimulation intensity over the duration of the therapy weaning (either continuously gradually or in discrete increments).

At block 1210, if the therapeutic response is below a threshold, the stimulation system can reduce or suspend therapy weaning. The stimulation system can stop reducing stimulation intensity or reduce stimulation intensity by a smaller percentage.

FIG. 13 illustrates an example of a stimulation system 1301. As shown in FIG. 13, the stimulation system 1301 includes an input/output connector 1303 that can releasably join the conductive elements of the catheter, conductive elements of a second catheter, and/or sensors for sensing the one or more cardiac properties from the skin surface of the patient, as discussed herein. An input from the sensor can also be releasably coupled to the input/output connector so as to receive the sensor signal(s) discussed herein. The conductive elements and/or sensors may be permanently coupled to the stimulation system (e.g., not releasably coupled).

The input/output connector 1303 is connected to an analog to digital converter 1305. The output of the analog to digital converter 1305 is connected to a microprocessor 1307, or multiple microprocessors or other processors, through a peripheral bus 1309 including, for example, address, data, and control lines. The microprocessor 1307 can process the sensor data, when present, in different ways depending on the type of sensor in use. The microprocessor 1307 can also control, as discussed herein, the pulse control output generator 1311 that delivers the stimulation electrical energy (e.g., electrical pulses) to the one or more electrodes via the input/output connector 1303 and/or housing 1323.

The parameters of the stimulation electrical energy (e.g., properties of the electrical pulses) can be controlled and adjusted, if desired, by instructions programmed in a memory 1313 and executed by a programmable pulse generator 1315. The memory 1313 may comprise a non-transitory computer-readable medium. The memory 1313 may include one or more memory devices capable of storing data and allowing any storage location to be directly accessed by the microprocessor 1307, such as random access memory (RAM), flash memory (e.g., non-volatile flash memory), and the like. The stimulation system 1301 may comprise a storage device, such as one or more hard disk drives or redundant arrays of independent disks (RAID), for storing an operating system and other related software, and for storing application software programs, which may be the memory 1313 or a different memory. The instructions in memory 1313 for the programmable pulse generator 1315 can be set and/or modified based on input from the sensors and the analysis of the one or more heart activity properties via the microprocessor 1307. The instructions in memory 1313 for the programmable pulse generator 1315 can also be set and/or modified through inputs from a professional via an input 1317 connected through the peripheral bus 1309. Examples of such an input include a keyboard and/or a mouse (e.g., in conjunction with a display screen), a touch screen, etc. A wide variety of input/output (I/O) devices may be used with the stimulation system 1301. Input devices include, for example, keyboards, mice, trackpads, trackballs, microphones, and drawing tablets. Output devices include, for example, video displays, speakers, and printers. The I/O devices may be controlled by an I/O controller. The I/O controller may control one or more I/O devices. An I/O device may provide storage and/or an installation medium for the stimulation system 1301. The stimulation system 1301 may provide USB connections to receive handheld USB storage devices. The stimulation system 1301 optionally includes a communications port 1319 that connects to the peripheral bus 1309, where data and/or programming instructions can be received by the microprocessor 1307 and/or the memory 1313.

Input from the input 1317 (e.g., from a professional), the communications port 1319, and/or from the one or more heart activity properties via the microprocessor 1307 can be used to change (e.g., adjust) the parameters of the stimulation electrical energy (e.g., properties of the electrical pulses). The stimulation system 1301 optionally includes a power source 1321. The power source 1321 can be a battery or a power source supplied from an external power supply (e.g., an AC/DC power converter coupled to an AC source). The stimulation system 1301 optionally includes a housing 1323.

The microprocessor 1307 can execute one or more algorithms in order to provide stimulation and/or to provide vector testing and titration such as described herein. The microprocessor 1307 can also be controlled by a professional via the input 1317 to initiate, terminate, and/or change (e.g., adjust) the properties of the electrical pulses. The microprocessor 1307 can execute one or more algorithms to conduct the analysis of the one or more heart activity properties sensed in response to the one or more electrical pulses delivered using the hierarchy of electrode configurations and/or the hierarchy of each property of the one or more electrical pulses, for example to help identify an electrode configuration and/or the property of the one or more electrical pulses delivered to the patient's heart. Such analysis and adjustments can be made using process control logic (e.g., fuzzy logic, negative feedback, etc.) so as to maintain control of the pulse control output generator 1311.

In some examples, the stimulation is operated with closed loop feedback control. In some examples, input is received from a closed-looped feedback system via the microprocessor 1307. The closed loop feedback control can be used to help maintain one or more of a patient's cardiac parameters at or within a threshold value or range programmed into memory 1313. For example, under closed loop feedback control measured cardiac parameter value(s) can be compared and then it can be determined whether or not the measured value(s) lies outside a threshold value or a pre-determined range of values. If the measured cardiac parameter value(s) do not fall outside of the threshold value or the pre-determined range of values, the closed loop feedback control continues to monitor the cardiac parameter value(s) and repeats the comparison on a regular interval. If, however, the cardiac parameter value(s) from a sensor indicate that one or more cardiac parameters are outside of the threshold value or the pre-determined range of values one or more of the parameters of the stimulation electrical energy will be adjusted by the microprocessor 1307.

The stimulation system 1301 may comprise one or more additional components, for example a display device, a cache memory (e.g., in communication with the microprocessor 1307), logic circuitry, signal filters, a secondary or backside bus, local buses, local interconnect buses, and the like. The stimulation system 1301 may support any suitable installation device, such as a CD-ROM drive, a CD-R/RW drive, a DVD-ROM drive, tape drives of various formats, USB device, hard-drive, communication device to a connect to a server, or any other device suitable for installing software and programs. The stimulation system 1301 may include a network interface to interface to a Local Area Network (LAN), Wide Area Network (WAN), or the Internet through a variety of connections including, but not limited to, standard telephone lines, LAN or WAN links, broadband connections, wireless connections (e.g., Bluetooth, Wi-Fi), combinations thereof, and the like. The network interface may comprise a built-in network adapter, network interface card, wireless network adapter, USB network adapter, modem, or any other device suitable for interfacing the stimulation system 1301 to any type of network capable of communication and performing the operations described herein. In some examples, the stimulation system 1301 may comprise or be connected to multiple display devices, which may be of the same or different in type and/or form. As such, any of the I/O devices and/or the I/O controller may comprise any type and/or form of suitable hardware, software, or combination of hardware and software to support, enable, or provide for the connection and use of multiple display devices by the stimulation system 1301. The stimulation system can interface with any workstation, desktop computer, laptop or notebook computer, server, handheld computer, mobile telephone, any other computer, or other form of computing or telecommunications device that is capable of communication and that has sufficient processor power and memory capacity to perform the operations described herein and/or to communication with the stimulation system 1301. The arrows shown in FIG. 13 generally depict the flow of current and/or information, but current and/or information may also flow in the opposite direction depending on the hardware.

Analysis, determining, adjusting, and the like described herein may be closed loop control or open loop control. For example, in closed loop control, a stimulation system may analyze a heart activity property and adjust an electrical signal property without input from a user. For another example, in open loop control, a stimulation system may analyze a heart activity property and prompt action by a user to adjust an electrical signal property, for example providing suggested adjustments or a number of adjustment options.

In some examples, a method of non-therapeutic calibration comprises positioning an electrode in a pulmonary artery of a heart and positioning a sensor in a right ventricle of the heart. The system further comprises delivering, via a stimulation system, a first series of electrical signals to the electrode. The first series comprises a first plurality of electrical signals. Each of the first plurality of electrical signals comprises a plurality of parameters. Each of the first plurality of electrical signals of the first series only differs from one another by a magnitude of a first parameter of the plurality of parameters. The method further comprises, after delivering the first series of electrical signals to the electrode, delivering, via the stimulation system, a second series of electrical signals to the electrode. The second series comprises a second plurality of electrical signals. Each of the second plurality of electrical signals comprises the plurality of parameters. Each of the second plurality of electrical signals of the second series only differs from one another by a magnitude of a second parameter of the plurality of parameters. The second parameter is different than the first parameter. The method further comprises determining, via the sensor, sensor data indicative of one or more non-electrical heart activity properties in response to delivering the first series of electrical signals and the second series of electrical signals. The method further comprises determining a therapeutic neuromodulation signal to be delivered to the pulmonary artery using selected electrical parameters. The selected electrical parameters comprise a selected magnitude of the first parameter and a selected magnitude of the second parameter. The selected magnitudes of the first and second parameters are based at least partially on the sensor data.

In some examples, a method of non-therapeutic calibration comprises delivering a first electrical signal of a series of electrical signals to an electrode in a first anatomical location and, after delivering the first electrical signal, delivering a second electrical signal of the series of electrical signals to the electrode. The second electrical signal differs from the first electrical signal by a magnitude of a first parameter of a plurality of parameters. The method further comprises sensing, via a sensor in a second anatomical location different than the first anatomical location, sensor data indicative of one or more non-electrical heart activity properties in response to the delivery of the series of electrical signals, and determining a therapeutic neuromodulation signal to be delivered to the first anatomical location using selected electrical parameters. The selected electrical parameters comprise a selected magnitude of the first parameter. The selected magnitude of the first parameter is based at least partially on the sensor data.

In some examples, the stimulation system can be used to help identify a preferred location for the position of the one or more electrodes along the posterior, superior and/or inferior surfaces of the main pulmonary artery, left pulmonary artery, and/or right pulmonary artery. To this end, the one or more electrodes of the catheter or catheter system are introduced into the patient and tests of various locations along the posterior, superior and/or inferior surfaces of the vasculature using the stimulation system are conducted so as to identify a preferred location for the electrodes. During such a test, the stimulation system can be used to initiate and adjust the parameters of the stimulation electrical energy (e.g., electrical current or electrical pulses). Such parameters include, but are not limited to, terminating, increasing, decreasing, or changing the rate or pattern of the stimulation electrical energy (e.g., electrical current or electrical pulses). The stimulation system can also deliver stimulation electrical energy (e.g., electrical current or electrical pulses) that is episodic, continuous, phasic, in clusters, intermittent, upon demand by the patient or medical personnel, or preprogrammed to respond to a signal, or portion of a signal, sensed from the patient.

An open-loop or closed-loop feedback mechanism may be used in conjunction with the present disclosure. For the open-loop feedback mechanism, a professional can monitor cardiac parameters and changes to the cardiac parameters of the patient. Based on the cardiac parameters the professional can adjust the parameters of the electrical current applied to autonomic cardiopulmonary fibers. Non-limiting examples of cardiac parameters monitored include arterial blood pressure, central venous pressure, capillary pressure, systolic pressure variation, blood gases, cardiac output, systemic vascular resistance, pulmonary artery wedge pressure, gas composition of the patient's exhaled breath and/or mixed venous oxygen saturation. Cardiac parameters can be monitored by an electrocardiogram, invasive hemodynamics, an echocardiogram, or blood pressure measurement or other devices known in the art to measure cardiac function. Other parameters such as body temperature and respiratory rate can also be monitored and processed as part of the feedback mechanism.

In a closed-loop feedback mechanism, the cardiac parameters of the patient are received and processed by the stimulation system, where the parameters of the electrical current are adjusted based at least in part on the cardiac parameters. As discussed herein, a sensor is used to detect a cardiac parameter and generate a sensor signal. The sensor signal is processed by a sensor signal processor, which provides a control signal to a signal generator. The signal generator, in turn, can generate a response to the control signal by activating or adjusting one or more of the parameters of the electrical current applied by the catheter to the patient. The control signal can initiate, terminate, increase, decrease or change the parameters of the electrical current. It is possible for the one or more electrodes of the catheter to be used as a sensor a recording electrode. When necessary, these sensing or recording electrodes may deliver stimulation electrical energy (e.g., electrical current or electrical pulses) as discussed herein.

The stimulation system can also monitor to determine if the one or more electrodes have dislodged from their position within the right pulmonary artery. For example, impedance values can be used to determine whether the one or more electrodes have dislodged from their position within the right pulmonary artery. If changes in the impedance values indicate that the one or more electrodes have dislodged from their position within the right pulmonary artery, a warning signal is produced by the stimulation system and the electrical current is stopped.

In several examples, the catheters provided herein include a plurality of electrodes, which includes two or more electrodes. It is understood that the phrase “a plurality of electrodes” can be replaced herein with two or more electrodes if desired. For the various examples of catheters and systems disclosed herein, the electrodes can have a variety of configurations and sizes. For example, the electrodes discussed herein can be ring-electrodes that fully encircle the body on which they are located. The electrodes discussed herein can also be a partial ring, where the electrode only partially encircles the body on which they are located. For example, the electrodes can be partial ring electrodes that preferably only contact the luminal surface of the main pulmonary artery and/or pulmonary arteries, as discussed herein. This configuration may help to localize the stimulation electrical energy, as discussed herein, into the vascular and adjacent tissue structures (e.g., autonomic fibers) and away from the blood. The electrodes and conductive elements provided herein can be formed of a conductive biocompatible metal or metal alloy. Examples of such conductive biocompatible metal or metal alloys include, but are not limited to, titanium, platinum or alloys thereof. Other biocompatible metal or metal alloys are known.

For the various examples, the elongate body of the catheters provided herein can be formed of a flexible polymeric material. Examples of such flexible polymeric material include, but are not limited to, medical grade polyurethanes, such as polyester-based polyurethanes, polyether-based polyurethanes, and polycarbonate-based polyurethanes; polyamides, polyamide block copolymers, polyolefins such as polyethylene (e.g., high density polyethylene); and polyimides, among others.

Each of the catheters and/or catheter systems discussed herein can further include one or more reference electrodes positioned proximal to the one or more electrodes present on the elongate body. These one or more reference electrodes can each include insulated conductive leads that extend from the catheter and/or catheter system so as to allow the one or more reference electrodes to be used as common or return electrodes for electrical current that is delivered through one or more of the one or more electrodes on the elongate body of the catheter and/or catheter system.

With respect to treating cardiovascular medical conditions, such medical conditions can involve medical conditions related to the components of the cardiovascular system such as, for example, the heart and aorta. Non-limiting examples of cardiovascular conditions include post-infarction rehabilitation, shock (hypovolemic, septic, neurogenic), valvular disease, heart failure including acute heart failure, angina, microvascular ischemia, myocardial contractility disorder, cardiomyopathy, hypertension including pulmonary hypertension and systemic hypertension, orthopnea, dyspnea, orthostatic hypotension, dysautonomia, syncope, vasovagal reflex, carotid sinus hypersensitivity, pericardial effusion, and cardiac structural abnormalities such as septal defects and wall aneurysms.

In some examples, a catheter, for example as discussed herein, can be used in conjunction with a pulmonary artery catheter, such as a Swan-Ganz type pulmonary artery catheter, to deliver transvascular neuromodulation via the pulmonary artery to an autonomic target site to treat a cardiovascular condition. In certain such examples, the catheter (or catheters) is housed within one of the multiple lumens of a pulmonary artery catheter.

In addition to the catheter and catheter system of the present disclosure, one or more sensing electrodes can be located on or within the patent. Among other things, the sensing electrodes can be used to detect signals indicting changes in various cardiac parameters, where these changes can be the result of the pulse of stimulation electrical energy delivered to stimulate the nerve fibers (e.g., autonomic nerve fibers) surrounding the main pulmonary artery and/or one or both of the pulmonary arteries. Such parameters include, but are not limited to, the patient's heart rate (e.g., pulse), among other parameters. The sensing electrodes can also provide signals indicting changes in one or more electrical parameter of vasculature (electrical activity of the cardiac cycle). Such signals can be collected and displayed, as are known, using known devices (e.g., electrocardiography (ECG) monitor) or a stimulation system, as discussed herein, which receives the detected signals and provides information about the patient.

Other sensors can also be used with the patient to detect and measure a variety of other signals indicting changes in various cardiac parameters. Such parameters can include, but are not limited to, blood pressure, blood oxygen level and/or gas composition of the patient's exhaled breath. For example, catheter and catheter system of the present disclosure can further include a pressure sensor positioned within or in-line with the inflation lumen for the inflatable balloon. Signals from the pressure sensor can be used to both detect and measure the blood pressure of the patient. Alternatively, the catheter and catheter system of the present disclosure can include an integrated circuit for sensing and measuring blood pressure and/or a blood oxygen level. Such an integrated circuit can be implemented using 0.18 μm CMOS technology. The oxygen sensor can be measured with optical or electrochemical techniques as are known. Examples of such oxygen sensors include reflectance or transmissive pulse oximetry those that use changes in absorbance in measured wavelengths optical sensor to help determined a blood oxygen level. For these various examples, the elongate body of the catheter can include the sensor (e.g., a blood oxygen sensor and/or a pressure sensor) and a conductive element, or elements, extending through each of the elongate body, where the conductive element conducts electrical signals from the blood oxygen sensor and/or the pressure sensor.

The detected signals can also be used by the stimulation system to provide stimulation electrical energy in response to the detected signals. For example, one or more of these signals can be used by the stimulation system to deliver the stimulation electrical energy to the one or more electrodes of the catheter or catheter system. So, for example, detected signals from the patent's cardiac cycle (e.g., ECG waves, wave segments, wave intervals or complexes of the ECG waves) can be sensed using the sensing electrodes and/or timing parameter of the subject's blood pressure. The stimulation system can receive these detected signals and based on the features of the signal(s) generate and deliver the stimulation electrical energy to the one or more electrode of the catheter or catheter system. As discussed herein, the stimulation electrical energy is of sufficient current and potential along with a sufficient duration to stimulate one or more of the nerve fibers surrounding the main pulmonary artery and/or one or both of the pulmonary arteries so as to provide neuromodulation to the patient.

FIG. 14A is a plot of contractility versus stimulation. Starting from a baseline contractility, the stimulation is turned ON for Time 1. There is some time delay for the stimulation to result in a change in contractility (e.g., about 10 to 20 seconds), after which contractility steadily climbs until reaching a fairly steady state. When contractility is turned OFF in time 2, there is some time delay before the contractility begins to decay. The decay delay when stimulation is OFF is longer than the delay when stimulation is ON. The time to ramp up to a baseline level during the decay is also less than from a baseline. The decay may also be reduced over time. Accordingly, the stimulation ON and OFF do not perfectly correlate to the durations when contractility changes.

In some examples, stimulation is turned ON for 5 seconds, followed by stimulation being turned OFF for 10 seconds. In some examples, stimulation is turned ON for 2 seconds, followed by stimulation being turned OFF for 5 seconds. In some examples, stimulation is turned ON for 10 seconds, followed by stimulation being turned OFF for 30 seconds. In some examples, stimulation is turned ON until a substantially steady state is achieved, followed by stimulation being turned OFF until a certain contractility is reached, at which point the stimulation is turned ON until the substantially steady state is again achieved, etc. Such an approach can reduce or minimize an effective dose. A duty cycle approach in view of this discovery can reduce the amount of time that stimulation is ON, which can reduce energy usage, maintain therapeutic effect, and/or reduce side effects, which can increase patient comfort and tolerability.

In some examples, a ramping feature could be used to slowly ramp the intensity of the stimulation ON and OFF, or to shut the stimulation OFF quickly. A ramping feature can allow the patient to adapt to stimulation and reduce sudden transitions. For example, at least one parameter (e.g., ON duration, amplitude, pulse width, frequency, etc.) could be slowly increased and/or decreased over time until building towards a final value.

In some examples, for example for short term treatment, a duty cycle may comprise alternating ON for 5 seconds and OFF for 5 seconds for 1 hour. In some examples, for example for short term treatment, a duty cycle may comprise alternating ON for 5 seconds and OFF for 10 seconds for 1 hour. In some examples, for example for short term treatment, a duty cycle may comprise alternating ON for 10 minutes and OFF for 50 minutes for 1 hour. In some examples, for example for long term treatment, a duty cycle may comprise alternating ON for 1 hour and OFF for 1 hour for 1 day. In some examples, for example for long term treatment, a duty cycle may comprise alternating ON for 1 hour and OFF for 1 hour for 1 day. In some examples, for example for long term treatment, a duty cycle may comprise alternating ON for 1 hour and OFF for 23 hours for 1 day. The ON durations in long term treatment may include the cycling of the short-term treatments. For example, if alternating ON for 1 hour and OFF for 1 hour for 1 day, the durations in which stimulation is ON for 1 hour may comprise alternating ON for 5 seconds and OFF for 5 seconds for that 1 hour. In some examples, a plurality of different ON/OFF cycles may be used during a long term ON duration, for example 10 seconds ON and 10 seconds OFF for 1 minute, then 1 minute ON and 5 minutes OFF for 10 minutes, then 10 minutes ON and 50 minutes OFF for 4 hours, for a long term ON duration of 4 hours and 11 minutes. Short term and/or long term ON/OFF cycles may be at least partially based on a patient state (e.g., awake or sleeping, laying down or upright, time since initial stimulation, etc.).

FIG. 14B is a plot of contractility versus stimulation using a threshold-based approach and an optimized duty cycle. Stimulation is turned ON and OFF for some duration. As noted above, the decay of contractility after the duration is reduced such that contractility remains above a threshold for a certain duration. This duration may be known or determined, for example by sensing contractility. The broken line in FIG. 14B shows a time when the determination is made to restart the stimulation cycle for another duration. This process may be repeated for the time that the subject is being treated, until a recalibration, etc.

Certain procedures described herein may be divided between users at a catheter lab and an intensive care unit or subject's room. A catheter lab may deploy the device in a subject. A catheter lab may perform therapy titration (e.g., determining stimulation parameters for a maximum tolerable contractility and/or relaxation increase, determining stimulation parameters for a contractility and/or relaxation increase greater than a minimum value, determining stimulation parameters for a contractility and/or relaxation increase less greater than a maximum value, determining stimulation parameters for a heart rate increase less than a maximum value, etc.). An intensive care unit and/or subject's room may apply therapy at pre-established parameters. An intensive care unit and/or subject's room may monitor therapy (e.g., via ECG, BP/MAP, SvO2, change in contractility and/or relaxation, change in pressure, heart rate, etc.). An intensive care unit and/or subject's room may perform initial and/or follow-up (e.g., as needed) therapy titration (e.g., determining stimulation parameters for a maximum tolerable contractility and/or relaxation increase, determining stimulation parameters for a contractility and/or relaxation increase greater than a minimum value, determining stimulation parameters for a contractility and/or relaxation increase less greater than a maximum value, determining stimulation parameters for a heart rate increase less than a maximum value, etc.). An intensive care unit and/or subject's room may perform therapy ramp down. Some functions may be performed at any location as appropriate. For example, follow-up titration therapy may be performed by a catheter lab, which may be more experienced at establishing stimulation parameters.

### CLAUSES

Clause 1. A method for optimizing neurostimulation therapy, the method comprising: measuring a cardiovascular parameter at a first point in time; applying stimulation at a vector to a nerve proximal to a pulmonary artery; measuring the cardiovascular parameter at a second point in time; calculating a change in the cardiovascular parameter; and determining therapy efficacy of the stimulation at the vector based on the change in the cardiovascular parameter.

Clause 2. The method of clause 1, further comprising: measuring the cardiovascular parameter at a third point in time; applying stimulation at a second vector to the nerve proximal to the pulmonary artery; measuring the cardiovascular parameter at a fourth point in time; calculating a change in the cardiovascular parameter; and determining therapy efficacy of the stimulation at the second vector based on the change in the cardiovascular parameter.

Clause 3. The method of clause 2, further comprising applying the stimulation at the vector with higher therapy efficacy.

Clause 4. The method of clause 1, wherein the cardiovascular parameter comprises contractility, preload, and/or chronotropy.

Clause 5. The method of clause 1, wherein the vector comprises at least one of electrode position, electrode configuration, or electrode combination.

Clause 6. A method for optimizing neurostimulation therapy, the method comprising: measuring a plurality of cardiovascular parameters at a first point in time, the plurality of cardiovascular parameters comprising at least two of contractility, preload, and chronotropy; applying stimulation to a nerve at a vector; measuring the cardiovascular parameters at a second point in time; calculating changes in the plurality of cardiovascular parameters; and determining therapy efficacy based on the changes in the plurality of cardiovascular parameters.

Clause 7. The method of clause 6, wherein the cardiovascular parameters further comprise lusitropy and systolic blood pressure.

Clause 8. The method of clause 6, wherein measuring contractility comprises measuring at least two dimensions of contractility.

Clause 9. A method for optimizing neurostimulation therapy, the method comprising: applying a first stimulation to a nerve proximal to a pulmonary artery; measuring a cardiovascular parameter at a first point in time; applying a second stimulation to the nerve in the pulmonary artery with a reduced intensity; measuring the cardiovascular parameter at a second point in time; calculating a change in the cardiovascular parameter; and determining a therapeutic response of the second stimulation based on the change in the cardiovascular parameter.

Clause 10. The method of clause 9, further comprising: upon detecting the therapeutic response above a threshold, applying a third stimulation to the nerve in the pulmonary artery with a further reduced intensity.

Clause 11. The method of clause 9, further comprising: upon detecting the therapeutic response below a threshold, applying a third stimulation with a less reduced intensity.

Clause 12. The method of clause 9, further comprising: upon detecting the therapeutic response below a threshold, stopping applying stimulation.

Clause 13. A method of treating heart failure comprising: applying stimulation to one or more nerves of a cardiac plexus via one or more electrodes positioned within a right pulmonary artery of a subject; wherein the stimulation comprises pulses having a frequency between 2 Hz and 40 Hz, a pulse width of 100 μs to 4 ms and an intensity of 0.1 mA to 20 mA, wherein the stimulation is applied for a duration of 20 seconds to 120 seconds at intervals of 5 minutes to 20 minutes according to a sequence.

Clause 14. The method of clause 13, wherein the sequence is repeated for 2 hours and then stopped for 2 hours according to a cycle.

Clause 15. The method of clause 14, wherein the cycle is repeated for 48 hours.

Clause 16. The method of clause 14, wherein the intensity of the stimulation over a final weaning cycle is gradually reduced.

Clause 17. A system for treating heart failure comprising: a catheter comprising a plurality of electrodes; a stimulator comprising a pulse generator configured to deliver stimulation pulses to one or more of the plurality of electrodes of the catheter, wherein the stimulation pulses have a frequency of between 2 Hz and 40 Hz, a pulse width of 100 μs to 4 ms, and an intensity of 0.1 mA to 20 mA,

Clause 18. The system of clause 17, wherein trains of stimulation pulses are configured to be delivered for 20 seconds to 120 seconds and then stopped for 5 minutes to 20 minutes according to a sequence.

Clause 19. The system of clause 17, wherein the sequence is repeated for 2 hours and then stopped for 2 hours according to a cycle.

Clause 20. The system of clause 17, wherein the cycle is repeated for 48 hours.

The foregoing description and examples has been set forth merely to illustrate the disclosure and are not intended as being limiting. Each of the disclosed aspects and examples of the present disclosure may be considered individually or in combination with other aspects, examples, and variations of the disclosure. In addition, unless otherwise specified, none of the steps of the methods of the present disclosure are confined to any particular order of performance. Modifications of the disclosed examples incorporating the spirit and substance of the disclosure may occur to persons skilled in the art and such modifications are within the scope of the present disclosure. The headings used herein are merely provided to enhance readability and are not intended to limit the scope of the embodiments disclosed in a particular section to the features or elements disclosed in that section. The features or elements from one embodiment of the disclosure can be employed by other embodiments of the disclosure. For example, features described in one figure may be used in conjunction with embodiments illustrated in other figures.

While the methods and devices described herein may be susceptible to various modifications and alternative forms, specific examples thereof have been shown in the drawings and are herein described in detail. It should be understood, however, that the inventions are not to be limited to the particular forms or methods disclosed, but, to the contrary, the inventions to cover all modifications, equivalents, and alternatives falling within the spirit and scope of the various examples described and the appended claims. Further, the disclosure herein of any particular feature, aspect, method, property, characteristic, quality, attribute, element, or the like in connection with an example can be used in all other examples set forth herein. Any methods disclosed herein need not be performed in the order recited. Depending on the example, one or more acts, events, or functions of any of the algorithms, methods, or processes described herein can be performed in a different sequence, can be added, merged, or left out altogether (e.g., not all described acts or events are necessary for the practice of the algorithm). In some examples, acts or events can be performed concurrently, e.g., through multi-threaded processing, interrupt processing, or multiple processors or processor cores or on other parallel architectures, rather than sequentially. Further, no element, feature, block, or step, or group of elements, features, blocks, or steps, are necessary or indispensable to each example. Additionally, all possible combinations, subcombinations, and rearrangements of systems, methods, features, elements, modules, blocks, and so forth are within the scope of this disclosure. The use of sequential, or time-ordered language, such as “then,” “next,” “after,” “subsequently,” and the like, unless specifically stated otherwise, or otherwise understood within the context as used, is generally intended to facilitate the flow of the text and is not intended to limit the sequence of operations performed. Thus, some examples may be performed using the sequence of operations described herein, while other examples may be performed following a different sequence of operations.

The various illustrative logical blocks, modules, processes, methods, and algorithms described in connection with the examples disclosed herein can be implemented as electronic hardware, computer software, or combinations of both. To clearly illustrate this interchangeability of hardware and software, various illustrative components, blocks, modules, operations, and steps have been described above generally in terms of their functionality. Whether such functionality is implemented as hardware or software depends upon the particular application and design constraints imposed on the overall system. The described functionality can be implemented in varying ways for each particular application, but such implementation decisions should not be interpreted as causing a departure from the scope of the disclosure.

The various illustrative logical blocks and modules described in connection with the examples disclosed herein can be implemented or performed by a machine, such as a general purpose processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A general-purpose processor can be a microprocessor, but in the alternative, the processor can be a controller, microcontroller, or state machine, combinations of the same, or the like. A processor can also be implemented as a combination of computing devices, e.g., a combination of a DSP and a microprocessor, a plurality of microprocessors, one or more microprocessors in conjunction with a DSP core, or any other such configuration.

The blocks, operations, or steps of a method, process, or algorithm described in connection with the examples disclosed herein can be embodied directly in hardware, in a software module executed by a processor, or in a combination of the two. A software module can reside in RAM memory, flash memory, ROM memory, EPROM memory, EEPROM memory, registers, hard disk, a removable disk, an optical disc (e.g., CD-ROM or DVD), or any other form of volatile or non-volatile computer-readable storage medium known in the art. A storage medium can be coupled to the processor such that the processor can read information from, and write information to, the storage medium. In the alternative, the storage medium can be integral to the processor. The processor and the storage medium can reside in an ASIC. The ASIC can reside in a user terminal. In the alternative, the processor and the storage medium can reside as discrete components in a user terminal.

Conditional language used herein, such as, among others, “can,” “might,” “may,” “e.g.,” and the like, unless specifically stated otherwise, or otherwise understood within the context as used, is generally intended to convey that some examples include, while other examples do not include, certain features, elements, and/or states. Thus, such conditional language is not generally intended to imply that features, elements, blocks, and/or states are in any way required for one or more examples or that one or more examples necessarily include logic for deciding, with or without author input or prompting, whether these features, elements and/or states are included or are to be performed in any particular example.

The methods disclosed herein may include certain actions taken by a practitioner; however, the methods can also include any third-party instruction of those actions, either expressly or by implication. For example, actions such as “positioning an electrode” include “instructing positioning of an electrode.”

The ranges disclosed herein also encompass any and all overlap, sub-ranges, and combinations thereof. Language such as “up to,” “at least,” “greater than,” “less than,” “between,” and the like includes the number recited. Numbers preceded by a term such as “about” or “approximately” include the recited numbers and should be interpreted based on the circumstances (e.g., as accurate as reasonably possible under the circumstances, for example +5%, +10%, ±15%, etc.). For example, “about 1 V” includes “1 V.” Phrases preceded by a term such as “substantially” include the recited phrase and should be interpreted based on the circumstances (e.g., as much as reasonably possible under the circumstances). For example, “substantially perpendicular” includes “perpendicular.” Unless stated otherwise, all measurements are at standard conditions including temperature and pressure. The phrase “at least one of” is intended to require at least one item from the subsequent listing, not one type of each item from each item in the subsequent listing. For example, “at least one of A, B, and C” can include A, B, C, A and B, A and C, B and C, or A, B, and C.

What is claimed is:

1. A method for optimizing neurostimulation therapy, the method comprising:
measuring a cardiovascular parameter at a first point in time;
applying stimulation at a vector to a nerve proximal to a pulmonary artery;
measuring the cardiovascular parameter at a second point in time;
calculating a change in the cardiovascular parameter; and
determining therapy efficacy of the stimulation at the vector based on the change in the cardiovascular parameter.

2. The method of claim 1, further comprising:
measuring the cardiovascular parameter at a third point in time;
applying stimulation at a second vector to the nerve proximal to the pulmonary artery;
measuring the cardiovascular parameter at a fourth point in time;
calculating a change in the cardiovascular parameter; and
determining therapy efficacy of the stimulation at the second vector based on the change in the cardiovascular parameter.

3. The method of claim 2, further comprising applying the stimulation at the vector with higher therapy efficacy.

4. The method of claim 1, wherein the cardiovascular parameter comprises contractility, preload, and/or chronotropy.

5. The method of claim 1, wherein the vector comprises at least one of electrode position, electrode configuration, or electrode combination.

6. A method for optimizing neurostimulation therapy, the method comprising:
measuring a plurality of cardiovascular parameters at a first point in time, the plurality of cardiovascular parameters comprising at least two of contractility, preload, and chronotropy;
applying stimulation to a nerve at a vector;
measuring the cardiovascular parameters at a second point in time;
calculating changes in the plurality of cardiovascular parameters; and
determining therapy efficacy based on the changes in the plurality of cardiovascular parameters.

7. The method of claim 6, wherein the cardiovascular parameters further comprise lusitropy and systolic blood pressure.

8. The method of claim 6, wherein measuring contractility comprises measuring at least two dimensions of contractility.

9. A method for optimizing neurostimulation therapy, the method comprising:
applying a first stimulation to a nerve proximal to a pulmonary artery;
measuring a cardiovascular parameter at a first point in time;
applying a second stimulation to the nerve in the pulmonary artery with a reduced intensity;
measuring the cardiovascular parameter at a second point in time;
calculating a change in the cardiovascular parameter; and
determining a therapeutic response of the second stimulation based on the change in the cardiovascular parameter.

10. The method of claim 9, further comprising:
upon detecting the therapeutic response above a threshold, applying a third stimulation to the nerve in the pulmonary artery with a further reduced intensity.

11. The method of claim 9, further comprising:
upon detecting the therapeutic response below a threshold, applying a third stimulation with a less reduced intensity.

12. The method of claim 9, further comprising:
upon detecting the therapeutic response below a threshold, stopping applying stimulation.

13. The method of claim 9, wherein the nerve is a nerve of a cardiac plexus.

14. The method of claim 9, wherein the first stimulation comprises pulses having a frequency between 2 Hz and 40 Hz and having a pulse width of 100 μs to 4 ms.

15. The method of claim 1, wherein the nerve is a nerve of a cardiac plexus.

16. The method of claim 1, wherein the stimulation comprises pulses having a frequency between 2 Hz and 40 Hz and having a pulse width of 100 μs to 4 ms and having an intensity of 0.1 mA to 20 mA.

17. The method of claim 6, wherein the nerve is a nerve of a cardiac plexus.

18. The method of claim 6, wherein the stimulation comprises pulses having a frequency between 2 Hz and 40 Hz and having a pulse width of 100 μs to 4 ms and having an intensity of 0.1 mA to 20 mA.
